(19) United States
(12) Patent Application Publication
(10) Pub. No.: US 2005/0137215 A1
(43) Pub. Date: Jun. 23, 2005

(54) [C]-fused bicyclic proline derivatives and their use for treating arthritic conditions

(76) Inventors: Peter R. Guzzo, Niskayuna, NY (US); James M. Hamby, Ann Arbor, MI (US); Matthew R. Johnson, Guilderland, NY (US); Van-Duc Le, Selkirk, NY (US); John E. Mangette, Clifton Park, NY (US); Rajesh A. Shenoy, Selkirk, NY (US); Michael A. Stier, Ypsilanti, MI (US)

Correspondence Address: WARNER-LAMBERT COMPANY, 2800 PLYMOUTH RD, ANN ARBOR, MI, 48105, US

(21) Appl. No.: 10/822,615

(22) Filed: Apr. 12, 2004

Related U.S. Application Data
(60) Provisional application No. 60/463,113, filed on Apr. 15, 2003.

(51) Int. Cl. A61K 31/519; A61K 31/433; A61K 31/4245; C07D 417/02; C07D 413/02

(52) U.S. Cl. 514/265.1; 514/361; 514/362; 514/381; 514/364

(57) ABSTRACT

This invention relates to a compound which is [c]-fused bicyclic proline derivative, or a pharmaceutically acceptable salt thereof; a pharmaceutical composition comprising the compound or the salt thereof, and methods of treating diseases, including, but not limited to, methods of preventing or inhibiting joint cartilage damage and preventing or treating diseases characterized by joint cartilage damage, joint inflammation, or joint pain. The [c]-fused bicyclic proline derivatives are compounds of Formula I as described above. Diseases characterized by joint cartilage damage or joint pain include, for example, osteoarthritis and rheumatoid arthritis. Rheumatoid arthritis is also characterized by joint inflammation. This invention also relates to methods of synthesizing and preparing the [c]-fused bicyclic proline derivatives, or a pharmaceutically acceptable salt thereof.

### CROSS-REFERENCE TO RELATED APPLICATIONS

This application claims benefit of priority from U.S. Provisional Patent Application No. 60/463,113 filed Apr. 15, 2003.

### FIELD OF THE INVENTION

This invention relates to a compound which is a [c]-fused bicyclic proline derivative, or a pharmaceutically acceptable salt thereof; a pharmaceutical composition comprising the compound or the salt thereof, and methods of treating diseases, including, but not limited to, methods of preventing or inhibiting joint cartilage damage and preventing or treating diseases characterized by joint cartilage damage, joint inflammation, or joint pain. Diseases characterized by joint cartilage damage or joint pain include, for example, osteoarthritis and rheumatoid arthritis. Rheumatoid arthritis is also characterized by joint inflammation. This invention also relates to methods of synthesizing and preparing the [c]-fused bicyclic proline derivatives, or a pharmaceutically acceptable salt thereof.

### BACKGROUND OF THE INVENTION

Millions of persons around the world have damage to cartilage leading to degenerative joint disease or osteoarthritis (“OA”). Osteoarthritis is primarily a disorder of cartilage and subchondral bone, although other tissues in and around affected joints are involved. OA is a result of a complex system of interrelated mechanical, biochemical, and molecular mechanisms, and is characterized by joint cartilage damage. The typical OA patient has joint cartilage damage that will eventually lead to joint pain, joint stiffness, joint deformities, and diminishment or loss of joint function. Some OA patients eventually can experience joint inflammation.

No drug has yet been shown to reproducibly alter the natural course of OA or any other disease characterized by joint cartilage damage (see Chapter 18: The Pharmacologic Treatment of Osteoarthritis by Simon, L. S. and Strand, V., in Osteoarthritis, 3^{rd} ed., Moskowitz, R. W. et al. eds., 2001, 1992, 1984, W. B. Saunders Co., New York, p. 371). Further, a few case reports have inferentially suggested that the chronic use of some NSAIDs accelerates joint cartilage damage in OA patients, and some investigators believe the data to be compelling enough to preclude the use of NSAIDs in standard therapy for OA (Chapter 18: The Pharmacologic Treatment of Osteoarthritis by Simon, L. S. and Strand, V., supra, p. 383). The need thus continues for a disease modifying, pharmacologic treatment for diseases characterized by joint cartilage damage.

Applicants have now discovered that novel compounds which are [c]-fused bicyclic proline derivatives, or a pharmaceutically acceptable salt thereof, provide a pharmacologic method for preventing and inhibiting joint cartilage damage, alleviating joint pain, and preventing and treating osteoarthritis, rheumatoid arthritis, and, for that matter, any other disease characterized by joint cartilage damage. Certain of the [c]-fused bicyclic proline derivatives have an additional advantage of not displacing gabapentin from an alpha-2-delta receptor. All that is required to practice the prevention and treatment methods of the instant invention is to administer to a subject in need of treatment of, or at risk for developing, joint cartilage damage, joint pain, osteoarthritis, or any other disease characterized by joint cartilage damage, a therapeutically effective and nontoxic amount of a [c]-fused bicyclic proline derivative, or a pharmaceutically acceptable salt thereof, for the particular condition being prevented or treated.

### SUMMARY OF THE INVENTION

The instant invention provides a compound that is a [c]-fused bicyclic proline derivative, or a pharmaceutically acceptable salt thereof; a pharmaceutical composition comprising the compound or the salt thereof, and methods of preventing or inhibiting joint cartilage damage and preventing or treating diseases characterized by joint cartilage damage, joint inflammation, or joint pain. This invention further relates to methods of synthesizing and preparing the [c]-fused bicyclic proline derivatives, or a pharmaceutically acceptable salt thereof.

More particularly, embodiments of the instant invention include, but are not limited to:

1. A compound of Formula I 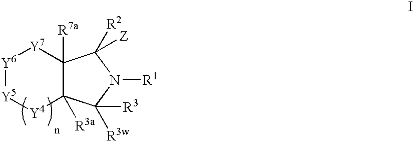
or a pharmaceutically acceptable salt thereof,
wherein:
Z is selected from COOH, C(O)N(H)R⁹, and Z¹;
Z¹ is selected from: 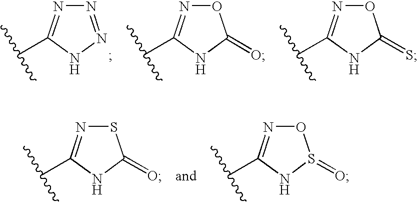
Each Y⁴, Y⁵ Y⁶, and Y⁷ is C(R¹⁰)R^{10w}; or
One of Y⁴, Y⁵, Y⁶, and Y⁷ is selected from O, S, S(O), S(O)₂, and NR⁵, and the other three of Y⁴, Y⁵, Y⁶, and Y⁷ are each C(R¹⁰)R^{10w}; or
Two adjacent Y⁴, Y⁵, Y⁶, and Y⁷ are independently selected from O, S, S(O), S(O)₂, and NR⁵, and the other two of Y⁴, Y⁵, Y⁶, and Y⁷ are each C(R¹⁰)R^{10w};
Each R², R³, R^{3w}, R^{3a}, R^{7a}, R¹⁰, and R^{10w} is independently selected from: H, HO, H₂N, H₂NS(O)₂-(G)ₘ, HS, Halo, CN, CF₃, FC(H)₂O, F₂C(H)O, CF₃O, and
   a group, which may be unsubstituted or substituted, independently selected from:
   C₁-C₆ alkyl-(G)ₘ-, C₂-C₆ alkenyl-(G)ₘ-, C₂-C₆ alkynyl-(G)ₘ-, 2- to 6-membered heteroalkyl-(G)ₘ-, 2- to 6-membered heteroalkenyl-(G)ₘ-, C₃-C₇ cycloalkyl-(G)ₘ-, C₃-C₇ cycloalkenyl-(G)ₘ-, C₇-C₁₀ bicycloalkyl-(G)ₘ-, 3- to 7-membered heterocycloalkyl-(G)ₘ-, 7- to 10-membered heterobicycloalkyl-(G)ₘ-, Phenyl-(G)ₘ-, Naphthyl-(G)ₘ-, 5- and 6-membered heteroaryl-(G)ₘ-, 8- to 10-membered heterobiaryl-(G)ₘ-, and any of the above R², R³, R^{3w}, R^{3a}, R^{7a}, R¹⁰, and R^{10w} groups each independently substituted on carbon or nitrogen atoms with from 1 to 6 substituents R^{X};
wherein R³ and R^{3w}, and any geminal pair of R¹⁰ and R^{10w}, and any two R^{X} substituents geminally substituted on a carbon atom in substituted R², R³, R^{3w}, R^{3a}, R^{7a}, R¹⁰, and R^{10w} groups further may independently be taken together with a carbon atom to which they are both bonded to form the group C(═O);
R¹ is HO or a group that may be unsubstituted or substituted, independently selected from:
   C₁-C₆ alkyl-(T)ₘ-, C₂-C₆ alkenyl-(T)ₘ-, C₂-C₆ alkynyl-(T)ₘ-, 2- to 6-membered heteroalkyl-(T)ₘ-, 2- to 6-membered heteroalkenyl-(T)ₘ-, C₃-C₇ cycloalkyl-(T)ₘ-, C₃-C₇ cycloalkenyl-(T)ₘ-, C₇-C₁₀ bicycloalkyl-(T)ₘ-, 3- to 7-membered heterocycloalkyl-(T)ₘ-, 7- to 10-membered heterobicycloalkyl-(T)ₘ-, Phenyl-(T)ₘ-, Naphthyl-(T)ₘ-, 5- and 6-membered heteroaryl-(T)ₘ-, 8- to 10-membered heterobiaryl-(T)ₘ-, and any of the above R¹ groups independently substituted on a carbon or nitrogen atom, with from 1 to 6 substituents R^{X};
R¹ may further be H when: (i) at least one of R², R³, R^{3w}, R^{3a}, R^{7a}, R¹⁰, and R^{10w} is not H, or (ii) Z is C(O)N(H)R⁹ wherein R⁹ is as defined above wherein m is 1 and L is S(O)₂, or (iv) Z is Z¹;
Each R⁵ and R⁹ is independently H, HO, or a group, which may be unsubstituted or substituted, independently selected from:
   C₁-C₆ alkyl-(L)ₘ-, C₂-C₆ alkenyl-(L)ₘ-, C₂-C₆ alkynyl-(L)ₘ-, 2- to 6-membered heteroalkyl-(L)ₘ-, 2- to 6-membered heteroalkenyl-(L)ₘ-, C₃-C₇ cycloalkyl-(L)ₘ-, C₃-C₇ cycloalkenyl-(L)ₘ-, C₇-C₁₀ bicycloalkyl-(L)ₘ-, 3- to 7-membered heterocycloalkyl-(L)ₘ-, 7- to 10-membered heterobicycloalkyl-(L)ₘ-, Phenyl-(L)ₘ-, Naphthyl-(L)ₘ-, 5- and 6-membered heteroaryl-(L)ₘ-, 8- to 10-membered heterobiaryl-(L)ₘ-, and any of the above R⁵ and R⁹ groups independently substituted, on carbon or nitrogen atoms, with from 1 to 6 substituents R^{X};
wherein any 2 groups each selected from R⁵, R¹⁰, and R^{10w} that are bonded to contiguous carbon or nitrogen atoms in Formula I may be taken together with the contiguous atoms in Formula I to which they are bonded to form C═C or C═N;
wherein any 2 groups selected from R¹, R², R³,R^{3w}, R^{3a},R⁵, R^{7a}, R¹⁰, and R^{10w} that are bonded to contiguous carbon or nitrogen atoms in Formula I may be taken together to form (i) a diradical selected from CH₂ and CH₂CH₂CH₂, (ii) a 3-membered diradical selected from: 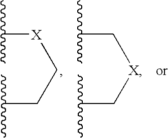
   (iii) a 4-membered diradical selected from: 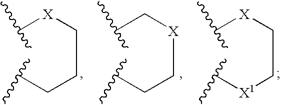
      wherein any two groups R³ and R^{3w}, and R¹⁰ and R^{10w}, that are geminally bonded to a single carbon atom in Formula I may be taken together to form a 4-membered diradical as defined above or a 5-membered diradical selected from: 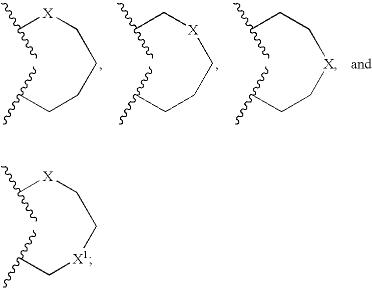
      wherein any 2 groups selected from R¹, R², R³, R^{3w}, R^{3a}, R⁵, R^{7a}, R¹⁰, and R^{10w} that are bonded to noncontiguous carbon or nitrogen atoms in Formula I may be taken together to form (i) a CH₂CH₂ diradical or (ii)-O— diradical;
X is O, S, S(O), S(O)₂, or N—R;
X¹ is O or N—R;
Each G is independently selected from C(═O), S(O), S(O)₂, OC(O), N(R⁴)C(O), (C₁-C₈ alkylenyl)ₘ, (2- to 8-membered heteroalkylenyl)ₘ, and (C₁-C₈ alkylenyl)ₘ and (2- to 8-membered heteroalkylenyl)ₘ independently substituted on carbon or nitrogen atoms with from 1 to 4 substituents R^{X};
Each T is independently selected from S(O), S(O)₂, N(R⁴)C(O), (C₁-C₈ alkylenyl)ₘ, (2- to 8-membered heteroalkylenyl)ₘ, and (C₁-C₈ alkylenyl)ₘ and (2- to 8-membered heteroalkylenyl)ₘ independently substituted on carbon or nitrogen atoms with from 1 to 4 substituents R^{X};
Each L is independently selected from O, N(R⁴), S(O), S(O)₂, C(═O), C(O)O, OC(O), C(O)N(R⁴), N(R⁴)C(O), OC(O)N(R⁴), N(R⁴)C(O)O, N(R⁴)C(O)N(R^{4w}), (C₁-C₈ alkylenyl)ₘ, (2- to 8-membered heteroalkylenyl)ₘ, and (C₁-C₈ alkylenyl)ₘ and (2- to 8-membered heteroalkylenyl)ₘ independently substituted on carbon or nitrogen atoms with from 1 to 4 substituents R^{X};
Each R, R⁴, and R^{4w} is independently H or C₁-C₆ alkyl, which C₁-C₆ alkyl may be unsubstituted or substituted with from 1 to 3 substituents R^{X};
Each R^{X} is independently selected from: HO, H₂N, H₂NS(O)₂, CN, CF₃, FCH₂O, F₂C(H)O, CF₃O, O₂N, C₁-C₆ alkyl-(Q)ₘ-, 2- to 6-membered heteroalkyl-(Q)ₘ-, C₃-C₇ cycloalkyl-(Q)ₘ-, 3- to 7-membered heterocycloalkyl-(Q)ₘ, Phenyl-(Q)ₘ, and 5-membered heteroaryl-(Q)ₘ,
   wherein phenyl and 5-membered heteroaryl-(Q)ₘ each is unsubstituted or independently substituted with from 1 to 3 substituents selected from halo, HO, HOC(O), CH₃OC(O), CH₃C(O), H₂N, CF₃, CN, and C₁-C₆ alkyl;
wherein each R^{X} substituent on a carbon atom may further be independently selected from: HS, (C₁-C₆ alkyl)-S, halo, and HO₂C; and
Each Q independently is O, N(R⁶), S(O), S(O)₂, C(═O), C(O)O, OC(O), C(O)N(R⁶), N(R⁶)C(O), OC(O)N(R⁶), N(R⁶)C(O)O, or N(R⁶)C(O)N(R^{6w});
Each R⁶ and R^{6w} independently is H or unsubstituted C₁-C₆ alkyl;
Each m independently is an integer of 0 or 1; and
Each n independently is an integer of from 0 to 2.

2. A compound of Formula II 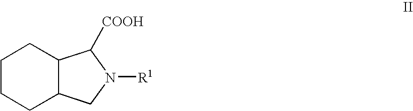
or a pharmaceutically acceptable salt thereof,
wherein R¹ is as defined above for Embodiment 1 wherein R¹ is not H.

3. A compound of Formula III 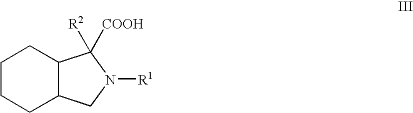
or a pharmaceutically acceptable salt thereof,
wherein R¹ and R² are as defined above for Embodiment 1 wherein R¹ and R² are not both H.

4. A compound of Formula IV 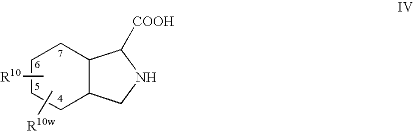
or a pharmaceutically acceptable salt thereof,
wherein each R¹⁰ and R^{10w} is independently bonded to any one of the 4-position to the 7-position in Formula IV, and each is independently as defined above for Embodiment 1 wherein R¹⁰ is not H.

5. A compound of Formula V 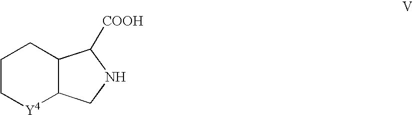
or a pharmaceutically acceptable salt thereof,
wherein Y⁴ is selected from O, S, S(O), S(O)₂, and NR⁵, wherein R⁵ is as defined above for Embodiment 1.

6. A compound of Formula VI 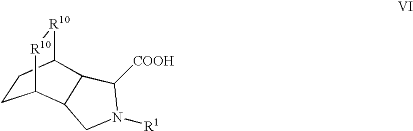
or a pharmaceutically acceptable salt thereof,
wherein each R¹⁰ is bonded to noncontiguous carbon atoms and are taken together to form (i) a CH₂ diradical, (ii) a CH₂CH₂ diradical, or (iii) —O— diradical, and R¹ is independently as defined above for Embodiment 1.

7. A compound of Formula VII 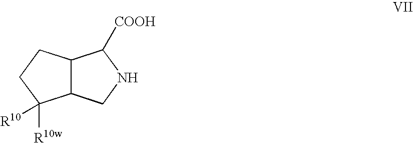
or a pharmaceutically acceptable salt thereof,
wherein R¹⁰ and R^{10w} are independently as defined above for Embodiment 1.

8. A compound of Formula VIII 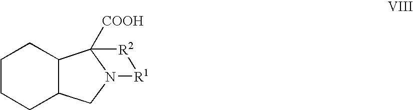
or a pharmaceutically acceptable salt thereof,
wherein R¹ and R² are bonded to contiguous carbon and nitrogen atoms and are taken together to form a diradical selected from CH₂ and CH₂CH₂CH₂.

9. A compound of Formula IX 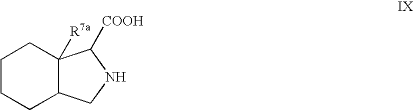
or a pharmaceutically acceptable salt thereof,
wherein R^{7a} is as defined for Embodiment 1.

10. A compound of Formula X 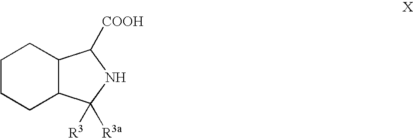
or a pharmaceutically acceptable salt thereof,
wherein R³ and R^{3a} are as defined for Embodiment 1.

11. A compound of Formula XI 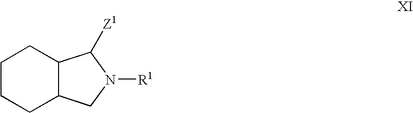
or a pharmaceutically acceptable salt thereof,
wherein R¹ and Z¹ are as defined for Embodiment 1.

12. The compound according to any one of Embodiments 1, 2, 3, 6, and 11, wherein R is not H.

13. The compound according to any one of Embodiments 1, 2, 3, 6, 11, and 12, wherein R¹ is unsubstituted or substituted C₁-C₆ alkyl-(L)ₘ, CH₃, CH₂CH₃, or CH₂CH₂CH₃.

14. The compound according to any one of Embodiments 1, 2, 3, 6, 11, 12, and 13, wherein R¹ is CH₃.

15. The compound according to any one of Embodiments 1, 4, and 7 wherein R¹ is H and at least one of R¹⁰ and R^{10w} is not H.

16. The compound according to any one of Embodiments 1 and 3, wherein R² is CH₃, CH₂CH₃ or CH₂CH₂CH₃.

17. The compound according to Embodiment 1, wherein R² is H.

18. The compound according to Embodiment 1, wherein at least one of R¹, R⁵, R⁹ is selected from unsubstituted and substituted C₁-C₆ alkyl-(L)ₘ, C₂-C₆ alkenyl-(L)., and C₂-C₆ alkynyl-(L)ₘ.

19. The compound according to Embodiment 1, wherein at least one of R¹, R⁵, and R⁹ is selected from unsubstituted and substituted 2- to 6-membered heteroalkyl-(L)ₘ and 2- to 6-membered heteroalkenyl-(L)ₘ.

20. The compound according to Embodiment 1, wherein at least one of R¹, R⁵, and R⁹ is selected from unsubstituted and substituted C₃-C₇ cycloalkyl-(L)ₘ, C₃-C₇ cycloalkenyl-(L)ₘ, and C₇-C₁₀ bicycloalkyl-(L)ₘ.

21. The compound according to Embodiment 1, wherein at least one of R¹, R⁵, and R⁹ is selected from unsubstituted and substituted 3- to 7-membered heterocycloalkyl-(L)ₘ and 7- to 10-membered heterobicycloalkyl-(L)ₘ.

22. The compound according to Embodiment 1, wherein at least one of R¹, R⁵, and R⁹ is selected from unsubstituted and substituted phenyl-(L)ₘ and naphthyl-(L)ₘ.

23. The compound according to Embodiment 1, wherein at least one of R¹, R⁵, and R⁹ is selected from unsubstituted and substituted 5- and 6-membered heteroaryl-(L)ₘ and 8- to 10-membered heterobiaryl-(L)ₘ.

24. The compound according to Embodiment 1, wherein at least one of R¹, R⁵, and R⁹ is H or OH.

25. The compound according to any one of Embodiments 1, 3, 4, 7, 9, 10, 16, and 17, wherein at least one of R², R³,R^{3w}, R^{3a}, R^{7a}, R¹⁰, and R^{10w} is selected from unsubstituted and substituted C₁-C₆ alkyl-(L)ₘ, C₂-C₆ alkenyl-(L)ₘ, and C₂-C₆ alkynyl-(L)ₘ.

26. The compound according to any one of Embodiments 1, 3, 4, 7, 9, 10, 16, and 17, wherein at least one of R², R³, R^{3w}, R^{3a}, R^{7a}, R¹⁰, and R^{10w} is selected from unsubstituted and substituted 2- to 6-membered heteroalkyl-(L)ₘ and 2- to 6-membered heteroalkenyl-(L)ₘ.

27. The compound according to any one of Embodiments 1, 3, 4, 7, 9, 10, 16, and 17, wherein at least one of R², R³,R^{3w}, R^{3a}, R^{7a}, R¹⁰, and R^{10w} is selected from unsubstituted and substituted C₃-C₇ cycloalkyl-(L)ₘ, C₃-C₇ cycloalkenyl-(L)ₘ, and C₇-C₁₀ bicycloalkyl-(L)ₘ.

28. The compound according to any one of Embodiments 1, 3, 4, 7, 9, 10, 16, and 17, wherein at least one of R², R³, R^{3w}, R^{3a}, R^{7a}, R¹⁰, and R^{10w} is selected from unsubstituted and substituted 3- to 7-membered heterocycloalkyl-(L)ₘ and 7- to 10-membered heterobicycloalkyl-(L)ₘ.

29. The compound according to any one of Embodiments 1, 3, 4, 7, 9, 10, 16, and 17, wherein at least one of R², R³, R^{3w}, R^{3a}, R^{7a}, R¹⁰, and R^{10w} is selected from unsubstituted and substituted phenyl-(L)ₘ and naphthyl-(L)ₘ.

30. The compound according to any one of Embodiments 1, 3, 4, 7, 9, 10, 16, and 17, wherein at least one of R², R³, R^{3w}, R^{3a}, R^{7a}, R¹⁰, and R^{10w} is selected from unsubstituted or substituted 5- and 6-membered heteroaryl-(L)ₘ and 8- to 10-membered heterobiaryl-(L)ₘ.

31. The compound according to any one of Embodiments 1, 3, 4, 7, 9, 10, 16, and 17, wherein at least one of R², R³, R^{3w}, R^{3a}, R^{7a}, R¹⁰, and R^{10w} is selected from OH, FCH₂O, F₂CHO, and CF₃O.

32. The compound according to any one of Embodiments 1, 3, 4, 7, 9, 10, 16, and 17, wherein at least one of R², R³, R^{3w}, R^{3a}, R^{7a}, R¹⁰, and R^{10w} is selected from CN and CF₃.

33. The compound according to any one of Embodiments 1, 3, 4, 7, 9, 10, 16, and 17, wherein at least one of R², R³, R^{3w}, R^{3a}, R^{7a}, R¹⁰, and R^{10w} is halo.

34. The compound according to any one of Embodiments 1, 3, 4, 7, 9, 10, 16, and 17, wherein at least one of R², R³, R^{3w}, R^{3a}, R^{7a}, R¹⁰, and R^{10w} is H₂N or H₂NS(O)₂-(G)ₘ.

35. The compound according to any one of Embodiments 1, 3, 4, 7, 9, 10, and 17, wherein each of R², R³, R^{3w}, R^{3a}, R^{7a}, R¹⁰, and R^{10w} is H.

36. The compound according to any one of Embodiments 1, 3, 4, 7, 9, 10, 16, and 17, wherein one of R³ and R^{3w} and any geminal pair of R¹⁰ and R^{10w} is independently taken together with a carbon atom to which they are both bonded to form the group C(═O).

37. The compound according to Embodiment 1, wherein each of Y⁴, Y⁵, Y⁶, and Y⁷ is C(R¹⁰)R^{10w}.

38. The compound according to Embodiment 1, wherein one of Y⁵, Y⁶, and Y⁷ is selected from O, S, S(O), S(O)₂, and NR⁵, and Y⁴ and the others of Y⁵, Y⁶, and Y⁷ are each C(R¹⁰)R^{10w}.

39. The compound according to Embodiment 1, wherein two nonadjacent Y⁴, Y⁵, Y⁶, and Y⁷ are independently selected from O, S, S(O), S(O)₂, and NR⁵, and the other two of Y⁴, Y⁵, Y⁶ and Y⁷ are each C(R¹⁰)R^{10w}.

40. The compound according to any one of Embodiments 1 to 39, wherein R, R⁴, R^{4w}, R⁶, and R^{6w} are each independently H or CH₃.

41. The compound according to any one of Embodiments 1, 3 to 10, 12, 13, and 15 to 40, wherein at least one of G, L, and Q independently is selected from O, N(R⁴), S(O), and S(O)₂.

42. The compound according to any one of Embodiments 1, 3 to 10, 12, 13, and 15 to 40, wherein at least one of G, L, and Q independently is selected from C(═O), C(O)O, and OC(O).

43. The compound according to any one of Embodiments 1, 3 to 10, 12, 13, and 15 to 40, wherein at least one of G and L independently is selected from C(O)N(R⁴), N(R⁴)C(O), OC(O)N(R⁴), N(R⁴)C(O)O, and N(R⁴)C(O)N(R^{4w}).

44. The compound according to any one of Embodiments 1, 3 to 10, 12, 13, and 15 to 40, wherein Q independently is selected from C(O)N(R⁶), N(R⁶)C(O), OC(O)N(R⁶), N(R⁶)C(O)O, and N(R⁶)C(O)N(R^{6w}).

45. The compound according to any one of Embodiments 1 and 12 to 44, wherein Z is COOH.

46. The compound according to any one of Embodiments 1 and 12 to 44, wherein Z is C(O)N(H)R⁹.

47. The compound according to any one of Embodiments 1 and 12 to 44, wherein Z is not Z¹.

48. The compound according to any one of Embodiments 1 and 12 to 44, wherein Z is

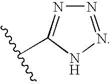

49. The compound according to any one of Embodiments 1 and 12 to 44, wherein Z is

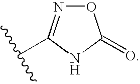

50. The compound according to any one of Embodiments 1 and 12 to 44, wherein Z is

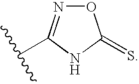

51. The compound according to any one of Embodiments 1 and 12 to 44, wherein Z is

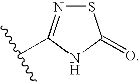

52. The compound according to any one of Embodiments 1 and 12 to 44, wherein Z is

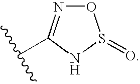

53. The compound according to any one of Embodiments 1 and 12 to 52, wherein n is 0.

54. The compound according to any one of Embodiments 1 and 12 to 52, wherein n is 1.

55. The compound according to any one of Embodiments 1 and 12 to 52, wherein n is 2.

56. The compound according to any one of Embodiments 1, 3 to 10, 12, 13, and 15 to 55, wherein each m is 0.

57. The compound according to any one of Embodiments 1, 3 to 10, 12, 13, and 15 to 55, wherein at least two m groups are 1.

58. A compound selected from:
(1S,3aS,7aS)-2-methyl-1,3,3a,4,5,6,7,7a-octahydroisoindole-1-carboxylic acid;
(1S,3aS,7aR)-2-methyl-1,3,3a,4,5,6,7,7a-octahydroisoindole-1-carboxylic acid;
(1R,3aR,7aS)-2-methyl-1,3,3a,4,5,6,7,7a-octahydroisoindole-1-carboxylic acid;
(1S,3aR,7aS)-2-methyl-1,3,3a,4,5,6,7,7a-octahydroisoindole-1-carboxylic acid;
(1R,3aS,7aR)-2-methyl-1,3,3a,4,5,6,7,7a-octahydroisoindole-1-carboxylic acid;
(1S,3aR,7aR)-2-methyl-1,3,3a,4,5,6,7,7a-octahydroisoindole-1-carboxylic acid;
(1R,3aS,7aS)-2-methyl-1,3,3a,4,5,6,7,7a-octahydroisoindole-1-carboxylic acid; and
(1R,3aR,7aR)-2-methyl-1,3,3a,4,5,6,7,7a-octahydroisoindole-1-carboxylic acid; or
   a pharmaceutically acceptable salt thereof.

59. The compound according to Embodiment 1, or a pharmaceutically acceptable salt thereof, wherein any 2 groups each selected from R⁵, R¹⁰, and R^{10w} that are bonded to contiguous carbon or nitrogen atoms in Formula I may not be taken together with the contiguous atoms in Formula I to which they are bonded to form C═C or C═N.

60. The compound according to Embodiment 1, or a pharmaceutically acceptable salt thereof, wherein any 2 groups selected from R¹, R², R³, R^{3w}, R^{3a}, R⁵, R^{7a}, R¹⁰, and R^{10w} that are bonded to contiguous carbon or nitrogen atoms in Formula I may not be taken together to form (i) a diradical selected from CH₂ and CH₂CH₂CH₂, (ii) a 3-membered diradical selected from: 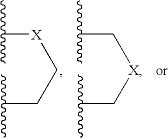
(iii) a 4-membered diradical selected from: 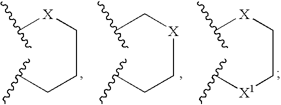
wherein any two groups R³ and R^{3w 3}, and R¹⁰ and R^{10w}, that are geminally bonded to a single carbon atom in Formula I may be taken together to form a 4-membered diradical as defined above or a 5-membered diradical selected from: 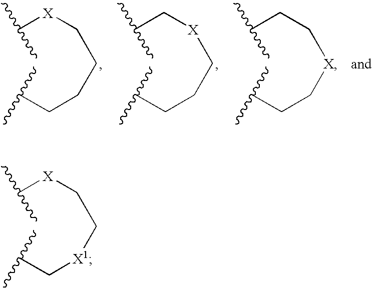
   wherein X and X¹ are as defined above in Embodiment 1.

61. The compound according to Embodiment 1, or a pharmaceutically acceptable salt thereof, wherein any 2 groups selected from R¹, R², R³, R^{3w}, R^{3a}, R⁵, R^{7a}, R¹⁰, and R^{10w} that are bonded to noncontiguous carbon or nitrogen atoms in Formula I may not be taken together to form (i) a CH₂CH₂ diradical or (ii) —O— diradical.

62. A pharmaceutical composition, comprising a compound according to Embodiment 1, or a pharmaceutically acceptable salt thereof, and a pharmaceutically acceptable carrier, diluent, or excipient.

63. The pharmaceutical composition according to Embodiment 62, wherein the compound is according to any one of Embodiments 2 to 61.

64. The pharmaceutical composition according to Embodiment 62, wherein the compound is according to any one of the below Compound Examples.

65. The pharmaceutical composition according to any one of Embodiments 62 to 64, wherein the compound is in solid dosage form.

66. The pharmaceutical composition according to any one of Embodiments 62 to 65, wherein the compound is in solid dosage form in an amount of from 1 milligram to 1000 milligrams, 10 to 750 milligrams, 20 to 500 milligrams, 50 to 400 milligrams, or 100 to 300 milligrams.

67. A method of inhibiting joint cartilage damage in a mammal in need thereof, comprising administering to the mammal a joint cartilage damage inhibiting effective amount of a compound according to Embodiment 1, or a pharmaceutically acceptable salt thereof.

68. The method according to Embodiment 67, wherein the joint cartilage damage is not accompanied by inflammation or pain in the joint.

69. The method according to Embodiment 67, wherein the joint cartilage damage is not accompanied by inflammation in the joint.

70. The method according to Embodiment 67, wherein the joint cartilage damage is not accompanied by pain in the joint.

71. A method of treating osteoarthritis in a mammal in need thereof, comprising administering to the mammal an osteoarthritis treating effective amount of a compound according to Embodiment 1, or a pharmaceutically acceptable salt thereof.

72. A method of treating rheumatoid arthritis in a mammal in need thereof, comprising administering to the mammal an rheumatoid arthritis treating effective amount of a compound according to Embodiment 1, or a pharmaceutically acceptable salt thereof.

73. A method of treating joint inflammation in a mammal in need thereof, comprising administering to the mammal an antiinflammatory effective amount of a compound according to Embodiment 1, or a pharmaceutically acceptable salt thereof.

74. A method of alleviating joint pain in a mammal in need thereof, comprising administering to the mammal a joint pain alleviating effective amount of a compound according to Embodiment 1, or a pharmaceutically acceptable salt thereof.

75. The method according to Embodiment 74, wherein the joint pain is an inflammatory joint pain.

76. The method according to Embodiment 74, wherein the joint pain is an osteoarthritic pain.

77. The method according to Embodiment 74, wherein the joint pain is a rheumatoid arthritic pain.

78. The method according to Embodiment 74, wherein the joint pain is accompanied by cartilage damage to the joint.

79. The method according to Embodiment 74, wherein the joint pain is not accompanied by cartilage damage to the joint.

80. A method of alleviating acute pain in a mammal in need thereof, comprising administering to the mammal an acute pain alleviating effective amount of a compound according to Embodiment 1, or a pharmaceutically acceptable salt thereof.

81. A method of alleviating chronic pain in a mammal in need thereof, comprising administering to the mammal a chronic pain alleviating effective amount of a compound according to Embodiment 1, or a pharmaceutically acceptable salt thereof.

82. The method according to any one of Embodiments 67 to 81, wherein the compound is according to any one of Embodiments 2 to 61.

83. The method according to any one of Embodiments 67 to 81, wherein the compound is according to any one of the below Compound Examples.

The present invention also provides a method of treating a disease in a mammal suffering therefrom, comprising administering to the mammal a therapeutically effective amount of a compound of any one of Embodiments 1 to 61 or any one of the below Compound Examples, or a pharmaceutically acceptable salt thereof, wherein the disease is selected from an autoimmune disease, a rheumatic disease, and an inflammatory skin disease.

A preferred method of treating a rheumatic disease is a method that treats ankylosing spondylitis, arthritis, avascular necrosis, Behçet's syndrome, end stage lung disease, fibromyalgia, gout, polymyalgia rheumatica, giant cell arteritis, HIV-associated rheumatic syndromes, neurogenic arthropathy, osteoporosis, pseudogout, psoriasis, Reiter's syndrome, scleroderma, Sjörgren's disease, Still's disease, bursitis, tendonitis, ulcerative colitis, vasculitis, or Wegener's granulomatosis.

A preferred method of treating arthritis is a method that treats osteoarthritis, rheumatoid arthritis, psoriatic arthritis, juvenile arthritis, reactive arthritis, Lyme arthritis, or infectious arthritis. A more preferred method of treating arthritis is a method that treats osteoarthritis or rheumatoid arthritis.

A preferred method of treating an inflammatory skin disease is a method that treats psoriasis, eczema, atopic dermatitis, contact dermatitis, discoid lupus, pemphigus vulgaris, bullous pemphigoid, and alopecia areata. A more preferred methods of treating an inflammatory skin disease is a method that treats psoriasis, eczema, or atopic dermatitis.

A preferred method of treating an autoimmune disease is a method that treats an autoimmune disease of the nervous system, blood, gastrointestinal system, endocrine glands, skin, or musculoskeletal system.

A more preferred method of treating an autoimmune disease is a method that treats an autoimmune disease of the nervous system selected from multiple sclerosis, myasthenia gravis, autoimmune neuropathies including Guillian-Barré, and autoimmune uveitis.

Another more preferred method of treating an autoimmune disease is a method that treats an autoimmune disease of the blood selected from temporal arteritis, anti-phospholipid syndrome, vasculitides including Wegener's granulomatosis, and Behçet's disease.

Another more preferred method of treating an autoimmune disease is a method that treats an autoimmune disease of the gastrointestinal system selected from Crohn's disease, ulcerative colitis, primary biliary cirrhosis, and autoimmune hepatitis.

Another more preferred method of treating an autoimmune disease is a method that treats an autoimmune disease of the endocrine glands selected from Type-1 or immune mediated diabetes mellitus, Grave's disease, Hashimoto's thyroiditis, autoimmune oophoritis, autoimmune orchitis, and autoimmune disease or the adrenal gland.

Another more preferred method of treating an autoimmune disease is a method that treats an autoimmune disease of the musculoskeletal system selected from rheumatoid arthritis, systemic lupus erythematosus, scleroderma, polymyositis, dermatomyositis, spondyloarthropathies including ankylosing spondylitis, and Sjörgren's syndrome.

The present invention also provides a method of treating a disease in a mammal suffering therefrom, comprising administering to the mammal a therapeutically effective amount of a compound of any one of Embodiments 1 to 61 or any one of the below Compound Examples, or a pharmaceutically acceptable salt thereof, wherein the compound is a ligand to an alpha-2-delta receptor with an IC₅₀ of less than 1 μM, preferably less than 0.1 μM, determined with pig alpha-2-delta receptor 1 according to Biological Method 5 and wherein the disease is selected from anxiety, fibromyalgia, and sleep disruption due to fibromyalgia.

Another invention embodiment is an ester of Formula (E) 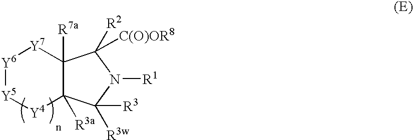
or a pharmaceutically acceptable salt thereof,
wherein R¹, R², R³, R^{3w}, R^{3a}, R^{7a}, Y⁴, Y⁵, Y⁶, Y⁷, and n are as defined above for Formula I and
R⁸ is a group independently selected from: C₁-C₆ alkyl, C₂-C₆ alkenyl, C₂-C₆ alkynyl, 2- to 6-membered heteroalkyl, 2- to 6-membered heteroalkenyl, C₃-C₇ cycloalkyl, C₃-C₇ cycloalkenyl, C₇-C₁₀ bicycloalkyl, 3- to 7-membered heterocycloalkyl, 7- to 10-membered heterobicycloalkyl, phenyl, naphthyl, 5- and 6-membered heteroaryl, 8- to 10-membered heterobiaryl, and any of the above R⁸ groups independently substituted on a carbon or nitrogen atom, with from 1 to 6 substituents R^{X};
wherein any two R^{X} substituents geminally substituted on a carbon atom in a substituted R⁸ group may optionally be taken together with a carbon atom to which they are both bonded to form the group C(═O); and
R¹ may further be H when R⁸ is not unsubstituted C₁-C₆ alkyl or benzyl.

One particular embodiment is the compound of Formula (E) wherein R⁸ is 3-dimethylamino-2,2-dimethyl-propyl, 2-dimethylamino-ethyl, or 2-dimethylamino-2,2-dimethyl-ethyl.

Another embodiment of the present invention is a compound of Formula (E) named (1S,3aS,7aS)-octahydroisoindole-1-carboxylic acid 2-dimethylamino-2,2-dimethyl-ethyl ester.

A compound of Formula (E) is useful as an intermediate in the preparation of a compound of Formula I and as a prodrug of a compound of Formula I. Further, the compound of Formula (E) per se may be useful in any of the pharmaceutical compositions and methods of treating embodiments described above for a compound of Formula I.

Another aspect of this invention is use of a compound according to any one of the embodiments described herein, or a pharmaceutically acceptable salt thereof, in the preparation of a medicament that is useful for treating joint cartilage damage, osteoarthritis, rheumatoid arthritis, or joint inflammation, or alleviating joint pain, in a mammal suffering from joint cartilage damage, osteoarthritis, rheumatoid arthritis, joint inflammation, or joint pain, respectively.

### DETAILED DESCRIPTION OF THE INVENTION

This invention relates to a compound which is a [c]-fused bicyclic proline derivative of Formula I, or a pharmaceutically acceptable salt thereof; a pharmaceutical composition comprising the compound or the salt thereof, and methods of treating diseases, including, but not limited to, preventing or inhibiting joint cartilage damage and preventing or treating diseases characterized by joint cartilage damage, joint inflammation, or joint pain. Diseases characterized by joint cartilage damage or joint pain include, for example, osteoarthritis and rheumatoid arthritis. Rheumatoid arthritis is also characterized by joint inflammation. This invention also relates to methods of synthesizing and preparing the [c]-fused bicyclic proline derivatives, or a pharmaceutically acceptable salt thereof.

### Additional Embodiments

Nonlimiting examples of additional invention embodiments are described below.

Another invention embodiment comprises invention compounds that do not displace (i.e., IC₅₀≧10 micromolar) gabapentin from an alpha-2-delta receptor subtype 1 or 2.

Another invention embodiment comprises invention compounds that weakly displace (i.e., 1 micromolar≦IC₅₀<10 micromolar) gabapentin from an alpha-2-delta receptor subtype 1 or 2.

Another invention embodiment comprises invention compounds that displace (i.e., IC₅₀<1 micromolar) gabapentin from an alpha-2-delta receptor subtype 1 or 2.

Another invention embodiment is a method of treating joint cartilage damage, joint inflammation, joint pain, osteoarthritis, or rheumatoid arthritis in a mammal suffering therefrom, comprising administering to the mammal a therapeutically effective amount of a compound of any one of Embodiments 1 to 61 or any one of the below Compound Examples, or a pharmaceutically acceptable salt thereof, wherein the compound is characterized as having an IC₅₀ of greater than or equal to 1 μM, preferably greater than or equal to 10 μM, determined with pig alpha-2-delta receptor 1 according to Biological Method 5.

Another invention embodiment comprises invention compounds that do not bind (i.e., IC₅₀≧10 millimolar) to a leucine transport system.

Another invention embodiment comprises invention compounds that very weakly bind (i.e., 1 millimolar≦IC₅₀<10 millimolar) to a leucine transport system.

Another invention embodiment comprises invention compounds that weakly bind (i.e., 1 micromolar≦IC₅₀<1 millimolar) to a leucine transport system.

Another invention embodiment comprises invention compounds that bind (i.e., IC₅₀<1 micromolar) to a leucine transport system.

Another invention embodiment is a method of treating joint cartilage damage, joint inflammation, joint pain, osteoarthritis, or rheumatoid arthritis in a mammal suffering therefrom, comprising administering to the mammal a therapeutically effective amount of a compound of any one of Embodiments 1 to 61 or any one of the below Compound Examples, or a pharmaceutically acceptable salt thereof, wherein the compound is characterized as having an IC₅₀ of greater than or equal to 1 μM, preferably greater than or equal to 10 μM, determined with CHO K1 cells according to Biological Method 7.

Another invention embodiment is any one of the above-recited method of uses or any one of the below-recited combinations, wherein the active invention compound is any stereoisomer of octahydroisoindole-1-carboxylic acid, or a pharmaceutically acceptable salt thereof, instead of the compound of Formula I, or a pharmaceutically acceptable salt thereof. Preferred is (1S,3aS,7aS)-octahydroisoindole-1-carboxylic acid, or a pharmaceutically acceptable salt thereof.

This invention also includes combinations comprising a compound of Formula I or a compound of Formula (E) with a second therapeutic agent as described below, pharmaceutical compositions comprising the combinations, and methods of inhibiting joint cartilage damage in a mammal, treating osteoarthritis, rheumatoid arthritis, or joint inflammation in a mammal, or alleviating joint pain in a mammal, comprising administering to the mammal an effective amount of any of the combinations or pharmaceutical compositions containing the compositions.

Many invention compounds are amphoteric, and are thus capable of further forming pharmaceutically acceptable salts, including, but not limited to, acid addition and base addition salts. All pharmaceutically acceptable salt forms of the invention compounds are included within the scope of the present invention.

Pharmaceutically acceptable acid addition salts of an invention compound include salts derived from inorganic acids such as hydrochloric, nitric, phosphoric, sulfuric, hydrobromic, hydroiodic, hydrofluoric, phosphorous, and the like, as well salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, malate, tartrate, methanesulfonate, and the like. Also contemplated are salts of amino acids such as arginate and the like and gluconate, galacturonate (see, for example, Berge S. M. et al., “Pharmaceutical Salts,” J. of Pharma. Sci., 1977; 66:1).

An acid addition salt of an invention compound is prepared by contacting the free base form of the compound with a sufficient amount of a desired acid to produce the salt in a conventional manner. The acid addition salt may be converted back to the free base form of the invention compound by contacting the acid addition salt with a base, and isolating the free base form of the compound in a conventional manner. The free base forms of the invention compounds differ from their respective acid addition salt forms somewhat in certain physical properties such as solubility, dissolution rate, crystal structure, hygroscopicity, and the like, but otherwise the free base forms of the compounds and their respective acid addition salt forms are equivalent for purposes of the present invention.

A pharmaceutically acceptable base addition salt of an invention compound may be prepared by contacting the free acid form of the compound with a sufficient amount of a desired base containing a metal cation such as an alkali or alkaline earth metal cation, or with an amine, especially an organic amine, to produce the salt in the conventional manner. Examples of suitable metal cations include sodium cation (Na⁺), potassium cation (K⁺), magnesium cation (Mg²⁺), calcium cation (Ca²⁺), and the like. Examples of suitable amines are N,N′-dibenzylethylenediamine, chloroprocaine, choline, diethanolamine, dicyclohexylamine, ethylenediamine, N-methylglucamine, and procaine (see, for example, Berge, supra., 1977).

A base addition salt of an invention compound may be converted back to the free acid form of the compound by contacting the base-addition salt with an acid, and isolating the free acid of the invention compound in a conventional manner. The free acid forms of the invention compounds differ from their respective base addition salt forms somewhat in certain physical properties such as solubility, dissolution rate, crystal structure, hygroscopicity, and the like, but otherwise the base addition salts are equivalent to their respective free acid forms for purposes of the present invention.

The invention compounds can exist in unsolvated forms as well as solvated forms, including hydrated forms. In general, the solvated forms, including hydrated forms, are equivalent to unsolvated forms and are included within the scope of the present invention. The present invention includes any unsolvated or solvated form of a compound of Formula I, or a pharmaceutically acceptable salt thereof.

Certain invention compounds can exist as crystalline solids. Each invention compound capable of existing as a crystalline solid may crystallize in one or more polymorphic forms depending on the conditions used for crystallization. All polymorphic forms of crystalline invention compounds are encompassed within the scope of the present invention.

The invention compounds possess chiral centers, and each center may exist in the R or S configuration. The present invention includes any stereoisomer of a compound of Formula I, or a pharmaceutically acceptable salt thereof, including any diastereomeric, enantiomeric, or epimeric form of the invention compounds, as well as mixtures thereof.

Additionally, certain invention compounds may exist as geometric isomers such as the entgegen (E) and zusammen (Z) isomers of 1,2-disubstituted alkenyl groups or cis and trans isomers of disubstituted cyclic groups. An invention compound includes any cis, trans, syn, anti, entgegen (E), or zusammen (Z) isomer of the compound, as well as mixtures thereof.

Certain invention compounds can exist as two or more tautomeric forms. Tautomeric forms of the invention compounds are forms that may interchange by shifting of the position of a hydrogen atom and a bond(s), for example, via enolization/de-enolization, 1,2-hydride, 1,3-hydride, or 1,4-hydride shifts, and the like. Tautomeric forms of an invention compound are isomeric forms of the invention compound that exist in a state of equilibrium, wherein the isomeric forms of the invention compound have the ability to interconvert by isomerization in situ, including in a reaction mixture, in an in vitro biological assay, or in vivo. An invention compound includes any tautomeric form of the compound, as well as mixtures thereof.

Some compounds of the present invention have alkenyl groups, which may exist as entgegen or zusammen conformations, in which case all geometric forms thereof, both entgegen and zusammen, cis and trans, and mixtures thereof, are within the scope of the present invention.

Some compounds of the present invention have cycloalkyl groups, which may be substituted at more than one carbon atom, in which case all geometric forms thereof, both cis and trans, and mixtures thereof, are within the scope of the present invention.

The invention compounds also include isotopically-labelled compounds, which are identical to those recited above, but for the fact that one or more atoms are replaced by an atom having an atomic mass or mass number different from the atomic mass or mass number usually found in nature. Examples of isotopes that can be incorporated into the invention compounds include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F and ³⁶Cl, respectively. The invention compounds and their pharmaceutically acceptable salts which contain the aforementioned isotopes and/or other isotopes of other atoms are within the scope of this invention.

Certain isotopically labelled invention compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution of atoms in invention compounds with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of those described above in this invention can generally be prepared by art recognized procedures, or by carrying out the procedures incorporated by reference below, or procedures disclosed in the Schemes and/or in the Examples and Preparations, if any, below, by substituting a readily available isotopically labelled reagent for a non-isotopically labelled reagent.

It should also be appreciated that, in order to be concise, the instant invention compound of Formula I as defined above includes many embodiments not specifically described herein. These embodiments would be nevertheless readily known to one of ordinary skill in the art, and are embraced herein. These embodiments include, for example, independently within each group defined for Formula I, including the R groups R, R¹, R², R³, R^{3w}, R^{3a}, R⁴, R^{4w}, R⁵,R⁶, R^{6w}, R^{7a}, R⁹, R¹⁰, R^{10w}, and R^{X}, permutations of terms such as, for example, C₁-C₆ alkyl and 5- and 6-membered heteroaryl groups.

For illustration purposes, permutations of C₁-C₆ alkyl groups include embodiments selected from: C₁ alkyl; C₂ alkyl; C₃ alkyl; C₄ alkyl; C₅ alkyl; C₆ alkyl; C₁ and C₂ alkyl; C₃ and C₆ alkyl; C₅ and C₆ alkyl; C₁-C₃ alkyl; C₃-C₅ alkyl; C₂, C₄, and C₆ alkyl; C₂-C₅ alkyl; C₁, C₃, C₅, and C₆ alkyl; C₁-C₅ alkyl; C₁-C₄ and C₆ alkyl; C₁ and C₃-C₆ alkyl; C₂-C₆ alkyl; and the like.

For illustration purposes, permutations of 5- and 6-membered heteroaryl groups include, for illustration, embodiments selected from: 5-membered heteroaryl; 6-membered heteroaryl; isothiazolyl, isoxazolyl, oxadiazolyl, oxazolyl, purinyl, pyrazinyl, pyridazinyl, pyridinyl, pyrimidinyl, pyrazolyl, pyrrolyl, quinazolinyl, quinolinyl, quinoxalinyl, tetrazolyl, thiazolyl, thiadiazolyl, thienyl, triazinyl, and triazolyl; isothiazolyl, isoxazolyl, oxadiazolyl, oxazolyl, purinyl, pyrazinyl, pyridazinyl, pyridinyl, pyrimidinyl, pyrazolyl, quinazolinyl, quinolinyl, quinoxalinyl, tetrazolyl, thiazolyl, thiadiazolyl, thienyl, triazinyl, and triazolyl; oxazolyl, purinyl, pyrazinyl, pyridazinyl, pyridinyl, pyrimidinyl, pyrazolyl, pyrrolyl, quinazolinyl, quinolinyl, quinoxalinyl, tetrazolyl, thiazolyl, thiadiazolyl, thienyl, triazinyl, and triazolyl; isothiazolyl, oxadiazolyl, purinyl, pyrazinyl, pyridazinyl, pyridinyl, pyrazolyl, tetrazolyl, thiazolyl, thiadiazolyl, triazinyl, and triazolyl; isothiazolyl, isoxazolyl, and oxadiazolyl; oxazolyl and purinyl; isoxazolyl and oxadiazolyl; tetrazolyl; thiazolyl; thiadiazolyl; thienyl; and triazolyl.

Accordingly, embodiments of the present invention compounds of Formula I that are not specifically described above find support in instant the specification and may be claimed in the future in this application or any continuations, continuations-in-part, and divisionals thereof.

One of ordinary skill in the art will appreciate that the compounds of the present invention that displace gabapentin from an alpha-2-delta receptor are useful in treating a diverse array of diseases wherein binding to an alpha-2-delta receptor would be beneficial. One of ordinary skill in the art will also appreciate that when using the compounds of the invention in the treatment of a specific disease that the compounds of the invention may be combined with various existing therapeutic agents used for that disease.

Other mammalian diseases and disorders which are treatable by administration of an invention compound alone, an invention combination, or a pharmaceutical composition comprising the compound or combination as defined below, may include: rheumatic diseases such as arthritis, inflammatory skin diseases such as psoriasis, eczema, atopic dermatitis, discoid lupus, contact dermatitis, bullous pemphigoid, vulgaris, and alopecia greata, fever (including rheumatic fever and fever associated with influenza and other viral infections), fibromyalgia, sleep disorders, common cold, dysmenorrhea, menstrual cramps, inflammatory bowel disease, Crohn's disease, emphysema, acute respiratory distress syndrome, asthma, bronchitis, chronic obstructive pulmonary disease, Alzheimer's disease, organ transplant toxicity, cachexia, allergic reactions, allergic contact hypersensitivity, cancer (such as solid tumor cancer including colon cancer, breast cancer, lung cancer and prostrate cancer; hematopoietic malignancies including leukemias and lymphomas; Hodgkin's disease; aplastic anemia, skin cancer and familiar adenomatous polyposis), tissue ulceration, peptic ulcers, gastritis, regional enteritis, ulcerative colitis, diverticulitis, recurrent gastrointestinal lesion, gastrointestinal bleeding, coagulation, anemia, synovitis, gout, ankylosing spondylitis, restenosis, periodontal disease, epidermolysis bullosa, osteoporosis, loosening of artificial joint implants, atherosclerosis (including atherosclerotic plaque rupture), aortic aneurysm (including abdominal aortic aneurysm and brain aortic aneurysm), periarteritis nodosa, congestive heart failure, myocardial infarction, stroke, cerebral ischemia, head trauma, spinal cord injury, neuralgia, neuro-degenerative disorders (acute and chronic), autoimmune disorders, Huntington's disease, Parkinson's disease, migraine, depression, peripheral neuropathy, pain (including low back and neck pain, headache, toothache, and neuropathic pain), gingivitis, cerebral amyloid angiopathy, nootropic or cognition enhancement, amyotrophic lateral sclerosis, multiple sclerosis, ocular angiogenesis, corneal injury, macular degeneration, conjunctivitis, abnormal wound healing, muscle or joint sprains or strains, tendonitis, skin disorders (such as psoriasis, eczema, scleroderma and dermatitis), myasthenia gravis, polymyositis, myositis, bursitis, burns, diabetes (including types I and II diabetes, diabetic retinopathy, neuropathy and nephropathy), tumor invasion, tumor growth, tumor metastasis, corneal scarring, scleritis, immunodeficiency diseases (such as AIDS in humans and FLV, FIV in cats), sepsis, premature labor, hypoprothrombinemia, hemophilia, thyroiditis, sarcoidosis, Behcet's syndrome, hypersensitivity, kidney disease, Rickettsial infections (such as Lyme disease, Erlichiosis), Protozoan diseases (such as malaria, giardia, coccidia), reproductive disorders (preferably in livestock), epilepsy, convulsions, and septic shock.

For the treatment of rheumatoid arthritis, the compounds of the present invention may be combined with agents such as TNF-α inhibitors such as (i) anti-TNF monoclonal antibodies such as adalimumab, which is known in the United States by the trade name HUMIRA® and infliximab, which is marketed in the United States under the trade name REMICADE® for the treatment of moderately to severely active Crohn's disease for reduction of signs and symptoms in patients who do not adequately respond to conventional therapies and treatment of patients with fistulizing Crohn's disease for the reduction in the number of draining enterocutaneous fistula(s); (ii) TNF receptor immunoglobulin molecules such as etanercept, which is marketed in the United States under the trade name Enbrel® for the treatment of rheumatoid arthritis, juvenile rheumatoid arthritis, and psoriatic arthritis; (iii) low dose methotrexate; (iv) lefunimide; (v) hydroxychloroquine; (vi) d-penicillamine; (vii) auranofin; (viii) or parenteral or oral gold.

The compounds of the invention can also be used in combination with existing therapeutic agents for the treatment of osteoarthritis. Suitable agents to be used in combination include standard non-steroidal anti-inflammatory agents (hereinafter NSAID's) such as piroxicam, diclofenac, propionic acids such as naproxen, flurbiprofen, fenoprofen, ketoprofen and ibuprofen, fenamates such as mefenamic acid, indomethacin, sulindac, apazone, pyrazolones such as phenylbutazone, salicylates such as aspirin, COX-2 inhibitors such as celecoxib, which is marketed in the United States under the trade name CELEBREX®, valdecoxib, which is marketed in the United States under the trade name BEXTRA®, parecoxib, etoricoxib, which is marketed in the United Kingdom under the trade name ARCOXIA®, and rofecoxib, which is marketed in the United States under the trade name VIOXX®, analgesics, and intraarticular therapies such as corticosteroids and hyaluronic acids such as hyalgan and synvisc.

As mentioned above, the invention compounds can also be used in combination with existing therapeutic agents for the prevention or treatment of arthritis, including osteoarthritis, joint inflammation, and joint pain. Suitable agents to be used in combination include standard non-steroidal anti-inflammatory agents (hereinafter NSAID's) such as piroxicam, diclofenac, propionic acids such as naproxen, flurbiprofen, fenoprofen, ketoprofen and ibuprofen, fenamates such as mefenamic acid, indomethacin, sulindac, apazone, pyrazolones such as phenylbutazone, salicylates such as aspirin, selective COX-2 inhibitors such as celecoxib, valdecoxib, parecoxib, rofecoxib, and the like, analgesics and intraarticular therapies such as corticosteroids and hyaluronic acids such as hyalgan and synvisc.

This invention also relates to a method of or a pharmaceutical composition for inhibiting joint cartilage damage and treating inflammatory processes and diseases comprising administering an invention compound to a mammal, including a human, cat, livestock or dog, wherein said joint cartilage damage and inflammatory processes and diseases are defined as above and said inhibitory compound is used in combination with one or more other therapeutically active agents under the following conditions:
A.) where a joint has become seriously inflamed as well as infected at the same time by bacteria, fungi, protozoa and/or virus, said inhibitory combination is administered in combination with one or more antibiotic, antifungal, antiprotozoal and/or antiviral therapeutic agents;
B.) where a multi-fold treatment of pain and inflammation is desired, said inhibitory combination is administered in combination with inhibitors of other mediators of inflammation, comprising one or more members independently selected from the group consisting essentially of:
(1) NSAIDs;
(2) H₁-receptor antagonists;
(3) kinin-B₁- and B₂-receptor antagonists;
(4) prostaglandin inhibitors selected from the group consisting of PGD-, PGF- PGI₂- and PGE-receptor antagonists;
(5) thromboxane A₂ (TXA₂-) inhibitors;
(6) 5-, 12- and 15-lipoxygenase inhibitors;
(7) leukotriene LTC₄-, LTD₄/LTE₄- and LTB₄-inhibitors;
(8) PAF-receptor antagonists;
(9) gold in the form of an aurothio group together with one or more hydrophilic groups;
(10) immunosuppressive agents selected from the group consisting of cyclosporine, azathioprine and methotrexate;
(11) anti-inflammatory glucocorticoids;
(12) penicillamine;
(13) hydroxychloroquine;
(14) anti-gout agents including colchicine; xanthine oxidase inhibitors including allopurinol; and uricosuric agents selected from probenecid, sulfinpyrazone and benzbromarone;
C. where older mammals are being treated for disease conditions, syndromes and symptoms found in geriatric mammals, said inhibitory combination is administered in combination with one or more members independently selected from the group consisting essentially of:
(1) cognitive therapeutics to counteract memory loss and impairment;
(2) anti-hypertensives and other cardiovascular drugs intended to offset the consequences of atherosclerosis, hypertension, myocardial ischemia, angina, congestive heart failure and myocardial infarction, selected from the group consisting of:
a. diuretics;
b. vasodilators;
c. β-adrenergic receptor antagonists;
d. angiotensin-II converting enzyme inhibitors (ACE-inhibitors), alone or optionally together with neutral endopeptidase inhibitors;
e. angiotensin II receptor antagonists;
f. renin inhibitors;
g. calcium channel blockers;
h. sympatholytic agents;
i. α₂-adrenergic agonists;
j. α-adrenergic receptor antagonists; and
k. HMG-CoA-reductase inhibitors (anti-hypercholesterolemics);
(3) antineoplastic agents selected from:
a. antimitotic drugs selected from:
i. vinca alkaloids selected from:
[1] vinblastine and
[2] vincristine;
(4) growth hormone secretagogues;
(5) strong analgesics;
(6) local and systemic anesthetics; and
(7) H₂-receptor antagonists, proton pump inhibitors and other gastroprotective agents.

The invention compounds may be administered in combination with inhibitors of other mediators of inflammation, comprising one or more members selected from the group consisting essentially of the classes of such inhibitors and examples thereof which include, matrix metalloproteinase inhibitors, aggrecanase inhibitors, TACE inhibitors, leukotriene receptor antagonists, IL-1 processing and release inhibitors, ILra, H₁-receptor antagonists; kinin-B₁- and B₂-receptor antagonists; prostaglandin inhibitors such as PGD-, PGF- PGI₂- and PGE-receptor antagonists; thromboxane A₂ (TXA2-) inhibitors; 5- and 12-lipoxygenase inhibitors; leukotriene LTC₄-, LTD₄/LTE₄- and LTB₄-inhibitors; PAF-receptor antagonists; MEK inhibitors; IKK inhibitors; MKK inhibitors; gold in the form of an aurothio group together with various hydrophilic groups; immunosuppressive agents, e.g., cyclosporine, azathioprine and methotrexate; anti-inflammatory glucocorticoids; penicillamine; hydroxychloroquine; anti-gout agents, e.g., colchicine, xanthine oxidase inhibitors, e.g., allopurinol and uricosuric agents, e.g., probenecid, sulfinpyrazone and benzbromarone.

Preferably, the invention compounds may be used in combination with a COX-2 selective inhibitor, more preferably celecoxib (e.g., CELEBREX®), valdecoxib (e.g., BEXTRA®), parecoxib, or rofecoxib (e.g., VIOXX®), or with compounds such as etanercept (e.g., ENBREL®), infliximab (e.g., REMICADE®), leflunomide, (e.g., ARAVA®) or methotrexate, and the like.

The invention compounds may also be used in combination with anticancer agents such as endostatin and angiostatin or cytotoxic drugs such as adriamycin, daunomycin, cis-platinum, etoposide, taxol, taxotere and alkaloids, such as vincristine and antimetabolites such as methotrexate.

The invention compounds may also be used in combination with anti-hypertensives and other cardiovascular drugs intended to offset the consequences of atherosclerosis, including hypertension, myocardial ischemia including angina, congestive heart failure and myocardial infarction, selected from vasodilators such as hydralazine, β-adrenergic receptor antagonists such as propranolol, calcium channel blockers such as nifedipine, (x₂-adrenergic agonists such as clonidine, α-adrenergic receptor antagonists such as prazosin and HMG-CoA-reductase inhibitors (anti-hypercholesterolemics) such as lovastatin or atorvastatin.

The invention compounds may also be administered in combination with one or more antibiotic, antifungal, antiprotozoal, antiviral or similar therapeutic agents.

The invention compounds may also be used in combination with CNS agents such as antidepressants (such as sertraline), anti-Parkinsonian drugs (such as L-dopa, requip, mirapex, MAOB inhibitors such as selegine and rasagiline, comP inhibitors such as Tasmar, A-2 inhibitors, dopamine reuptake inhibitors, NMDA antagonists, nicotine agonists, dopamine agonists and inhibitors of neuronal nitric oxide synthase) and anti-Alzheimer's drugs such as donepezil, tacrine, COX-2 inhibitors, propentofylline or metryfonate.

The invention compounds may also be used in combination with osteoporosis agents such as roloxifene, lasofoxifene, droloxifene or fosomax and immunosuppressant agents such as FK-506 and rapamycin.

The present invention also relates to the formulation of a compound of the present invention alone or with one or more other therapeutic agents which are to form the intended combination, including wherein said different drugs have varying half-lives, by creating controlled-release forms of said drugs with different release times which achieves relatively uniform dosing; or, in the case of non-human patients, a medicated feed dosage form in which said drugs used in the combination are present together in admixture in the feed composition. There is further provided in accordance with the present invention co-administration in which the combination of drugs is achieved by the simultaneous administration of said drugs to be given in combination; including co-administration by means of different dosage forms and routes of administration; the use of combinations in accordance with different but regular and continuous dosing schedules whereby desired plasma levels of said drugs involved are maintained in the patient being treated, even though the individual drugs making up said combination are not being administered to said patient simultaneously.

The invention method is useful in human and veterinary medicines for treating mammals suffering from one or more of the above-listed diseases and disorders. In humans, patients in need of treatment with an invention compound may be identified by a medical practitioner using conventional means. For example, patients at risk of having asymptomatic joint cartilage damage (e.g., osteoarthritis patients) may be identified clinically by assaying synovial fluid from an asymptomatic, at-risk mammal for the presence of breakdown products from the extracellular matrix (for example, proteoglycans, type II cartilage, or hydroxyproline), specialized X-ray techniques, or nuclear magnetic resonance imaging (“MRI”) techniques. Human asymptomatic persons at-risk for cartilage damage or osteoarthritis include elite athletes, laborers such as foundry workers, bus drivers, or coal miners, persons with above-normal C-reactive protein levels, and persons with a family history of osteoarthritis. Further, persons presenting clinically with joint stiffness, joint pain, loss of joint function, or joint inflammation may be examined for joint cartilage damage using the above methods.

It should be appreciated that the invention method can be employed prophylactically to prevent or inhibit the onset of joint inflammation, osteoarthritis, joint cartilage damage, or joint pain in a mammal. Patients who would benefit from prophylactic treatment include persons at risk for developing joint cartilage damage and persons who have developed joint cartilage damage but do not present clinically with secondary symptoms such as joint pain, joint stiffness, or in some cases, joint inflammation. These patients may be identified as described above.

The invention compounds are useful in human and veterinary medicines for alleviating joint pain, treating osteoarthritis, rheumatoid arthritis, joint inflammation, or inhibiting joint cartilage damage in a mammal, and for treating any other disease or disorder wherein joint inflammation or joint pain is a symptom or wherein joint cartilage damage is involved in the underlying pathology of the condition being treated.

All that is required to practice a method of this invention is to administer to a patient a compound of Formula I, or a pharmaceutically acceptable salt thereof, in a sufficiently nontoxic amount that is therapeutically effective for preventing, inhibiting, or reversing the condition being treated. The invention compound can be administered directly or as part of a pharmaceutical composition.

Pharmaceutical compositions include the following embodiments:

FORMULATION EMBODIMENT 1
Tablet Formulation:

| Ingredient | Amount (mg) |
|---|---|
| [2(S),3a(S),7a(S)]-1-methyl-2,3,3a,4,5,6,7,7a- | 25 |
| octahydroindole-2-carboxylic acid | |
| hydrochloride | |
| Lactose | 50 |
| Cornstarch (for mix) | 10 |
| Cornstarch (paste) | 10 |
| Magnesium stearate (1%) | 5 |
| Total | 100 |

[1(R),3a(R),7a(S)]-2-methyl-1,3,3 a,4,5,6,7,7a-octahydroisoindole-1-carboxylic acid hydrochloride, lactose, and cornstarch (for mix) are blended to uniformity. The cornstarch (for paste) is suspended in 200 mL of water and heated with stirring to form a paste. The paste is used to granulate the mixed powders. The wet granules are passed through a No. 8 hand screen and dried at 80° C. The dry granules are lubricated with the 1% magnesium stearate and pressed into a tablet. Such tablets can be administered to a human from one to four times a day for inhibiting joint cartilage damage or treating osteoarthritis.

### Formulation Embodiment 2

Coated Tablets:

The tablets of Formulation Embodiment 1 are coated in a customary manner with a coating of sucrose, potato starch, talc, tragacanth, and colorant.

### Formulation Embodiment 3

Capsules:

2 kg of [1(R),3a(R),7a(S)]-2-methyl-1,3,3a,4,5,6,7,7a-octahydroisoindole-1-carboxylic acid are filled into hard gelatin capsules in a customary manner such that each capsule contains 25 mg of [1(R),3a(R),7a(S)]-2-methyl-1,3,3a,4,5,6,7,7a-octahydroisoindole-1-carboxylic acid.

### Formulation Embodiment 4

Patch:

Ten milligrams of the compound of Compound Example F1 can be mixed with 1 mL of propylene glycol and 2 mg of acrylic-based polymer adhesive containing a resinous cross-linking agent. The mixture is applied to an impermeable backing (30 cm²) and applied to the upper back of a patient for sustained release treatment of joint cartilage damage or rheumatoid arthritis.

### Formulation Embodiment 5

Parenteral Solution:

In a solution of 700 mL of propylene glycol and 200 mL of water for injection can be added 20.0 g of the compound of Compound Example Q2. The mixture is stirred, and the pH is adjusted to 5.5 with hydrochloric acid. The volume is adjusted to 1000 mL with water for injection. The solution is sterilized, filled into 5.0 mL ampules, each containing 2.0 mL (40 mg of invention compound), and sealed under nitrogen. The solution is administered by injection to a patient suffering from osteoarthritis.

It should be appreciated that the compound of Formula (E) per se may be used in any of the additional embodiments described above by replacing the compound of Formula I in the embodiments with the compound of Formula (E).

Definitions:

The terms and phrases used herein are as defined below, as they otherwise occur in the specification or claims, or as they are commonly understood by one of ordinary skill in the related art.

As seen above, the groups of Formula I include “C₁-C₆ alkyl” groups. C₁-C₆ alkyl groups are straight and branched carbon chains having from 1 to 6 carbon atoms. Examples of C₁-C₆ alkyl groups include methyl, ethyl, 1-propyl, 2-propyl, 1-butyl, 2-butyl, 2,2-dimethylethyl, 1-pentyl, 2-pentyl, 2,2-dimethylpropyl, and 1-hexyl.

A substituted C₁-C₆ alkyl is a C₁-C₆ alkyl as defined above wherein the C₁-C₆ alkyl group is substituted with from 1 to 4 substituents independently selected from the substituent list above. Illustrative examples of substituted C₁-C₆ alkyl groups include CH₂OH, CF₂OH, CH₂C(CH₃)₂CO₂CH₃, CF₃, C(O)CF₃, C(O)—CH₃, (CH₂)₄—S—CH₃, CH(CO₂H)CH₂CH₂C(O)NMe₂, (CH₂)₅NH—C(O)—NH₂, CH₂—CH₂-C(H)-(4-fluorophenyl), CH(OCH₃)CH₂CH₃, CH₂SO₂NH₂, and CH(CH₃)CH₂CH₂OC(O)CH₃.

The term “C₂-C₆ alkenyl” means a straight or branched, unsubstituted hydrocarbon group having from 2 to 6 carbon atoms and 1 or 2 carbon-carbon double bonds, and include allenyl groups. Typical examples of C₂-C₆ alkenyl groups include ethenyl, 1-propen-1-yl, 1-propen-2-yl, 2-propen-1-yl, 1-buten-3-yl, 2-penten-2-yl, and 1-hexen-6-yl.

A substituted C₂-C₆ alkenyl is a C₂-C₆ alkenyl as defined above, which is substituted with from 1 to 4 substituents independently selected from the substituent list above. Illustrative examples of substituted C₂-C₆ alkenyl groups include C(H)═C(H)CH₂OH, CH═CF₂, CH₂C(H)=C(H)—(CH₂)₂CF₂OH, CH₂C(═CH₂)CO₂CH₃, C(H)═C(H)—CF₃, CH₂—CH₂-C(H)═C(H)—C(O)—CH₃, C(H)═C(CH₃)—S—CH₃, C(H)═C(H)—C(H)═C(CH₃)—CO₂Me, and C(H)=C═C(H)OC(O)CH₃.

The term “C₂-C₆ alkynyl” means a straight or branched, unsubstituted hydrocarbon group having from 2 to 6 carbon atoms and 1 or 2 carbon-carbon triple bonds. Typical examples of C₂-C₆ alkynyl groups include ethenyl, 1-propyn-1-yl, 1-propyn-3-yl, 1-butyn-3-yl, 2-pentyn-1-yl, and 1-hexyn-6-yl.

A substituted C₂-C₆ alkynyl is a C₂-C₆ alkynyl as defined above, which is substituted with from 1 to 4 substituents independently selected from the substituent list above. Illustrative examples of substituted C₂-C₆ alkynyl groups include C≡CCH₂OH, C—CF, CH₂C═C—(CH₂)₂CF₂OH, C≡C—CH₂CO₂CH₃, CH₂C≡C—CF₃, CH₂—CH₂-C≡C—C(O)—CH₃, C≡C—S—CH₃, and C≡C—C(O)OC(O)CH₃.

The phrase “2- to 6-membered heteroalkyl” means a saturated radical chain that is straight or branched and contains from 1 to 5 carbon atoms and 1 heteroatom selected from O, S, S(O), S(O)₂, N(H), and N(C₁-C₆ alkyl). Illustrative examples of 2- to 6-membered heteroalkyl include OCH₃, CH₃CH₂O, CH₃C(CH₃)HS, and CH₂CH₂N(H)CH₂CH₂CH₃.

A substituted 2- to 6-membered heteroalkyl is a 2- to 6-membered heteroalkyl as defined above, which is substituted with from 1 to 4 substituents independently selected from the list above. Illustrative examples of substituted 2-to 6-membered heteroalkyl groups include OCF₃, CH₃C(O)O, CH₃C(CH₃)HS, and CH₂CH₂N(CH₂CH₂CH₃)CH₂C(OH)HCH₃.

The phrase “2- to 6-membered heteroalkenyl” means a radical chain that is straight or branched and contains from 1 to 5 carbon atoms and 1 heteroatom selected from O, S, S(O), S(O)₂, N(H), and N(C₁-C₆ alkyl), and one carbon-carbon or carbon-nitrogen double bond. Illustrative examples of 2- to 6-membered heteroalkenyl include N═CH₂, CH═CHOCH₃, and CH₂C(H)=C(H)CH₂N(H)CH₃.

A substituted 2- to 6-membered heteroalkenyl is a 2- to 6-membered heteroalkenyl, as defined above, which is substituted with from 1 to 4 substituents independently selected from the substituent list above. Illustrative examples of substituted 2- to 6-membered heteroalkenyl include N═C(OH)H, CH═CHOCF₃, and CH₂C(H)═C(H)C(O)N(H)CH₃.

The term “C₃-C₇ cycloalkyl” means an unsubstituted, saturated cyclic hydrocarbon group having from 3 to 7 carbon atoms. The group C₃-C₇ cycloalkyl includes cyclopropyl, cyclobutyl, cyclopentyl, cyclohexyl, and cycloheptyl.

A substituted C₃-C₇ cycloalkyl is a C₃-C₇ cycloalkyl as defined above, which is substituted with from 1 to 4 substituents independently selected from the substituent list above. Illustrative examples of substituted C₃-C₇ cycloalkyl groups include 1-hydroxy-cyclopropyl, cyclobutanon-3-yl, 3-(3-phenyl-ureido)-cyclopent-1-yl, and 4-carboxy-cyclohexyl.

The term “C₃-C₇ cycloalkenyl” means an unsubstituted cyclic hydrocarbon group having from 3 to 7 carbon atoms and 1 carbon-carbon double bond. The group C₃-C₇ cycloalkenyl includes cyclopropenyl, cyclobutenyl, cyclopentenyl, cyclohexenyl, and cycloheptenyl.

A substituted C₃-C₇ cycloalkenyl is a C₃-C₇ cycloalkenyl as defined above, which is substituted with from 1 to 4 substituents independently selected from the substituent list above. Illustrative examples of substituted C₃-C₇ cycloalkenyl groups include 1-hydroxy-cyclopropen-2-yl, cyclobutenon-3-yl, 3-(3-phenyl-ureido)-cyclopenten-1-yl, and 4-carboxy-cyclohexenyl.

The phrase “C₇-C₁₀ bicycloalkyl” means a saturated fused or bridged bicyclic carbon ring system which is (i) a cyclopentyl or cyclohexyl ring fused to another cyclopentyl or cyclohexyl ring to give a 5,5-, 5,6-, or 6,6-fused bicyclic carbocyclic group of from 8 to 10 carbon atoms or (ii) a bridged bicyclic group of from 7 to 10 carbon atoms. Illustrative examples of fused bicycloalkyl groups of from 8 to 10 carbon atoms include bicyclo[3.3.0]octyl, bicyclo[4.3.0]nonyl, and bicyclo[4.4.0]decyl. Illustrative examples of bridged bicyclic groups of from 7 to 10 carbon atoms include bicyclo[2.2.1]heptyl, bicyclo[2.2.2.]octyl, bicyclo[3.2.1]octyl, and bicyclo[4.3.1]decyl.

A substituted C₇-C₁₀ bicycloalkyl is a C₇-C₁₀ bicycloalkyl, as defined above, substituted with from 1 to 4 substituents independently selected from the substituent list above. Illustrative examples of substituted fused bicycloalkyl groups of from 8 to 10 carbon atoms include 2-oxo-bicyclo[3.3.0]octan-3-yl, 1-fluoro-bicyclo[4.3.0]nonyl, and 8-hydroxy-1-methyl-bicyclo[4.4.0]decyl. Illustrative examples of substituted bridged bicyclic groups of from 7 to 10 carbon atoms include 1-hydroxy-bicyclo[2.2.1]heptyl, 2-oxo-3-methyl-bicyclo[2.2.2.]octyl, and 1-carboxy-8-oxo-bicyclo[3.2.1]octyl.

The phrase “3- to 7-membered heterocycloalkyl” means a saturated monocyclic ring containing from 1 to 6 carbon atoms and 1 or 2 heteroatoms independently selected from 1 O, 1 S, 1 S(O), 1 S(O)₂, 1 N, 2 N(H), and 2 N(C₁-C₆ alkyl). Illustrative examples of 3- to 7-membered heterocycloalkyl include aziridinyl, 2-oxo-2-thia-cyclobutyl, pyrrolidinyl, piperidinyl, morpholinyl, piperazinyl, and 4-oxacycloheptyl.

A substituted 3- to 7-membered heterocycloalkyl is a 3- to 7-membered heterocycloalkyl, as defined above, substituted with from 1 to 4 substituents independently selected from the substituent list above. Illustrative examples of substituted 3- to 7-membered heterocycloalkyl include 1-(2-ethanyol)-aziridinyl, 2,2-dioxo-3-methyl-2-thia-cyclobutyl, 2-oxo-pyrrolidinyl, 1-acetyl-piperidinyl, 3,3-dimethylmorpholinyl, 4-benzyl-piperazinyl, and 2-thienyl-4-oxa-cycloheptyl.

The phrase “7- to 10-membered heterobicycloalkyl” means a saturated fused or bridged bicyclic ring system containing from 5 to 9 carbon atoms and 1 or 2 heteroatoms independently selected from 1 O, 1 S, 1 S(O), 1 S(O)₂, 1N, 2 N(H), and 2 N(C₁-C₆ alkyl), which is (i) a 5- or 6-membered ring fused to another 5- or 6-membered ring to give a 5,5-, 5,6-, or 6,6-fused heterobicyclic group of from 8 to 10 atoms or (ii) a bridged bicyclic group of from 7 to 10 atoms. Illustrative examples of fused heterobicycloalkyl groups of from 8 to 10 atoms include 1-azabicyclo[3.3.0]octyl, 5-oxabicyclo[4.3.0]nonyl, and 2,2-dioxo-2-thiabicyclo[4.4.0]decyl. Illustrative examples of bridged bicyclic groups of from 7 to 10 atoms include 7-oxabicyclo[2.2.1]heptyl, 1-azabicyclo[2.2.2.]octyl, and 10-oxo-10-thiabicyclo[4.3.1]decyl.

A substituted 7- to 10-membered heterobicycloalkyl is a 7- to 10-membered heterobicycloalkyl, as defined above, substituted with from 1 to 4 substituents independently selected from the substituent list above. Illustrative examples of substituted fused heterobicycloalkyl groups of from 8 to 10 atoms include 2-oxo-1-azabicyclo[3.3.0]octyl, 1-methyl-S-oxabicyclo[4.3.0]nonyl, and 1-phenyl-2,2-dioxo-2-thiabicyclo[4.4.0]decyl. Illustrative examples of substituted bridged heterobicyclic groups of from 7 to 10 atoms include 2-(3-fluorophenyl)-7-oxabicyclo[2.2.1]heptyl, 2-oxo-3-methyl-1-azabicyclo[2.2.2.]octyl, and 1-tetrazol-5-yl-10-oxo-10-thiabicyclo[4.3.1]decyl.

The phrase “5- and 6-membered heteroaryl” means a 5-membered, monocyclic heteroaryl having carbon atoms and from 1 to 4 heteroatoms independently selected from 1 O, 1 S, 1 N(H), 1N(C₁-C₆ alkyl), and 4 N, and a 6-membered, monocyclic heteroaryl having carbon atoms and 1 or 2 heteroatoms selected from 2 N, and wherein:
(i) The phrase “5-membered, monocyclic heteroaryl” means a 5-membered, monocyclic, aromatic ring group as defined above having carbon atoms and from 1 to 4 heteroatoms selected from 1 O, 1 S, 1 N(H), 1 N(C₁-C₆ alkyl), and 4 N. Illustrative examples of a 5-membered, monocyclic heteroaryl include thiophen-2-yl, furan-2-yl, pyrrol-3-yl, pyrrol-1-yl, imidazol-4-yl, isoxazol-3-yl, oxazol-2-yl, thiazol-4-yl, tetrazol-1-yl, 1,2,4-oxadiazol-3-yl, 1,2,4-triazol-1-yl, and pyrazol-3-yl; and
(ii) The phrase “6-membered, monocyclic heteroaryl” means a 6-membered, monocyclic, aromatic ring group as defined above having carbon atoms and 1 or 2 N. Illustrative examples of a 6-membered, monocyclic heteroaryl include pyridin-2-yl, pyridin-4-yl, pyrimidin-2-yl, pyridazin-4-yl, and pyrazin-2-yl.

The phrase “8- to 10-membered heterobiaryl” means an 8-membered, 5,5-fused bicyclic heteroaryl, a 9-membered, 6,5-fused bicyclic heteroaryl, or a 10-membered, 6,6-fused bicyclic heteroaryl, having carbon atoms and from 1 to 4 heteroatoms independently selected from 1 O, 1 S, 1 N(H), 1 N(C₁-C₆ alkyl), and 4 N, wherein at least one of the 2 fused rings is aromatic, and wherein when the O and S atoms both are present, the O and S atoms are not bonded to each other, which are as defined below:
(i) The phrase “8-membered, 5,5-fused bicyclic heteroaryl” means a an 8-membered aromatic, fused-bicyclic ring group as defined above having carbon atoms and from 1 to 4 heteroatoms selected from 1 O, 1 S, 1 N(H), 1 N(C₁-C₆ alkyl), and 4 N. Illustrative examples of an 8-membered, fused-bicyclic heteroaryl include 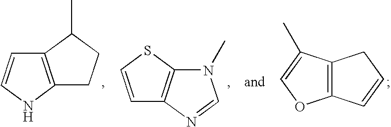
(ii) The phrase “9-membered, 6,5-fused bicyclic heteroaryl” means a 9-membered aromatic, fused-bicyclic ring group as defined above having carbon atoms and from 1 to 4 heteroatoms selected from 1 O, 1 S, 1 N(H), 1 N(C₁-C₆ alkyl), and 4 N. Illustrative examples of a 9-membered, fused-bicyclic heteroaryl include indol-2-yl, indol-6-yl, iso-indol-2-yl, benzimidazol-2-yl, benzimidazol-1-yl, benztriazol-1-yl, benztriazol-5-yl, benzoxazol-2-yl, benzothiophen-5-yl, and benzofuran-3-yl; and
(iii) The phrase “10-membered, 6,5-fused bicyclic heteroaryl” means a 10-membered aromatic, fused-bicyclic ring group as defined above having carbon atoms and from 1 to 4 heteroatoms selected from 1 O, 1 S, 1 N(H), 1 N(C₁-C₆ alkyl), and 4 N. Illustrative examples of a 10-membered, fused-bicyclic heteroaryl include quinolin-2-yl, isoquinolin-7-yl, and benzopyrimidin-2-yl.

A substituted 5- or 6-membered heteroaryl and a substituted 8- to 10-membered heterobiaryl are a 5- or 6-membered heteroaryl, as defined above, and an 8- to 10-membered heterobiaryl, as defined above, respectively, which are substituted on a carbon (CH) atom, and/or nitrogen [N(H)] atom in the case of 5-member heteroaryl and 8- to 10-membered heterobiaryl, with from 1 to 4 substituents independently selected from the list above.

Illustrative examples of substituted 5-membered, monocyclic heteroaryl groups include 2-hydroxy-oxoazol-4-yl, 5-chloro-thiophen-2-yl, 1-methylimidazol-5-yl, 1-propyl-pyrrol-2-yl, 1-acetyl-pyrazol-4-yl, 1-methyl-1,2,4-triazol-3-yl, and 2-hexyl-tetrazol-5-yl.

Illustrative examples of substituted 6-membered, monocyclic heteroaryl groups include 4-acetyl-pyridin-2-yl, 3-fluoro-pyridin-4-yl, 5-carboxy-pyrimidin-2-yl, 6-tertiary butyl-pyridazin-4-yl, and 5-hdyroxymethyl-pyrazin-2-yl.

Illustrative examples of substituted 8-membered, 5,5-fused bicyclic heteroaryl include:

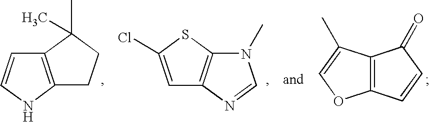

Illustrative examples of substituted 9-membered, 5,6-fused bicyclic heteroaryl include 3-(2-aminomethyl)-indol-2-yl, 2-carboxy-indol-6-yl, 1-(methanesulfonyl)-iso-indol-2-yl, 5-trifluorometyl-6,7-difluoro-4-hydroxymethyl-benzimidazol-2-yl, 4-(3-methylureido)-2-cyano-benzimidazol-1-yl, 1-methylbenzimidazol-6-yl, 1-acetylbenztriazol-7-yl, 1-methanesulfonyl-indol-3-yl, 1-cyano-6-aza-indol-5-yl, and 1-(2,6-dichlorophenylmethyl)-benzpyrazol-3-yl.

Illustrative examples of substituted 10-membered, 6,6-fused bicyclic heteroaryl include 5,7-dichloro-quinolin-2-yl, isoquinolin-7-yl-1-carboxylic acid ethyl ester, and 3-bromo-benzopyrimidin-2-yl.

Terms such as, for example, “C₁-C₆ alkyl-(G)ₘ”, “C₁-C₆ alkyl-(L)ₘ”, and “C₁-C₆ alkyl-(Q)ₘ”, mean, in this example, a C₁-C₆ alkyl, as defined above, bonded directly when m is 0, or bonded through a group G, L, or Q, respectively, when m is 1. Similarly, for example, the term “phenyl-(G)ₘ” means a phenyl bonded directly when m is 0 or bonded through a group G when m is 1.

The term “C₁-C₈ alkylenyl” means a saturated hydrocarbon diradical that is straight or branched and has from 1 to 8 carbon atoms. C₁-C₈ alkylenyl having from 2 to 8 carbon atoms may optionally independently contain one carbon-carbon double bond. Illustrative examples of C₁-C₈ alkylenyl include CH₂, CH₂CH₂, C(CH₃)H, C(H)(CH₃)CH₂CH₂, and CH₂C(H)═C(H)CH₂CH₂CH₂CH₂CH₂.

A substituted C₁-C₈ alkylenyl is a C₁-C₈ alkylenyl, as defined above, substituted with from 1 to 4 substituents independently selected from the substituent list above. Illustrative examples of substituted C₁-C₈ alkylenyl include CH(OH), CH₂CH(CF₃), C(CO₂H)H, C(H)(CH₃)C(O)CH₂, and CH₂C(H)═C(H)CH₂CH₂C(NH₂)HCH₂CH₂.

The phrase “2- to 8-membered heteroalkylenyl” means a saturated diradical chain that is straight or branched and contains from 1 to 7 carbon atoms and 1 heteroatom selected from O, S, S(O), S(O)₂, N(H), and N(C₁-C₆ alkyl). 2- to 8-membered heteroalkylenyl, having from 2 to 8 chain atoms, may optionally independently contain one carbon-carbon or one carbon-nitrogen double bond. Illustrative examples of 2- to 8-membered heteroalkylenyl include OCH₂, CH₂CH₂O, C(CH₃)HS, and CH₂C(H)═C(H)CH₂N(H)CH₂CH₂CH₂.

A substituted 2- to 8-membered heteroalkylenyl is a 2- to 8-membered heteroalkylenyl, as defined above, substituted with from 1 to 4 substituents independently selected from the substituent list above. Illustrative examples of substituted 2- to 8-membered heteroalkylenyl include OCF₂, CH₂C(O)O, C(CH₃)OHS(O), and CH₂C(H)═NCH₂CH₂N(OH)CH₂CH₂.

Terms such as “(C₁-C₈ alkylenyl)ₘ” and “(2- to 8-membered heteroalkylenyl)ₘ”, mean, in this example, the C₁-C₈ alkylenyl, as defined above, or the 2- to 8-membered heteroalkylenyl, as defined above, respectively is absent when m is 0, or is present when m is 1.

It should be appreciated that the symbol “ [symbol] ” in front of a bond from a structure indicates that the bond is a radical point of attachment in the structure.

Preferred substituents for substituted phenyl, substituted naphthyl (i.e., substituted 1-naphthyl or substituted 2-naphthyl), and preferred substituents at carbon atoms for substituted 5-membered, monocyclic heteroaryl, substituted 6-membered, monocyclic heteroaryl, and substituted 9- or 10-membered, fused-bicyclic heteroaryl are C₁-C₄ alkyl, halo, OH, O—C₁-C₄ alkyl, 1,2-methylenedioxy, CN, NO₂, N₃, NH₂, N(H)CH₃, N(CH₃)₂, C(O)CH₃, OC(O)—C₁-C₄ alkyl, C(O)—H, CO₂H, CO₂—(C₁-C₄ alkyl), C(O)—N(H)OH, C(O)NH₂, C(O)NHMe, C(O)N(Me)₂, NHC(O)CH₃, N(H)C(O)NH₂, SH, S—C₁-C₄ alkyl, C≡CH, C(═NOH)—H, C(═NOH)—CH₃, CH₂OH, CH₂NH₂, CH₂N(H)CH₃, CH₂N(CH₃)₂, C(H)F—OH, CF₂—OH, S(O)₂NH₂, S(O)₂N(H)CH₃, S(O)₂N(CH₃)₂, S(O)—CH₃, S(O)₂CH₃, S(O)₂CF₃, or NHS(O)₂CH₃.

Especially preferred substituents are 1,2-methylenedioxy, methoxy, ethoxy, —O—C(O)CH₃, carboxy, carbomethoxy, and carboethoxy.

It should be appreciated that the groups heteroaryl or heterocycloalkyl may not contain two ring atoms bonded to each other which atoms are oxygen and/or sulfur atoms.

The term “oxo” means=O. Oxo is attached at a carbon atom unless otherwise noted. Oxo, together with the carbon atom to which it is attached forms a carbonyl group (i.e., C═O).

The term “heteroatom” includes O, S, S(O), S(O)₂, N,N(H), and N(C₁-C₆ alkyl).

The term “halo” includes fluoro, chloro, bromo, and iodo.

The term “amino” means NH₂.

It should be appreciated that a 5- or 6-membered heteroaryl or an 8- to 10-membered heterobiaryl includes groups such as benzimidazolyl, benzofuranyl, benzofurazanyl, 2H-1-benzopyranyl, benzothiadiazine, benzothiazinyl, benzothiazolyl, benzothiophenyl, benzoxazolyl, chromanyl, cinnolinyl, furazanyl, furopyridinyl, indazolyl, indolinyl, indolizinyl, indolyl, 3H-indolyl, isoindolyl, isoquinolinyl, isothiazolyl, isoxazolyl, naphthyridinyl, oxadiazolyl, oxazolyl, phthalazinyl, pteridinyl, purinyl, pyrazinyl, pyridazinyl, pyridinyl, pyrimidinyl, pyrazolyl, pyrrolyl, quinazolinyl, quinolinyl, quinoxalinyl, tetrazolyl, thiazolyl, thiadiazolyl, thienyl, triazinyl, triazolyl, benzofuran, isobenzofuran, benzothiofuran, isobenzothiofuran, indole, indolenine, 2-isobenzazole, 1,5-pyrindine, pyrano[3,4-b]-pyrrole, isoindazole, indoxazine, benzoxazole, anthranil, benzopyran, coumarin, chromone, isocoumarin, 2,3-benzopyrone, quinoline, isoquinoline, cinnoline, quinazoline, naphthyridine, pyrido[3,4-b]-pyridine, pyrido[3,2-b]-pyridine, pyrido[4,3-b]pyridine, and benzoxazine, wherein said group may be optionally substituted on any of the ring carbon atom or nitrogen atom capable of forming an additional bond as described above. The foregoing groups, as derived from the compounds listed above, can be C-attached or N-attached where such is possible. For example, a group derived from pyrrole can be pyrrol-1-yl (N-attached) or pyrrol4-yl (C-attached).

It should be appreciated that a 5-membered heteroarylenyl includes groups such as isothiazoldiyl, isoxazoldiyl, oxadiazoldiyl, oxazoldiyl, pyrazoldiyl, pyrroldiyl, tetrazoldiyl, thiazoldiyl, thiadiazoldiyl, thiendiyl, triazindiyl, triazoldiyl, and the like, wherein said group may be optionally substituted on any of the ring carbon atom or nitrogen atom capable of forming an additional bond as described above. The foregoing groups, as derived from the compounds listed above, can be C-attached or N-attached where such is possible. For example, a group derived from pyrrole can be pyrrol-1-yl (N-attached) or pyrrol4-yl (C-attached).

It should be appreciated that tautomeric forms (i.e., oxo forms) of substituted 5- or 6-membered heteroaryl or an 8- to 10-membered heterobiaryl groups bearing a hydroxy substituent on a carbon atom are included in the present invention.

It should be appreciated that in another embodiment, the invention compounds may further comprise compounds of Formula I wherein at least one indanyl, pentalenyl, indenyl, azulenyl, fluorenyl, or tetrahydronaphthyl group has been inserted in place of a phenyl or naphthyl group defined above for Formula I.

It should be appreciated that the invention compounds further comprise compounds of Formula I which are substituted with from 1 to 6 substituents, wherein the substituent(s) is selected from a group containing every chemically and pharmaceutically suitable substituent.

The phrase “chemically and pharmaceutically suitable substituent” is intended to mean a chemically and pharmaceutically acceptable functional group or moiety that does not negate the inhibitory activity of the inventive compounds or impart a degree of toxicity that would make the resulting substituted compound unsuitable for use as a pharmaceutical or veterinary agent. Such suitable substituents include those recited above for Formula I and those which may be routinely selected by those skilled in the art. Illustrative examples of suitable substituents include, but are not limited to halo groups, perfluoroalkyl groups, perfluoroalkoxy groups, alkyl groups, hydroxy groups, oxo groups, mercapto groups, alkylthio groups, alkoxy groups, aryl or heteroaryl groups, aryloxy or heteroaryloxy groups, aralkyl or heteroaralkyl groups, aralkoxy or heteroaralkoxy groups, carboxy groups, amino groups, alkyl- and dialkylamino groups, carbamoyl groups, alkylcarbonyl groups, alkoxycarbonyl groups, alkylaminocarbonyl groups dialkylamino carbonyl groups, arylcarbonyl groups, aryloxycarbonyl groups, alkylsulfonyl groups, an arylsulfonyl groups and the like.

The term “(E)” means entgegen, and designates that the conformation about the double bond to which the term refers is the conformation having the two higher ranking substituent groups, as determined according to the Cahn-Ingold-Prelog ranking system, on opposite sides of the double bond. An (E) double bond is illustrated below by the compound of Formula (W) 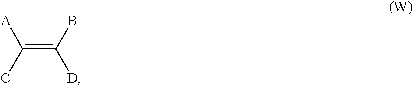
wherein the two higher-ranking substituents are groups A and D.

The term “(Z)” means zusammen, and designates that the conformation about the double bond to which the term refers is the conformation having the two higher ranking substituent groups, as determined according to the Cahn-Ingold-Prelog ranking system, on the same side of the double bond. A (Z) double bond is illustrated below by the compound of Formula (X) 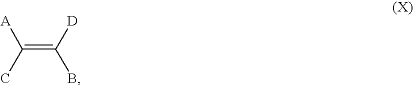
wherein the two higher-ranking substituents are groups A and D.

In a compound of Formula I, or a pharmaceutically acceptable salt thereof, it should be appreciated that in any (C₁-C₆ alkyl)₂-N group, the C₁-C₆ alkyl groups may be optionally taken together with the nitrogen atom to which they are attached to form a 5- or 6-membered heterocycloalkyl.

It should be appreciated that each group and each substituent recited above is independently selected unless otherwise indicated.

It should be appreciated that when reference is made to only one stereoisomer of a [c]-fused bicyclic proline derivative, what is meant is that the stereoisomer is substantially free from any other stereoisomer of the compound.

A stereoisomer of an invention compound which is substantially free of any other stereoisomer of the compound is a stereoisomer that does not contain more than 5% of any other stereoisomer of the compound. Preferably, substantially free means less than 3% of any other stereoisomer of the compound. More preferably, substantially free means less than 2% of any other stereoisomer of the compound. Still more preferably, substantially free means less than 1% of any other stereoisomer of the compound. Still more preferably, substantially free means less than 0.6% of any other stereoisomer of the compound. Still more preferably, substantially free means less than 0.5% of any other stereoisomer of the compound. Still more preferably, substantially free means less than 0.3% of any other stereoisomer of the compound. Still more preferably, substantially free means less than 0.2% of any other stereoisomer of the compound. Still more preferably, substantially free means less than 0.1% of any other stereoisomer of the compound. Still more preferably, substantially free means less than 0.05% of any other stereoisomer of the compound. Still more preferably, substantially free means less than 0.02% of any other stereoisomer of the compound. Still more preferably, substantially free means less than 0.01% of any other stereoisomer of the compound. Still more preferably, substantially free means less than 0.005% of any other stereoisomer of the compound.

For illustration purposes, the compound named [1(R), 3a(R), 7a(S)]-2-methyl-octahydroisoindole-1-carboxylic acid may also be known by the names [1(R), 3a(R), 7a(S)]-2-methyl-1,3,3a,4,5,6,7,7a-octahydroisoindol-1-carboxylic acid, and [1(R), 6(S), 7(R)]-8-methyl-8-azabicyclo[4.3.0]nonane-7-carboxylic acid, and has the structure drawn below:

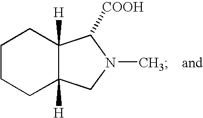

For illustration purposes, the compound named [1(R), 2(R), 5(R)]-3-aza-6-oxabicyclo[4.3.0]nonane-2-carboxylic acid may also be known by the names [4a(R), 5(R), 7a(R)]-octahydro-pyrano[2,3-c]pyrrole-5-carboxylic acid, and [1(R), 3a(R), 7a(R)]-octahydro-4-oxa-isoindole-1-carboxylic acid. The compound named [1(R), 2(R), 5(R)]-3-aza-6-oxabicyclo[4.3.0]nonane-2-carboxylic acid has the structure drawn below:

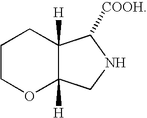

It should be appreciated that the 1,3,3a,4,5,6,7,7a-octahydroisoindole and octahydro-pyrano[2,3-c]pyrrole ring systems employ the following numbering schemes: 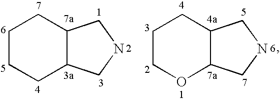
respectively.

For illustrative purposes, it should be appreciated that a [c]-fused bicyclic proline is a bicyclic derivative of proline that is fused as shown below:

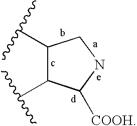

The term “admixture” means the state of being mixed.

Gabapentin is marketed under the tradename NEURONTIN® in the United States by Pfizer, Inc. for the treatment of epilepsy and has the structure drawn below:

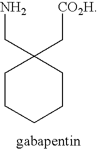

A selective inhibitor of COX-2 means a compound that inhibits COX-2 selectively versus COX-1 such that a ratio of IC₅₀ for a compound with COX-1 divided by a ratio of IC₅₀ for the compound with COX-2 is greater than, or equal to, 5, where the ratios are determined in one or more assays. All that is required to determine whether a compound is a selective COX-2 inhibitor is to assay a compound in one of a number of well know assays in the art.

The term “NSAID” is an acronym for the phrase “nonsteroidal anti-inflammatory drug”, which means any compound which inhibits cyclooxygenase-1 (“COX-1”) and cyclooxygenase-2. Most NSAIDs fall within one of the following five structural classes: (1) propionic acid derivatives, such as ibuprofen, naproxen, naprosyn, diclofenac, and ketoprofen; (2) acetic acid derivatives, such as tolmetin and sulindac; (3) fenamic acid derivatives, such as mefenamic acid and meclofenamic acid; (4) biphenylcarboxylic acid derivatives, such as diflunisal and flufenisal; and (5) oxicams, such as piroxim, peroxicam, sudoxicam, and isoxicam. Other useful NSAIDs include aspirin, acetominophen, indomethacin, and phenylbutazone. Selective inhibitors of cyclooxygenase-2 as described above may be considered to be NSAIDs also.

The phrase “tertiary organic amine” means a trisubstituted nitrogen group wherein the 3 substituents are independently selected from C₁-C₆ alkyl, C₃-C₆ cycloalkyl, benzyl, or wherein two of the substituents are taken together with the nitrogen atom to which they are bonded to form a 5- or 6-membered, monocyclic heterocycle containing one nitrogen atom and carbon atoms, and the third substituent is selected from C₁-C₆ alkyl and benzyl, or wherein the three substituents are taken together with the nitrogen atom to which they are bonded to form a 7- to 12-membered bicyclic heterocycle containing 1 or 2 nitrogen atoms and carbon atoms, and optionally a C═N double bond when 2 nitrogen atoms are present. Illustrative examples of tertiary organic amine include triethylamine, diisopropylethylamine, benzyl diethylamino, dicyclohexylmethyl-amine, 1,8-diazabicycle[5.4.0]undec-7-ene (DBU), 1,4-diazabicyclo[2.2.2]octane (TED), and 1,5-diazabicycle[4.3.0]non-5-ene.

The term “HPLC” means high performance liquid chromatography.

It should be appreciated that the terms “uses”, “utilizes”, and “employs”, and their derivatives thereof, are used interchangeably when describing an embodiment of the present invention.

It should be appreciated that the term “about,” when employed to modify a value in an expression of a range between two values or between and inclusive of two values, means plus or minus 20% of the value being modified. For illustration purposes, the phrase “from 4.0 to about 10” means from 3.9500 to 10 plus or minus 2, and thus means from 3.95 to 8, 9, 10, 11, or 12, inclusively. Increasingly preferred for “about” is plus or minus 15%, 10%, or 5%.

It should be further appreciated that when reference is made herein to a [c]-fused bicyclic proline derivative; an invention compound; or a compound mixture, drug, active substance, active component, and the like, what is being referred to includes a compound of Formula I, or a pharmaceutically acceptable salt thereof, or a solvates thereof, or an isotopically-labelled isomer thereof, or a tautomer thereof, or a polymorph thereof, and the like unless otherwise specified.

The term “drug” and the phrase “invention compound” are synonymous with the phrase “active ingredient” and includes an invention compound of Formula I, an invention compound of Formula (E), a compound mixture, or combination, or any of the other therapeutic agents described herein that may be used in combination with the invention compound, compound mixture, or combination in accordance with an invention method.

The term “IC₅₀” means the concentration of a drug, including an invention compound, or a pharmaceutically acceptable salt thereof, that is sufficient to inhibit 50% of the activity being measured.

The term “ED₄₀” means the dose of a drug, including an invention compound, or a pharmaceutically acceptable salt thereof, that is sufficient to prevent or inhibit joint cartilage damage or prevent or treat a disease or disorder listed above, in at least 40% of the patients being treated.

The term “patient” means a mammal. The methods of the present invention are useful as pharmaceuticals and veterinarian medicines for treating mammals, particularly humans, companion animals, and livestock animals. A preferred patient is a human. Other preferred patients are dogs, cats, cows, horses, and pigs.

For the purposes of this invention, the term “mammal” includes humans, companion animals such as cats and dogs, livestock animals such as horses, cows, pigs, goats, and sheep, and laboratory animals such as guinea pigs, rabbits, rats, mice, hamsters, and monkeys, and transgenic variants thereof. Preferred mammals are human, rat, mouse, rabbit, and dog. More preferred mammal is a human.

The phrase “companion animals” includes dogs, cats, rabbits, hamsters, monkeys, horses, and other household or barnyard pets.

The phrase “livestock animals” as used herein refers to domesticated quadrupeds, which includes those being raised for meat and various byproducts, e.g., a bovine animal including cattle and other members of the genus Bos, a porcine animal including domestic swine and other members of the genus Sus, an ovine animal including sheep and other members of the genus Ovis, domestic goats and other members of the genus Capra; domesticated quadrupeds being raised for specialized tasks such as use as a beast of burden, e.g., an equine animal including domestic horses and other members of the family Equidae, genus Equus, or for searching and sentinel duty, e.g., a canine animal including domestic dogs and other members of the genus Canis; and domesticated quadrupeds being raised primarily for recreational purposes, e.g., members of Equus and Canis, as well as a feline animal including domestic cats and other members of the family Felidae, genus Felis.

It should be appreciated that infectious arthritis may be caused by bacterial infections such as Lyme disease or gonorrhea, viral infections, or infections by fungi.

It should be appreciated that osteoarthritis is itself a noninflammatory condition that may be present for years in a patient before any manifest symptoms such as joint stiffness or swelling, diminishment of joint movement or function, or joint pain are appreciated by the patient.

The phrase “joint cartilage damage” means a disorder of hyaline cartilage and subchondral bone characterized by hypertrophy of tissues in and around the involved joints, which may or may not be accompanied by deterioration of hyaline cartilage surface.

The phrase “inhibiting joint cartilage damage” means the therapeutic effect of an invention compound, compound mixture, or combination that prevents the initiation of, or inhibits the progress, prevents further progress, or reverses progression, in part or in whole, of a disease pathology or any one or more symptoms of a related disease or disorder that is appreciated, suspected, or expected.

Disease pathology of joint cartilage damage related to osteoarthritis can include damage to cartilage or subchondral bone in a joint as described above. Symptoms of joint cartilage damage related to osteoarthritis may be absent for years in a patient, but, when present, can include joint stiffness, diminishment of joint movement or function, or joint pain.

Disease pathology of joint cartilage damage related to rheumatoid arthritis can include damage to cartilage or subchondral bone in a joint as described above. Symptoms of joint cartilage damage related to rheumatoid arthritis are frequently present and can include joint stiffness, diminishment of joint movement or function, or joint pain. Rheumatoid arthritis patients also typically have joint inflammation.

The phrase “joint cartilage damage inhibiting effective amount” means an amount of an invention compound, compound mixture, or combination sufficient to prevent or inhibit joint cartilage damage as described above.

The term “treating” means administering to a patient an amount of one or more of the invention compounds, compound mixtures, or combinations according to an invention method, wherein the amount is sufficient to prevent initiation of, or inhibit the progress, prevent further progress, or reverse progression, in part or in whole, of any one or more of the pathological hallmarks or symptoms of any one of the diseases and disorders being prevented or treated that is appreciated, suspected, or expected, including, but not limited to, the pathological hallmark of joint cartilage damage or the symptoms of joint pain and joint inflammation.

The phrase “treating osteoarthritis” means administering to a patient an amount of one or more of the invention compounds, compound mixtures, or combinations according to an invention method, wherein the amount is sufficient to prevent initiation of, or inhibit the progress, prevent further progress, or reverse progression, in part or in whole, of a disease pathology or any one or more symptoms of osteoarthritis that is appreciated, suspected, or expected, including, but not limited to, the symptoms of joint cartilage damage, joint pain, or joint inflammation.

The phrase “osteoarthritis treating effective amount” means an amount of an invention compound, compound mixture, or combination sufficient to prevent or inhibit osteoarthritis as described above.

The phrase “treating rheumatoid arthritis” means administering to a patient an amount of one or more of the invention compounds, compound mixtures, or combinations according to an invention method, wherein the amount is sufficient to prevent initiation of, or inhibit the progress, prevent further progress, or reverse progression, in part or in whole, of a disease pathology or any one or more symptoms of rheumatoid arthritis that is appreciated, suspected, or expected, including, but not limited to, the symptoms of joint pain or joint inflammation.

The phrase “rheumatoid arthritis treating effective amount” means an amount of an invention compound, compound mixture, or combination sufficient to prevent or inhibit rheumatoid arthritis as described above.

The phrase “joint pain alleviating” means administering prophylactically to an asymptomatic patient or administering to a patient experiencing joint pain, an amount of one or more of the invention compounds, compound mixtures, or combinations according to an invention method, wherein the amount is sufficient to suppress, reduce, prevent, or otherwise inhibit joint pain symptoms in a patient, including, but not limited to, the suppression, reduction, prevention, or inhibition of joint pain symptoms due to joint cartilage damage, joint inflammation, and joint pain associated with autoimmune disorders.

The phrase “joint pain alleviating effective amount” means an amount of an invention compound, compound mixture, or combination sufficient to alleviate joint pain as described above.

The term “nontoxic” when used alone means the efficacious dose is 10 times or greater than the dose at which a toxic effect is observed in 10% or more of a patient population.

It should be appreciated that an invention compound or pharmaceutical composition may be administered in an amount that is “sufficiently nontoxic.” A sufficiently nontoxic amount may be an efficacious dose which may potentially produce toxic symptoms in certain patients at certain doses, but because of the pernicious nature of the disease being treated or the idiosyncratic nature of the appearance of the toxic symptoms in a patient population, and the risk/benefit value to the patient or patient population of the invention compound being used, it is acceptable to patients, medical or veterinary practitioners, and drug regulatory authorities to use such a sufficiently nontoxic dose. Under certain circumstances, a sufficiently nontoxic dose may be an efficacious dose at which more than 10% of a patient population experience one or more toxic symptoms but wherein the disease being treated is a life-threatening disease such as cancer, including breast cancer, and there are no better treatment options. Alternatively, a sufficiently nontoxic dose may be a generally nontoxic efficacious dose at which a certain majority of patients being treated do not experience drug-related toxicity, although a small percentage of the patient population may be susceptible to an idiosyncratic toxic effect at the dose.

It should be appreciated that preventing initiation of a disease pathology or inhibiting the progress, preventing further progress, or reversing progression, in part or in whole, of a pathological hallmark of a disease or disorder being prevented or treated means having a disease-modifying effect with, or without, having an effect on symptoms such as pain or inflammation, if present. For example, a disease modifying effect of treating joint cartilage damage may be effected by administering an invention compound to a patient in need thereof without having an effect on joint pain or joint inflammation, if present.

The phrases “therapeutically effective amount” and “effective amount” are synonymous and mean an amount of a invention compound, compound mixture, or combination that is sufficient to prevent the initiation of, or to inhibit the progress, prevent further progress, or reverse progression, in part or in whole, of a disease pathology or any one or more symptoms of a disease or disorder that is appreciated or suspected or expected in the particular patient being treated.

In determining what constitutes a therapeutically effective amount of an invention compound, or a pharmaceutically acceptable salt thereof, or a combination comprising an invention compound or compound mixture with another drug such as those described above, for treating or inhibiting according to an invention method, a number of factors will generally be considered by the medical practitioner or veterinarian in view of the experience of the medical practitioner or veterinarian, published clinical studies, the subject's (ie, mammal's) age, sex, weight and general condition, as well as the type and extent of the disease, disorder or condition being treated, and the use of other medications, if any, by the subject. Such amounts will generally be from about 0.1 mg/kg to about 300 mg/kg of subject body weight. Typical doses will be from about 1 to about 5000 mg/day for an adult subject of normal weight. In a clinical setting, regulatory agencies such as, for example, the FDA in the United States may require a particular therapeutically effective amount.

A therapeutically effective amount of an administered dose may fall within the ranges or amounts recited above, or may vary outside, i.e., either below or above, those ranges depending upon the requirements of the individual subject, the severity of the condition being treated, and the particular therapeutic formulation being employed. Determination of a proper dose for a particular situation and patient is within the ordinary skill of the medical or veterinary artisan. Generally, treatment may be initiated using smaller dosages of an invention compound, compound mixture, or combination that are less than optimum dosage for a particular patient. Thereafter, the dosage can be increased by small increments until the optimum effect under the circumstance is reached. For convenience, the total daily dosage may be divided and administered in portions during the day, if desired.

The invention methods may be conducted by administering an invention compound or an invention combination, either alone or formulated in a composition suitable for pharmaceutical administration. The invention pharmaceutical compositions may be produced by formulating the invention compound, compound mixture, or combination in dosage unit form with a pharmaceutical carrier. Some examples of dosage unit forms are tablets, capsules, pills, powders, aqueous and nonaqueous oral solutions and suspensions, and parenteral solutions packaged in containers containing either one or some larger number of dosage units and capable of being subdivided into individual doses.

Some examples of suitable pharmaceutical carriers, including pharmaceutical diluents, are gelatin capsules; sugars such as lactose and sucrose; starches such as corn starch and potato starch; cellulose derivatives such as sodium carboxymethyl cellulose, ethyl cellulose, methyl cellulose, and cellulose acetate phthalate; gelatin; talc; stearic acid; magnesium stearate; vegetable oils such as peanut oil, cottonseed oil, sesame oil, olive oil, corn oil, and oil of theobroma; propylene glycol, glycerin; sorbitol; polyethylene glycol; water; agar; alginic acid; isotonic saline, and phosphate buffer solutions; as well as other compatible substances normally used in pharmaceutical formulations.

The compositions to be employed in the invention can also contain other components such as coloring agents, flavoring agents, and/or preservatives. These materials, if present, are usually used in relatively small amounts. The compositions can, if desired, also contain other therapeutic agents commonly employed to treat a disease of the present invention methods. Further, the compositions can, if desired, also contain other therapeutic agents as described above. The other therapeutic agents may be used in an invention combination to treat a disease that is the same as, or different from, a disease of a present invention method. The other therapeutic agents may be used for disease modifying therapy or to treat secondary symptoms such as, for example, inflammation or pain. For example, the compositions may contain aspirin, naproxen, or similar anti-inflammatory analgesic agents.

The percentage of the active ingredients in the foregoing compositions can be varied within wide limits, but for practical purposes it is preferably present in a concentration of at least 10% in a solid composition and at least 2% in a primary liquid composition. The most satisfactory compositions are those in which a much higher proportion of the active ingredient is present, for example, up to about 95%.

Preferred routes of administration of an invention compound or invention combination, according to the invention methods are oral or parenteral. For example, a useful intravenous dosage is between 5 and 50 mg, and a useful oral dosage is between 20 and 800 mg. The dosage is within the dosing range used in treatment of diseases according to the invention methods, or as would be determined by a physician according to the needs of the patient as described above.

An invention compound or combination may be administered in any form. Preferably, administration is in unit dosage form.

The advantages of the instant invention compounds include the relatively nontoxic nature of the [c]-fused bicyclic proline derivatives, their ease of preparation, the fact that the invention compounds are well-tolerated, and the ease of IV and oral administration of the drugs.

Another important advantage is that the invention compounds provide much needed disease modifying activity for osteoarthritis and other diseases and disorders exhibiting joint cartilage damage by virtue of their ability to prevent and inhibit the joint cartilage damage. Aspirin and conventional nonsteroidal anti-inflammatory drugs (NSAIDs) such as ibuprofen, diclofenac, and naproxen are the primary agents used to treat joint pain resulting from joint cartilage damage, including OA-related joint pain. These agents inhibit prostaglandin release by blocking cyclooxygenase-mediated conversion of cell membrane lipids from arachidonic acid. However, the therapeutic use of conventional NSAIDs is limited due to drug associated side effects, including life threatening ulceration and renal toxicity. Further, typically each of these drugs treat secondary conditions associated with joint cartilage damage or osteoarthritis such as joint pain, but do not prevent or treat the primary condition, which is damage to the cartilage (Chapter 18: The Pharmacologic Treatment of Osteoarthritis by Simon, L. S. and Strand, V., supra, p. 371).

Another important advantage of the instant invention is that certain invention compounds do not displace (i.e., IC₅₀≧10 micromolar), or only weakly displace (i.e., 1 micromolar≦IC₅₀<10 micromolar), gabapentin from an alpha-2-delta receptor subtype 1 or 2, and thus are not expected to adversely interact with pharmaceuticals such as gabapentin that provide therapeutic benefit in patients and bind to an alpha-2-delta receptor. While all of the invention compounds have valuable therapeutic advantages for use according to the invention methods, the subset of invention compounds that do not displace, or weakly displace, gabapentin from an alpha-2-delta receptor have the additional advantage of not being contraindicated in patients being treated with drugs such as gabapentin that bind to an alpha-2-delta receptor.

It should be appreciated that for the purposes of the present invention, determination of the ability of an invention compound to displace gabapentin from an alpha-2-delta receptor is carried out with pig alpha-2-delta receptor 1 according to Biological Method 5 below.

Another important advantage of the instant invention is that certain invention compounds do not bind to the leucine transport system (“LTS”), which is a system that transports amino acids such as leucine across the blood-brain barrier. Compounds that do not cross the blood-brain barrier will not produce brain-mediated central nervous system side effects in vivo.

It should be appreciated that for the purposes of the present invention, determination of the ability of an invention compound to bind to the LTS is carried out with CHO K1 cells according to Biological Method 7 below.

Another important advantage of the instant invention is that certain invention compounds exhibit preferred mean drug half-lives in plasma when administered perorally or by intravenous infusion to three patients. In three human patients, a preferred mean plasma half-life for quaque die (“QD,” meaning once daily) peroral dosing is from about 12 hours to about 24 hours. In three rats, a preferred mean plasma half-life for QD peroral dosing is from 4.0 hours to about 10 hours. More preferred in three rats is from 6.0 hours to about 10 hours.

It should be appreciated that for the purposes of the present invention, the determination of a mean drug half-life in plasma in three rats is carried out according to the peroral infusion administration methods described below in Biological Method 8.

It should be further appreciated that an invention compound that has a mean plasma half-life in hours following peroral or intravenous infusion administration to three patients that is below a preferred range for QD administration may optionally be administered bis in die (“BID,” meaning twice daily), ter in die (“TID,” meaning three times a day), or quater in die (“QID,” meaning four times a day), in order to obtain plasma levels of the compound that are optimal for treatment of a patient. Conversely, an invention compound that has a mean plasma half-life in hours following peroral or intravenous infusion administration to three patients that is above a preferred range for QD administration may optionally be administered once every two days or once per week, for example, in order to obtain plasma levels of the compound that are optimal for treatment of a patient.

Another important advantage of the instant invention is that certain invention compounds exhibit preferred mean bioavailability in plasma of from about 50% to 100% when administered perorally to three patients.

It should be appreciated that for the purposes of the present invention, the determination of mean bioavailability is carried out in three rats according to the peroral method described below in Biological Method 8.

Another advantage is that the instant invention may, if desired, allow the amount of an anti-inflammatory agent and/or pain alleviating agent used in the treatment of patients suffering from joint cartilage damage and other symptoms such as joint inflammation and/or joint pain to be reduced or even eliminated. It is known that anti-inflammatory and analgesic agents may produce undesirable side effects such as gastro-intestinal bleeding and ulceration. These side effects may be avoided, reduced or eliminated by using the instant invention to inhibit joint cartilage damage.

When administered to a patient according to an invention method, an invention compound may be converted in vivo by biological conversion of the administered stereoisomer of the invention compound to another stereoisomer of the invention compound. One possible biological conversion of stereoisomers in vivo would be epimerization of the hydrogen atom at the C-1 carbon atom of Formula I wherein R² is H (i.e., epimerization of the hydrogen atom alpha to the Z substituent). Epimerization could result in compound mixtures, or mixtures of 2 or more stereoisomers of the invention compounds. Compound mixtures may also be prepared by conventional synthetic organic chemistry methods and administered as such to a patient according to an invention method. Compound mixtures are included in the scope of the present invention.

The invention compound and compound mixtures may be prepared by conventional synthetic organic chemistry, which may be carried out by one of ordinary skill in the art of organic chemistry by adapting various synthetic procedures that are well-known in the art of organic chemistry. The synthetic organic chemistry preparation of an invention compound or compound mixture may proceed through a number of intermediates, any of which intermediates may be prepared by one of ordinary skill in the art of organic chemistry by adapting various synthetic procedures that are well-known in the art of organic chemistry. These synthetic procedures may be found in the literature in, for example, Reagents for Organic Synthesis, by Fieser and Fieser, John Wiley & Sons, Inc, New York, 2000; Comprehensive Organic Transformations, by Richard C. Larock, VCH Publishers, Inc, New York, 1989; the series Compendium of Organic Synthetic Methods, 1989, by Wiley-Interscience; the text Advanced Organic Chemistry, 4^{th} edition, by Jerry March, Wiley-Interscience, New York, 1992; or the Handbook of Heterocyclic Chemistry by Alan R. Katrisky, Pergamon Press Ltd, London, 1985, to name a few. Alternatively, a skilled artisan may find methods useful for preparing the intermediates in the chemical literature by searching widely available databases such as, for example, those available from the Chemical Abstracts Service, Columbus, Ohio, or MDL Information Systems GmbH (formerly Beilstein Information Systems GmbH), Frankfurt, Germany.

Preparations of invention compounds may use starting materials, reagents, solvents, and catalysts that may be purchased from commercial sources or they may be readily prepared by adapting procedures in the references or resources cited above. Commercial sources of starting materials, reagents, solvents, and catalysts useful in preparing invention compounds include, for example, The Aldrich Chemical Company, and other subsidiaries of Sigma-Aldrich Corporation, St. Louis, Mo., BACHEM, BACHEM A. G., Switzerland, or Lancaster Synthesis Ltd, United Kingdom.

Syntheses of some invention compounds may utilize starting materials, intermediates, or reaction products that contain a reactive functional group. During chemical reactions, a reactive functional group may be protected using protecting groups that render the reactive group substantially inert to the reaction conditions employed. A protecting group is introduced onto a starting material prior to carrying out the reaction step for which a protecting group is needed. Once the protecting group is no longer needed, the protecting group can be removed. It is well within the ordinary skill in the art to introduce protecting groups during a synthesis of an invention compound, and then later remove them. Procedures for introducing and removing protecting groups are known and referenced such as, for example, in Protective Groups in Organic Synthesis, 2^{nd} ed., Greene T. W. and Wuts P. G., John Wiley & Sons, New York: New York, 1991, which is hereby incorporated by reference. Thus, for example, protecting groups such as the following may be utilized to protect amino, hydroxyl, and other groups: carboxylic acyl groups such as, for example, formyl, acetyl, and trifluoroacetyl; alkoxycarbonyl groups such as, for example, ethoxycarbonyl, tert-butoxycarbonyl (BOC), β,β,β-trichloroethoxycarbonyl (TCEC), and β-iodoethoxycarbonyl; aralkyloxycarbonyl groups such as, for example, benzyloxycarbonyl (CBZ), para-methoxybenzyloxycarbonyl, and 9-fluorenylmethyloxycarbonyl (FMOC); trialkylsilyl groups such as, for example, trimethylsilyl (TMS) and tert-butyldimethylsilyl (TBDMS); and other groups such as, for example, triphenylmethyl (trityl), tetrahydropyranyl, vinyloxycarbonyl, ortho-nitrophenylsulfenyl, diphenylphosphinyl, para-toluenesulfonyl (Ts), mesyl, trifluoromethanesulfonyl, and benzyl. Examples of procedures for removal of protecting groups include hydrogenolysis of CBZ groups using, for example, hydrogen gas at 50 psi in the presence of a hydrogenation catalyst such as 10% palladium on carbon, acidolysis of BOC groups using, for example, hydrogen chloride in dichloromethane, trifluoroacetic acid (TFA) in dichloromethane, and the like, reaction of silyl groups with fluoride ions, and reductive cleavage of TCEC groups with zinc metal.

Preparations of starting materials useful in the preparation of an invention compound are incorporated by reference to the patents or patent application publications described above and below.

It should be appreciated that unless otherwise noted below, reagents and solvents were used as received from commercial suppliers. Proton and carbon nuclear magnetic resonance were obtained on a Bruker AC 300, ACE 300 MHz or a Bruker AV-300 spectrometer at 300 MHz for proton and 75 MHz for carbon. Tetramethylsilane was used as an internal standard for proton spectra and the solvent peak was used as the reference peak for carbon spectra. IR spectra were obtained by attenuated total reflectance (ATR) on a Nicolet 470 spectrometer. Mass spectra were obtained on either a Thermo Finnigan LCQ Duo LCMS ion trap electrospray ionization (ESI), or a Perkin Elmer Sciex 100 atmospheric pressure ionization (APCI) mass spectrometer. Melting points were determined by a Mettler Toledo Model 821 Differential Scanning Calorimeter (DSC), or by a Thomas-Hoover (oil bath) or Mel-Temp II (hot stage) apparatus (uncorrected). Elemental analyses were performed by Quantitative Technologies, Inc. (Whitehouse, N.J.).

It should be appreciated that the following abbreviations are used below in the description of the preparation of the invention compounds:

PLE is Pig Liver Esterase
DMF is Dimethylformamide
THF is Tetrahydrofuran
6N HCl is 6 normal hydrochloric acid
BOC is tert-butyloxycarbonyl
CBZ is benzyloxycarbonyl
Ra is Raney
Et₃N is triethylamine
6M is 6 molar
NCS is N-chlorosuccinimide
DBU is 1,8-diazabicyclo[5.4.0]undec-7-ene
TMS-CN is Trimethylsilylcyanide
EDC is 1-(3-dimethylaminopropyl)-3-ethylcarbodiimide hydrochloride
HOBt is 1-hydroxybenzotriazole
CH₃SO₂Cl is methanesulfonyl chloride
DMAP is 4-dimethylaminopyridine
[3+2] is a three plus two cyclization
LDA is Lithium diisopropylamide
CAS is Chemical Abstracts Service
n-BuLi is Normal-butyl lithium
KHMDS is potassium hexamethyldisilazide
mCPBA is meta-chloroperbenzoic acid
DMSO is dimethylsulfoxide
¹H-NMR is proton nuclear magnetic resonance
Pd₂(dba)₃ is tris(1,5-diphenyl-1,4-pentadien-3-one)dipalladium(0), also known as tris(dibenzylidene-acetone)dipalladium(0)

Preparation of (1S,3aS,7aR)- and (1R,3aR,7aS)-1,3,3a,4,5,6,7,7a-octahydroisoindole-1-carboxylic acid by conventional means is illustrated below in Preparation Scheme A.

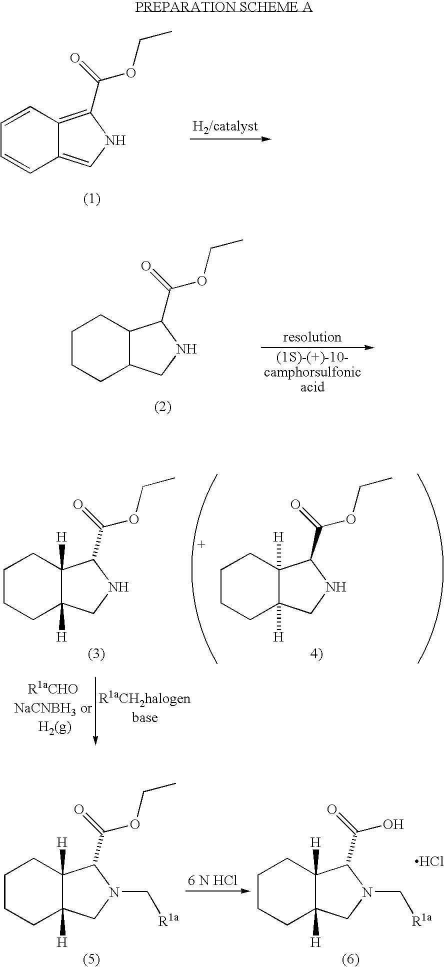

R^{1a}═H, alkyl, phenyl, heteroaryl, etc.

It should be appreciated that a compound of formula R^{1a}CH₂-LG, wherein LG is acetoxy, trifluoroacetoxy, methanesulfonyloxy, trifluoromethanesulfonyl, para-toluenesulfonyloxy, and the like may be used in place of R^{1a}CH₂halogen.

It should be appreciated that Preparation Scheme A may be adapted to prepare other stereoisomers of 1,3,3a,4,5,6,7,7a-octahydroisoindole-1-carboxylic acid.

Certain synthetic preparations of a compound of Formula I which is a 2-substituted 1,3,3a,4,5,6,7,7a-octahydroisoindole-1-carboxylic acid involve a methylation of the corresponding 1,3,3a,4,5,6,7,7a-octahydroisoindole-1-carboxylic acid, or a protected form thereof such as 1,3,3a,4,5,6,7,7a-octahydroisoindole-1-carboxylic acid methyl ester. One such methylation reaction is adapted for the preparation of the invention compounds as shown below in Scheme A.

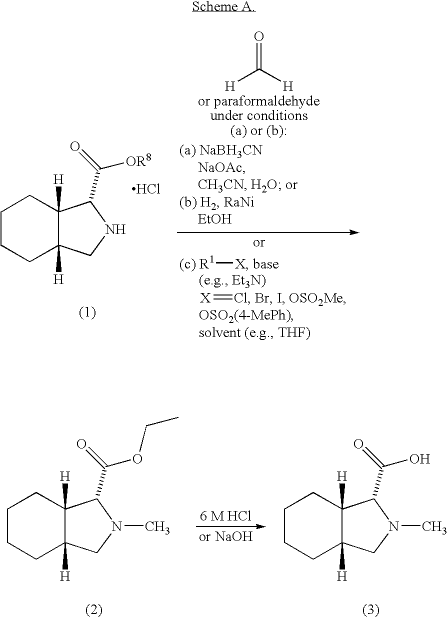

Compounds of Formula I wherein R² is not H may be prepared by conventional means as illustrated below in Schemes B1 and B2.

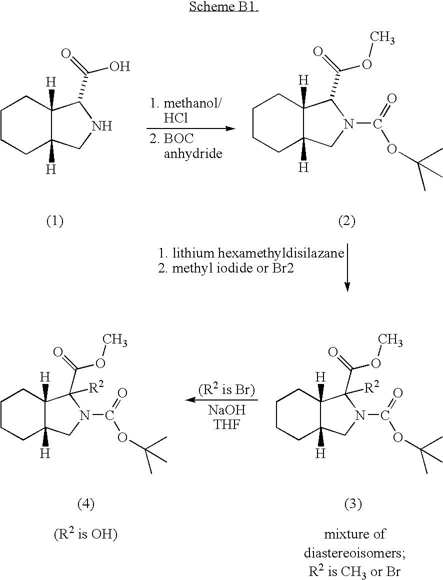

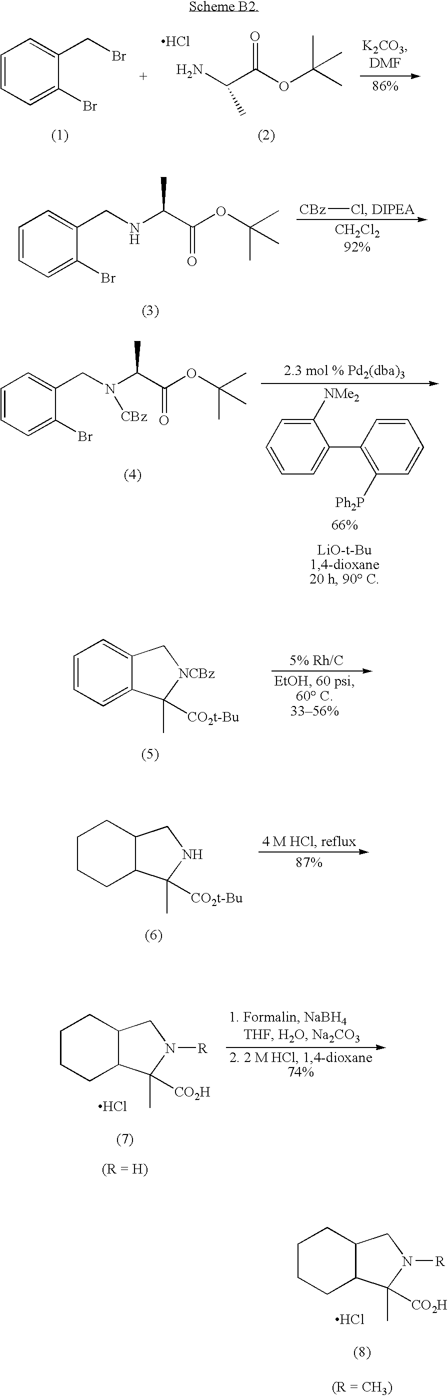

Adapting the methods illustrated above in Scheme B2, the compounds of Compound Examples B 1 to B5 were prepared.

### COMPOUND EXAMPLE B1

### Synthesis of 1,2-dimethyl-octahydro-isoindole-1-carboxylic Acid Hydrochloride

To a suspension of the compound of Compound Example B2 (1.02 g, 4.65 mmol) in water (4 mL), was added sodium carbonate (0.49 g, 4.65 mmol) followed by formalin (2.0 mL, 37% solution, 23.2 mmol). After stirring for 10 minutes, the mixture was added dropwise to a stirred solution of sodium borohydride (0.44 g, 11.6 mmol) in tetrahydrofuran (20 mL). The reaction mixture was stirred for 12 hours at room temperature and then concentrated to dryness. The crude residue was dissolved in methanol, reduced in volume to approximately 5 mL, and applied directly to a silica gel column (eluant dichloromethane-/methanol/ammonium hydroxide 90:10:2) to afford the desired product (0.8 g, 74%) as a white solid. This white solid was stirred in 2 M HCl (20 mL, ether) for 3 hours and the product was collected by filtration and dried (0.9 g): mp: 67-68° C.; ¹H NMR (300 MHz, CD₃OD) δ 1.20-1.38 (m, 3H), 1.50-1.52 (m, 1H), 1.55 (s, 3H), 1.64-1.70 (m, 4H), 2.15-2.17 (m, 1H), 2.79 (s, 3H), 2.81-2.83 (m, 1H), 3.18 (t, J=9.9 Hz, 1H), 3.65 (t, J=9.9 Hz, 1H); MS (ESI) m/z 198 [M+H]⁺.

Anal. Calcd. for C₁₁H₁₉NO₂—HCl-0.3H₂O: C, 55.25; H, 8.68; N, 5.86. Found: C, 55.10; H, 8.54; N, 5.80.

### COMPOUND EXAMPLE B2

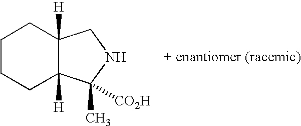

### Synthesis of a Racemic Mixture of (1S,3aS,7aR)-1-methyl-octahydro-isoindole-1-carboxylic Acid Hydrochloride and (1R,3aR,7aS)-1-methyl-octahydro-isoindole-1-carboxylic Acid Hydrochloride

Step (1): Preparation of 1-methyl-octahydro-isoindole-1-carboxylic Acid tert-butyl Ester

To a Parr bottle containing 5% Rh/C (0.45 g) was added methanol (50 mL) under an atmosphere of nitrogen. The mixture was shaken with hydrogen (40 psi) for 20 minutes to pre-reduce the catalyst. A solution of 1-methyl-1,3-dihydro-isoindole-1,2-dicarboxylic acid 2-benzyl ester 1-tert-butyl ester (Buchwald, S. L. et al. J. Org. Chem. 2002, 67, 465-475, 1.3 g, 3.05 mmol) in methanol (100 mL) was added to the pre-reduced catalyst and the reaction mixture shaken at 60° C. for 24 hours under hydrogen (60 psi). The mixture was filtered through a pad of celite, concentrated and purified by silica gel chromatography using hexanes/ethyl acetate (90:10) as eluant to afford the title compound (0.4 g, 56%) as a colorless oil: ¹H NMR (300 MHz, CD₃OD) δ 1.09-1.26 (m, 4H), 1.34 (s, 3H), 1.47 (s, 9H), 1.51-1.68 (m, 4H), 1.84-1.91 (m, 1H), 2.62-2.64 (m, 1H), 2.87 (dd, J=10.2, 8.4 Hz, 1H), 3.05 (t, J=10.7 Hz, 1H); ¹³C NMR (75 MHz, CD₃OD) δ 21.8, 24.7, 25.7, 26.4, 28.3, 37.7, 47.7, 48.4, 71.5, 82.8, 175.1; MS (ESI) m/z 240 [M+H]⁺.

Step (2): Synthesis of a Racemic Mixture of (1S,3aS,7aR)-1-methyl-octahydro-isoindole-1-carboxylic Acid Hydrochloride and (1R,3aR,7aS)-1-methyl-octahydro-isoindole-1-carboxylic Acid Hydrochloride

A mixture of the product of Step (1) (1.65 g, 6.89 mmol) and 20% aqueous HCl (60 m-L) was heated to reflux for 4 hours. The reaction mixture was allowed to cool to room temperature, and the solvent was removed in vacuo. The white solid was stirred in ether (20 mL) for 3 hours and the product was collected by filtration and dried (1.3 g, 87%): mp: 237-240° C.; ¹H NMR (300 MHz, CD₃OD) δ 1.83-1.31 (m, 3H), 1.54-1.60 (m, 1H), 1.68-1.74 (m, 7H), 2.21 (dt, J=17.4, 11.6, 5.6 Hz, 1H), 2.93-2.94 (m, 1H), 3.31-3.32 (m, 1H), 3.45 (dd, J=9.3, 11.8 Hz, 1H); ¹³C NMR (75 MHz, CD₃OD) δ 21.0, 22.3, 24.2, 24.8, 24.9, 36.2, 46.7, 46.8, 74.6, 172.7; MS (APCI) m/z 184 [M+H]⁺. Anal. Calcd. for C₁₀H₁₇NO₂—HCl-0.1H₂O: C, 54.22; H, 8.28; N, 6.32; Cl, 16.00. Found: C, 53.87; H, 8.27; N, 6.30; Cl, 16.08; relative stereochemistry was determined by Nuclear Overhauser Effect nuclear magnetic resonance experiments.

### COMPOUND EXAMPLE B3

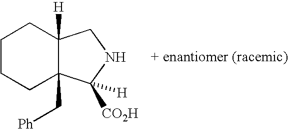

### Synthesis of a Racemic Mixture of (1R,3aS,7aS)-7a-benzyl-octahydro-isoindole-1-carboxylic Acid Hydrochloride and (1S,3aR,7aR)-7a-benzyl-octahydro-isoindole-1-carboxylic Acid Hydrochloride

Following a similar procedure to that described below for Compound Example B4, Steps (1) to (5), the title compound was obtained as off-white needles after recrystallization from water: ¹H NMR (300 MHz, D₂O) δ 7.24-7.42 (m, 5H), 4.46 (s, 1H), 3.88 (dd, J=12.1, 5.8 Hz, 1H), 3.12 (d, J=12.1 Hz, 1H), 2.76 (d, J=13.6 Hz, 1H), 2.45 (d J=13.6 Hz, 1H), 2.20 (quintet, J=5.9 Hz, 1H), 2.00 (d, J=14.2 Hz, 1H), 1.55-1.67 (m, 4H), 1.41 (q, J=12.3 Hz, 1H), 1.17 (q, J=12.4 Hz, 1H), 0.87 (q, J=11.8 Hz, 1H); ¹³C NMR (75 MHz, D₂O) δ 170.5, 137.2, 132.4, 129.8, 128.5, 64.3, 51.1, 40.9, 40.5, 29.4, 29.1, 25.7, 22.1; MS (ESI) m/z 260 [C₁₆H₂₁NO₂+H]⁺, 282 [M+Na]⁺, 519 [2M+H]⁺; HPLC Analysis (Method B: Phenomenex Synergi Hydro-RP Column, Detector @ 254 nm) 97.7%, t_{R}=16.0 min. Anal. Calcd for C₁₆H₂₁NO₂.0.1NH₃.0.75H₂O: C, 69.99; H, 8.37; N, 5.61. Found: C, 69.67; H, 8.34; N, 5.46; relative stereochemistry was determined by Nuclear Overhauser Effect nuclear magnetic resonance experiments.

### COMPOUND EXAMPLE B4

### Synthesis of 7a-methyl-octahydro-isoindole-1-carboxylic Acid

Step (1): Preparation of 1-oxo-octahydro-isoindole-2-carboxylic Acid tert-butyl Ester

A solution of octahydro-isoindol-1-one (CAS Registry Number 2555-11-5, 15.0 g, 108 mmol), Boc₂O (35.3 g, 162 mmol), Et₃N (30.0 mL, 216 mmol), and DMAP (2.60 g, 21.6 mmol) in THF (600 mL) was stirred at room temperature for 15.5 h, then the reaction mixture was concentrated. The residue was diluted with CH₂Cl₂ (500 mL), washed with 0.05 M HCl and brine, dried over sodium sulfate, filtered, and concentrated. The residue was purified by column chromatography (CH₂Cl₂, then 1-2% methanol in CH₂Cl₂) to provide title compound as a yellow oil (21.0 g, 81%), which solidified on standing: ¹H NMR (300 MHz, CDCl₃) δ 3.62 (dd, J=6.0, 10.7 Hz, 1H), 3.42 (dd, J=1.9, 10.7 Hz, 1H), 2.59 (m, 2H), 2.28 (m, 1H), 2.10 (m, 1H), 1.74 (m, 1H), 1.46-1.67 (m, 3H), 1.54 (s, 9H), 1.18-1.32 (m, 3H); MS (APCI) m/z 184 [C₁₃H₂₁NO₃+H−CH₂═C(CH₃)₂]+, 140 [M−Boc+H]⁺.

Step (2): Preparation of 3a-methyl-3-oxo-octahydro-isoindole-2-carboxylic Acid tert-butyl Ester

To a stirred solution of lithium diisopropylamide (LDA; 1.8 M solution in heptane/THF/ethylbenzene, 46.5 mL) and THF (500 mL) at −78° C. was added dropwise a solution of 1-oxo-octahydro-isoindole-2-carboxylic acid tert-butyl ester (10.0 g, 41.8 mmol) in THF (100 mL). After stirring the mixture for 1 hour at −78° C., iodomethane (13.0 mL, 209 mmol) was added dropwise. The reaction mixture was stirred at −78° C. for 3.5 hours, and water (10 mL) was added. The organic solvent was evaporated in vacuo and the aqueous layer was extracted with CH₂Cl₂ (3×150 mL). The combined organic layers were washed with 0.05 M HCl and brine, dried over sodium sulfate, filtered, and concentrated. The residue was purified by column chromatography (CH₂Cl₂, then 1-2% methanol in CH₂Cl₂) to provide the title compound (5.01 g, 47%) as a yellow oil: ¹H NMR (300 MHz, CDCl₃) δ 3.70 (dd, J=10.8, 6.9 Hz, 1H), 3.40 (dd, J=10.8, 6.0 Hz, 1H), 1.96 (quintet, J=6.3 Hz, 1H), 1.68-1.81 (m, 2H), 1.54 (s, 9H), 1.25-1.52 (m, 6H), 1.18 (s, 3H); MS (ESI) m/z 529 [2M+H]⁺.

Step (3): Preparation of 3a-methyl-3-hydroxy-octahydro-isoindole-2-carboxylic Acid tert-butyl Ester

To a stirred solution of the product of Step (2) (5.00 g, 19.8 mmol) in THF (120 mL) at −78° C. was added dropwise lithium triethylborohydride (1 M in THF, 23.7 mL, 23.7 mmol). The reaction mixture was stirred at −78° C. for 2 hours, quenched with saturated aqueous sodium bicarbonate (60 mL), then warmed to 0° C. Hydrogen peroxide (35%, 6 mL) was added and the mixture was stirred at 0° C. for 30 minutes. The organic solvent was evaporated in vacuo and the aqueous layer was extracted with CH₂Cl₂ (3×120 mL). The combined organic layers were dried over sodium sulfate, filtered, and concentrated, to provide 3a-methyl-3-hydroxy-octahydro-isoindole-2-carboxylic acid tert-butyl ester (3.8 g, 75%) as a pale yellow solid. This was used in the next step without further purification: ¹H NMR (300 MHz, CDCl₃) δ 4.96 & 4.22 (two d, J=1.6 Hz, 1H), 3.25-3.45 (m, 1H), 1.76-1.85 (m, 1H), 1.25-1.64 (m, 8H), 1.49 & 1.47 (two s, 9H), 1.09 & 1.07 (two s, 3H); MS (ESI) m/z 238 [C₁₄H₂₅NO₃—H₂O+H]⁺.

Step (4): 3a-methyl-3-cyano-octahydro-isoindole-2-carboxylic Acid tert-butyl Ester

To a stirred solution of the compound of Step (3) (3.8 g, 15 mmol) and trimethylsilyl cyanide (5.3 μL, 40 mmol) in CH₂Cl₂ (300 mL) at −78° C. was added dropwise boron trifluoride diethyl etherate (5.5 mL, 44 mmol). The reaction mixture was stirred at −78° C. for 4 hours, quenched with saturated aqueous sodium bicarbonate (30 mL), and extracted with CH₂Cl₂ (3×100 mL). The combined organic layers were dried over sodium sulfate, filtered, and concentrated. The residue was purified by column chromatography (CH₂Cl₂, then 1% methanol in CH₂Cl₂) to provide a diastereoisomeric mixture of 3a-methyl-3-cyano-octahydro-isoindole-2-carboxylic acid tert-butyl ester (2.7 g, 52%) as a yellow syrup: ¹H NMR (300 MHz, CDCl₃) δ 4.22 & 4.10 (two s, 1H), 3.29-3.56 (m, 2H), 1.13-2.19 (m, 1H), 1.26-1.73 (m, 8H), 1.52 & 1.49 (two s, 9H), 1.31 & 1.30 (two s, 3H); MS (ESI) m/z 265 [C₁₄H₂₅NO₃+H]⁺, 551 [2M+Na]⁺.

Step (5): Synthesis of 7a-methyl-octahydro-isoindole-1-carboxylic Acid

The compound of Step (4) was dissolved in concentrated HCl (200 mL) and heated at reflux for 16.5 hours. The reaction mixture was cooled to room temperature, washed with diethyl ether (3×50 mL), and concentrated. The residual solid was purified by column chromatography on silica gel using methylene chloride/methanol and concentrated ammonium hydroxide in a gradient of 14:4:1 to 6:3:1 to provide the title compound (0.38 g, 17%) as a white powder: ¹H NMR (300 MHz, D₂O) δ 4.32 (s, 1H), 3.65 (dd, J=11.9, 6.5 Hz, 1H), 3.15 (dd, J=11.9, 3.0 Hz, 1H), 1.96-2.17 (m, 1H), 1.93-1.98 (m, 1H), 1.12-1.75 (m, 7H), 1.04 (s, 3H); ¹³C NMR (75 MHz, D₂O) δ 173.3, 65.4, 49.2, 44.4, 43.3, 32.7, 26.5, 23.5, 22.7, 21.1; IR (ATR) 3087, 2924, 2853, 1589, 1452, 1340, 1304, 1277, 894, 748, 594, 455 cm⁻¹; MS (ESI) m/z 184 [C₁₀H₁₇NO₂+H]⁺, 206 [M+Na]⁺.

Anal. Calcd for C₁₀H₁₇NO₂.0.2NH₃.0.5H₂O: C, 61.39; H, 9.58; N, 8.59. Found: C, 61.65; H, 9.32; N, 8.32.

### COMPOUND EXAMPLE B5

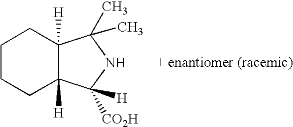

### Synthesis of a Racemic Mixture of (1S,3aR,7aR)-3,3-dimethyloctahydro-isoindole-1-carboxylic Acid Hydrochloride and (1R,3aS,7aS)-3,3-dimethyloctahydro-isoindole-1-carboxylic Acid Hydrochloride

Step (1): Preparation of 2-(1-methyl-1-nitroethyl)cyclohexanecarboxylic Acid Methyl Ester

A stirred mixture of methyl-1-cyclohexene-1-carboxylate (24.8 g, 176.9 mmol), 2-nitropropane (32 μL, 357 mol), and 1 M tetrabutylammonium fluoride (“TBAF”) in THF (265 mL, 265 mmol) was heated at 80° C. under nitrogen for 4 days. The reaction mixture was cooled to room temperature then diluted with EtOAc (200 mL). The mixture was washed with 2 M HCl (100 mL) and brine (200 mL), dried over Na₂SO₄, filtered, and evaporated. Purification of the residue by column chromatography on silica gel (14:1 hexanes/Et₂O) provided 2-(1-methyl-1-nitroethyl)cyclohexanecarboxylic acid methyl ester as Isomer 1 (>2.56 g, 6.3%, oil): ¹H NMR (300 MHz, CD₃OD) δ 3.66 (s, 3H), 2.76-2.84 (m, 1H), 2.24-2.31 (m, 1H), 1.86-2.03 (m, 3H), 1.61-1.76 (m, 2H), 1.55 (s, 3H), 1.48 (s, 3H), 1.24-1.40 (m, 3H); IR (ATR) 2942, 1735, 1540, 1349, 1257, 632 cm⁻¹; and Isomer 2 (15.14 g, 37.2%, oil): ¹H NMR (300 MHz, CD₃OD) δ 3.65 (s, 3H), 2.51 (dt, J=3.4, 11.7 Hz, 1H), 2.31 (dt, J=3.6, 11.4 Hz, 1H), 1.89-1.95 (m, 1H), 1.74-1.83 (m, 2H), 1.54-1.65 (m, 1H), 1.52 (s, 3H), 1.51 (2, 3H), 1.02-1.43 (m 4H); IR (ATR) 2942, 1735, 1540, 1349, 1257, 632 cm⁻¹.

Step (2): Preparation of 3,3-dimethyl-octahydro-isoindol-1-one

A solution of Isomer 2 from Step (1) (15.1 g, 66.0 mmol) in MeOH (420 mL) was hydrogenated (60 psi) over Raney nickel (4 mL) for 4 days. The reaction mixture was filtered through a bed of celite to remove the catalyst and the filtrate was evaporated under reduced pressure. The title compound (9.94 g, 90%) was obtained as a white solid after trituration with a hexanes/Et₂O mixture: mp 182-186° C.; ¹H NMR (300 MHz, DMSO-d₆) δ 7.52 (br s, 1H), 1.83-1.93 (m, 2H), 1.67-1.75 (m, 3H), 1.33-1.41 (m, 1H), 1.11-1.21 (m, 1H), 1.14 (s, 3H), 1.10 (s, 3H); IR (ATR) 2938, 2917, 2851, 2568, 1602, 1526, 1379, 1284, 895, 764, 704 cm⁻¹; MS (ESI) m/z 168 [C₁₀H₁₇NO+H]⁺, 335 [2M+H]⁺.

Step (3): Preparation of 1,1-dimethyl-3-oxo-octahydro-isoindole-2-carboxylic Acid tert-butyl Ester

To a stirred solution of the product from Step (2) (892 mg, 5.33 mmol), CH₂Cl₂ (25 mL), Et₃N (0.85 mL, 6.1 mmol) and 4-dimethylamino-pyridine (“DMAP”) (0.66 g, 5.4 mmol) was added BOC anhydride (“Boc₂O”) (3.66 g, 16.8 mmol) under nitrogen. The reaction mixture was stirred overnight then was diluted with EtOAc (75 mL). The organic layer was washed with 0.25 M HCl (46 mL, 12 mmol), water (50 mL) and brine (50 mL), dried over Na₂SO₄, filtered, and evaporated. Purification of the residue by column chromatography on silica gel (3:1 hexanes/Et₂O) provided the title compound (1.24 g, 87.3%) as a white solid: ¹H NMR (300 MHz, CDCl₃) δ 2.15-2.19 (m, 1H), 1.86-2.02 (m, 3H), 1.75-1.80 (m, 1H), 1.53 (s, 9H), 1.45 (s, 3H), 1.27-1.61 (m, 2H), 1.27 (s, 3H), 1.17-1.24 (m, 3H); IR (ATR) 2986, 2930, 2857, 1774, 1687, 1307, 1295, 1157, 1126, 943, 770 cm⁻¹; MS (ESI) m/z 557 [2M+Na]⁺.

Step (4): Synthesis of a Racemic Mixture of (1S,3aR,7aR)-3,3-dimethyloctahydro-isoindole-1-carboxylic Acid Hydrochloride and (1R,3aS,7aS)-3,3-dimethyloctahydro-isoindole-1-carboxylic Acid Hydrochloride

In a manner similar to that described above for Compound Example B4, Steps (3) to (5), 1,1-dimethyl-3-oxo-octahydro-isoindole-2-carboxylic acid tert-butyl ester was converted to 3,3-dimethyloctahydro-isoindole-1-carboxylic acid hydrochloride (408 mg, 81.8%, a white solid): mp 220-224° C.; ¹H NMR (300 MHz, CD₃OD) δ 4.33 (d, J=8.91 Hz, 1H), 2.24-3.32 (m, 1H), 2.08-2.12 (m, 1H), 1.78-1.88 (m, 3H), 1.57 (s, 3H), 1.48-1.60 (m, 1H), 1.30 (s, 3H), 1.10-1.28 (m, 4H); IR (ATR) 2930, 2859, 2742, 2494, 1724, 1547, 1377, 1202, 883, 829, 779 cm⁻¹; ¹³C NMR (75 MHz, CD₃OD) δ 170.9, 69.3, 62.5, 52.1, 45.4, 29.1, 26.6, 26.4, 26.3, 26.0, 22.2; MS (ESI) m/z 198 [C₁₁H₁₉NO₂+H]⁺. Anal. Calcd. for C₁₁H₁₉NO₂.HCl: C, 56.52; H, 8.62; N, 5.99; Cl, 15.17. Found: C, 56.09; H, 8.45; N, 5.72; Cl, 14.89; relative stereochemistry was determined by Nuclear Overhauser Effect nuclear magnetic resonance experiments.

Compounds of Formula I wherein R² is, for example, cycloalkyl, cycloalkenyl, or heterocycloalkyl may be prepared by conventional means by the method illustrated below in Scheme C.

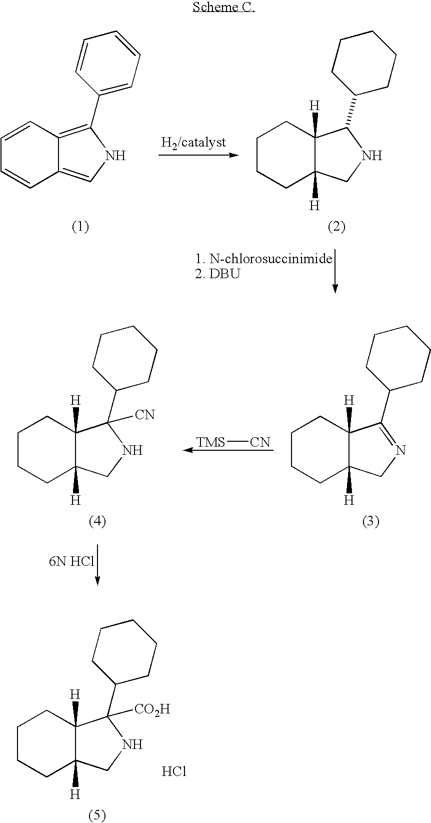

Compounds of Formula I wherein R¹ is an aryl or a heteroaryl may be prepared by conventional means by the method illustrated below in Scheme D.

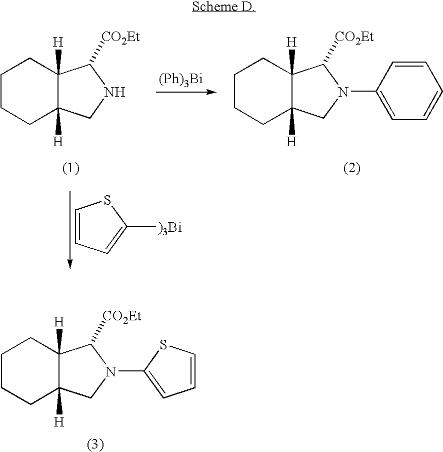

Compounds of Formula I wherein Z is C(O)N(H)R⁹ may be prepared by conventional means as illustrated below in Scheme E.

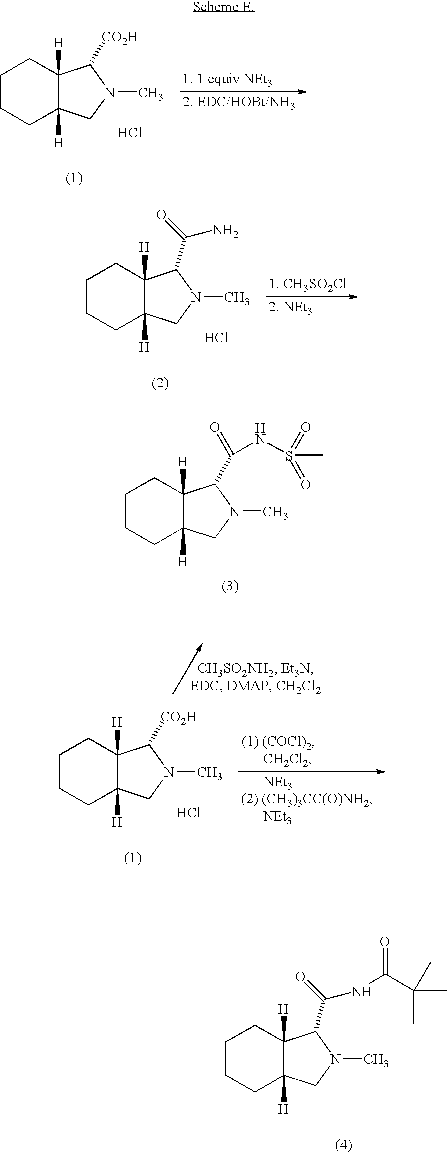

Compounds of Formula I wherein Z is Z¹ may be prepared by conventional means according to the methods illustrated below in Scheme F.

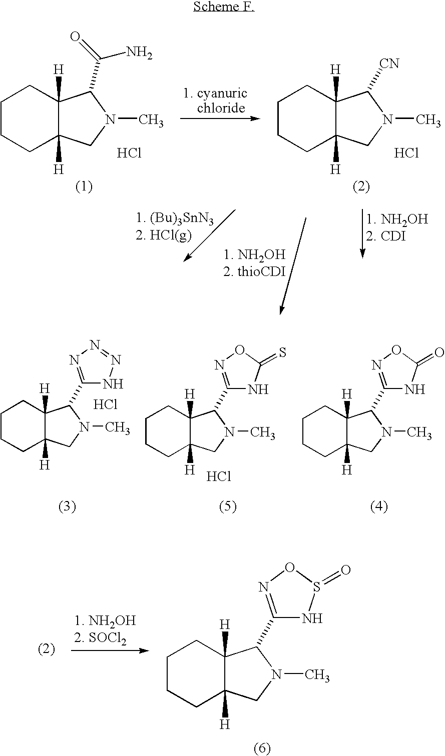

Adapting the methods illustrated above in Scheme F, the invention compounds of Compound Examples F1 and F2 were prepared as described below.

### COMPOUND EXAMPLE F1

### Synthesis of 3-(octahydro-isoindol-1-yl)-4H-[1,2,4]oxadiazol-5-one

Step (1): Preparation of benzyloxycarbonyl-amino Acetic Acid tert-butyl Ester

To a stirred solution of glycine tert-butyl ester (50.0 g, 298 mmol) and diisopropylethylamine (114 mL, 656 mmol) in methylene chloride (500 mL) at 0° C., was added benzyl chloroformate (51 mL, 360 mmol) dropwise. The reaction mixture was stirred for 3 hours, diluted with methylene chloride (1 L) and washed with water (2×500 mL), the aqueous layer was back extracted with methylene chloride (2×250 mL), and then the combined organic layers washed with brine (1×500 mL), dried over sodium sulfate, filtered and concentrated. The crude material was purified by silica gel chromatography (eluant 90:10 hexane/ethyl acetate) to afford the title compound (78.9 g, 99%) as a colorless oil: ¹H NMR (300 MHz, DMSO-d₆) δ 1.39 (s, 9H), 3.64 (d, J=7.0 Hz, 2H), 5.05 (s, 2H), 7.30-7.41 (m, 4H), 7.62 (t, J=7.5 Hz, 1H).

Step (2): Preparation of [Benzyloxycarbonyl-2-(bromo-benzyl)-amino]-acetic Acid tert-butyl Ester

To a stirred solution of the product of Step (1) (75.8 g, 285 mmol) in dimethylformamide (500 mL) at 0° C., was added sodium hydride (60% dispersion in oil, 13.7 g, 341 mmol). The mixture was stirred for 30 minutes and then a solution of o-bromobenzyl bromide (85.3 g, 341 mmol) in dimethylformamide (300 mL) was added slowly. The reaction mixture was stirred at 0° C. for 30 minutes and then allowed to warm to room temperature. After stirring for 16 hours, the reaction mixture was diluted with ether and water (1.5 L, 1:1), and the aqueous layer removed. The organic layer was washed with water (2×400 mL) and brine (1×400 mL), dried over sodium sulfate, filtered and concentrated. The crude material was purified by silica gel chromatography (eluant 95:5 hexane/ethyl acetate) to afford the title compound (75.5 g, 62%) as a yellow oil which solidified upon standing: ¹H NMR (300 MHz, CDCl₃) δ 1.39, 1.47 (2s, 9H), 3.89 (d, J=17 Hz, 2H), 4.70 (d, J=11 Hz, 2H), 5.19 (s, 2H), 7.11-7.58 (m, 9H); MS (APCI) m/z 434, 436 [M+H]⁺.

Step (3): Preparation of 1,3-dihydro-isoindole-1,2-dicarboxylic Acid 2-benzyl Ester 1-tert-butyl Ester

To an oven dried flask under an atmosphere of argon, was added Pd₂(dba)₃ (2.1 g, 2.3 mmol), 2-(diphenylphosphinyl)-2′-(N,N-dimethylamino)biphenyl (1.7 g, 4.6 mmol) and lithium tert-butoxide (12.2 g, 152 mmol). The flask was evacuated and purged with argon and the procedure repeated. Dioxane (150 mL) was added to the flask and the purging protocol repeated. This was followed by the addition of solution of the product of Step (2) (33.0 g, 76 mmol) in dioxane (100 mL). Once the addition was complete, the purging protocol was repeated and then the reaction mixture gradually heated to 60° C. over 1 hour. After stirring for a further 45 minutes at 60° C., the reaction mixture was cooled to room temperature, filtered through a pad of silica gel and washed with ether and the filtrate concentrated. The crude residue was purified by silica gel chromatography (eluant 90:10 hexane/ethyl acetate) to afford the title compound (8.7 g, 32%) as an orange oil: ¹H NMR (300 MHz, CDCl₃) δ 1.35, 1.50 (2s, 9H), 4.77-4.90 (m, 2H), 5.15-5.29 (m, 2H), 5.41-5.50 (m, 1H), 7.18-7.48 (m, 9H); MS (APCI) m/z 354 [M+H]⁺, 254 [M+H−100(Boc)]⁺.

Step (4): Preparation of octahydro-isoindole-1-carboxylic Acid tert-butyl Ester

To a solution of the product of Step (3) (4.3 g, 12 mmol) in methanol (250 mL) was added 10% Pd/C (0.8 g). The vessel was pressurized with hydrogen to 60 psi and shaken for 2 hours. The reaction mixture was then filtered through celite and the solvents evaporated. The residue was redissolved in ethanol (250 mL) and 5% Rh/C (1.6 g) and acetic acid (10 mL) were added. The vessel was pressurized with hydrogen to 60 psi and heated to 60° C. and shaken for 72 hours, maintaining the temperature and pressure. The reaction mixture was filtered through celite and the solvents removed in vacuo. The crude residue was purified by silica gel chromatography (eluant: ethyl acetate to ethyl acetate/methanol 95:5) to afford the title compound (1.1 g, 40%) as a colorless oil: ¹H NMR (300 MHz, CD₃OD) δ 1.05-1.78 (m, 8H), 1.48 (s, 9H), 2.23-2.35 (m, 1H), 2.40-2.51 (m, 1H), 2.97 (t, J=8.6 Hz, 1H), 3.12 (t, J=11.0 Hz, 1H), 3.88 (d, J=5.6 Hz, 1H); MS (APCI) m/z 226 [M+H]⁺.

Step (5): Preparation of octahydro-isoindole-1,2-dicarboxylic Acid 2-tert-butyl Ester

The product of Step (4) (2.47 g, 10.9 mmol) was partially dissolved in 4 M HCl (100 mL) and heated to reflux for 1.75 hours. At this time, mass spectroscopic analysis showed the reaction to be complete. The reaction mixture was cooled and the solvents evaporated in vacuo to afford the crude acid as a green solid. The crude acid was then dissolved in a mixture of dioxane and 1 M sodium hydroxide (1:1, 30 mL). Di-tert-butyl dicarbonate (“(Boc)₂O”) (3.1 g, 14 mmol) was added and the reaction mixture was stirred for 3.5 hours then diluted with ether (100 mL) and water (100 mL). The organic layer was removed and extracted with water (1×50 mL). The combined aqueous layers were diluted with methylene chloride (150 mL) and stirred vigorously whilst being acidified to pH 2 using 1 M HCl. The aqueous layer was then removed and extracted with methylene chloride (2×50 mL). The combined organic layers were dried over sodium sulfate, filtered and concentrated in vacuo to afford the title compound (2.1 g, 72%) as a light yellow solid: ¹H NMR (300 MHz, CD₃OD) δ 1.19-1.75 (m, 8H), 1.41, 1.45 (2s, 9H), 2.30-2.48 (m, 2H), 3.36 (dd, J=10.2, 5.8 Hz, 1H), 3.45 (dd, J=10.2, 7.8 Hz, 1H), 4.27 (d, J=7.0 Hz, 1H); MS (ESI) m/z 170 [M+H−100(Boc)]⁺.

Step (6): Preparation of 1-carbamoyl-octahydro-isoindole-2-carboxylic Acid tert-butyl Ester

To a stirred solution of the product of Step (5) (2.05 g, 7.6 mmol) in tetrahydrofuran (70 mL) at 0° C., were added 1-hydroxybenzotriazole (“HOBt”) (1.1 g, 8.4 mmol), N-methyl morpholine (0.9 mL, 8.4 mmol) and 1-(3-dimethylaminopropyl)-3-ethylcarbodiimide hydrochloride (“EDC”) (1.6 g, 8.4 mmol). The mixture was stirred for 1.75 hours, at which time concentrated ammonium hydroxide (1.0 mL, 15 mmol) was added. The reaction mixture was stirred for 18 hours and the solvents were evaporated. The residue was transferred to a separatory funnel using ethyl acetate (200 mL) and washed with 10% aqueous potassium bisulfate (50 mL). The resulting precipitate was collected by filtration to afford the desired product (1.31 g, 64%) as a white solid. The filtrate was then washed with saturated sodium bicarbonate (2×100 mL) and brine (1×100 mL), dried over sodium sulfate, filtered and concentrated to afford the title compound (0.5 g, 24%) as a white solid. The materials were combined to give an overall yield of 1.81 g, 89% of the desired product: ¹H NMR (300 MHz, CDCl₃) δ 1.14-1.72 (m, 8H), 1.45 (s, 9H), 2.28-2.47 (m, 2H), 3.38 (t, J=10.8 Hz, 1H), 3.58-3.71 (m, 1H), 4.21 (d, J=6.6 Hz, 1H), 5.42 (br s, 1H), 5.89 (br s, 1H); MS (APCI) m/z 269 [M+H]⁺, 169 [M+H−100(Boc)]⁺.

Step (7): Preparation of 1-cyano-octahydro-isoindole-2-carboxylic Acid tert-butyl Ester

To a stirred solution of the product from Step (6) (1.8 g, 6.7 mmol) in dimethylformamide (70 mL) at 0° C., was added cyanuric chloride (1.7 g, 9.4 mmol). The reaction mixture was stirred for 30 minutes, at which time a thick precipitate had developed. The mixture was quenched by pouring into a 1 M sodium hydroxide (100 mL) at 0° C. and then diluted with ethyl acetate (100 mL). The organic layer was removed and the aqueous layer extracted with ethyl acetate (2×50 mL). The combined organic layers were washed with brine (2×50 mL), dried over sodium sulfate, filtered and concentrated. The crude material was purified by silica gel chromatography (eluant, 90:10 ethyl acetate/hexanes to 80:20 ethyl acetate/hexanes) to afford the title compound (1.39 g, 84%) as a white solid: ¹H NMR (300 MHz, CDCl₃) δ 1.30-1.72 (m, 6H), 1.52 (s, 9H), 1.78-1.98 (m, 2H), 2.17-2.46 (m, 2H), 3.20-3.51 (m, 2H), 4.29-4.38 (m, 1H); MS (ESI) m/z 273 [M+H]⁺, 173 [M+H−100(Boc)]+.

Step (8): 1-(N-Hydroxycarbamimidoyl)-octahydro-isoindole-2-carboxylic Acid tert-butyl Ester

To a stirred solution of the product of Step (7) (1.38 g, 5.5 mmol) in ethanol (20 mL), was added 50% aqueous hydroxylamine (1.7 mL, 28 mmol). The reaction mixture was stirred for 16 hours at which time TLC analysis still showed the presence of starting material. An additional portion of hydroxylamine was added (0.5 mL) and the reaction mixture heated to 45° C. for two hours. The mixture was then cooled to room temperature and the solvents removed in vacuo. The residue was diluted with ether (30 mL) and water (30 mL). The resulting precipitate was collected by filtration to afford the title compound (1.37 g, 88%) as a white solid: ¹H NMR (300 MHz, CDCl₃) δ 1.09-1.77 (m, 8H), 1.42, 1.44 (2s, 9H), 2.23-2.45 (m, 2H), 3.31-3.38 (m, 1H), 3.56 (dd, J=9.8, 7.9 Hz, 1H), 4.19 (d, J=6.5 Hz, 1H); MS (ESI) m/z 284 [M+H]⁺.

Step (9): Preparation of 1-(5-oxo-4,5-dihydro-[1,2,4]oxadiazol-3-yl)-octahydro-isoindole-2-carboxylic acid-tert-butyl Ester

To a stirred solution of the product of Step (8) (1.34 g, 4.7 mmol) in anhydrous tetrahydrofuran (120 mL), was added carbonyl diimidazole (1.15 g, 7.1 mmol). The mixture was stirred at reflux for 3.5 hours, cooled to room temperature and the solvents evaporated in vacuo. The residue was dissolved in methylene chloride and extracted with 1 M NaOH (2×100 mL). The combined aqueous layers were acidified to pH 2 using 3 M HCl and then extracted with ethyl acetate (3×100 mL). The combined organic layers were dried over sodium sulfate, filtered and concentrated to afford the title compound (1.1 g, 76%) as a colorless oil: ¹H NMR (300 MHz, CDCl₃) δ 1.14-1.75 (m, 8H), 1.26 (br s, 9H), 2.38-2.52 (m, 2H), 3.32-3.48 (m, 1H), 3.54-3.68 (m, 1H), 4.73 (d, J=6.0 Hz, 1H); MS (EST) m/z 210 [M+H−100(Boc)]⁺.

Step (10): Synthesis of 3-(octahydro-isoindol-1-yl)-4H-[1,2,4]oxadiazol-5-one

To a stirred solution of the product of Step (9) (1.1 g, 3.6 mmol) in dioxane (30 mL) was added 2 M HCl in ether (15 mL).After stirring for 16 hours, starting material was still observed by TLC. A further portion of 2 M HCl in ether (20 mL) was added and the reaction mixture heated to reflux for 30 minutes. The reaction mixture was cooled to room temperature and concentrated in vacuo to a volume of approximately 20 mL, then diluted with ether. The precipitate was filtered to afford 3-(octahydro-isoindol-1-yl)₄H-[1,2,4]oxadiazol-5-one (0.7 g, 79%) as a white solid: ¹H NMR (300 MHz, CD₃OD) δ 1.05-1.91 (m, 8H), 2.55-2.64 (m, 1H), 2.71-2.86 (m, 1H), 3.28-3.39 (m, 1H), 3.50 (dd, J=9.7 Hz, 1H), 4.79 (d, J=5.3 Hz, 1H); MS (ESI) m/z 210 [M+H]⁺.

### COMPOUND EXAMPLE F2

### Synthesis of 3-(2-methyl-octahydro-isoindol-1-yl)-4H-[1,2,4]oxadiazol-5-one

To a suspension of the product of Compound Example F1 in water (10 mL) was added sodium carbonate (0.30 g, 2.8 mmol) and formalin (37% aqueous solution, 1.1 mL, 14 mmol). The mixture was stirred for 5 minutes before being added rapidly to a stirred solution of sodium borohydride (0.27 g, 7.1 mmol) in tetrahydrofuran (30 mL). The reaction was stirred for 1.5 hours and then concentrated to dryness. The residue was dissolved in methanol, reduced in volume to approximately 8 mL, and applied directly to a silica gel column (eluant 80:20 methylene chloride/methanol). The appropriate fractions were combined and found to contain a small amount of starting material. The residue was re-purified according to the same procedure to afford the desired product (415 mg, 65%) as a white solid: mp 102-104° C.; ¹H NMR (300 MHz, CD₃OD) δ 1.12-1.75 (m, 8H), 2.45-2.57 (m, 1H), 2.70-2.91 (m, 1H), 3.08 (dd, J=9.9, 9.7 Hz, 1H), 3.52 (dd, J=11.0, 10.8 Hz, 1H), 4.20 (d, J=5.6 Hz, 1H); ¹³C NMR (CD₃OD, 75 MHz) δ 19.8, 22.2, 23.4, 23.7, 35.6, 41.5, 41.8, 56.7, 69.1, 162.7, 171.5; MS (ESI) m/z 224 [M+H]⁺. Anal. Calcd. for C₁₁H₁₇N₃O₂.0.75H₂O: C, 55.80; H, 7.87; N, 17.75. Found: C, 55.81; H, 7.84; N, 7.88.

Compounds of Formula I wherein n is 1, each of Y⁴ to Y⁷ is C(R¹⁰)R^{10w}, and at least one R¹⁰ is not H may be prepared by conventional means according to the method illustrated below in Scheme G.

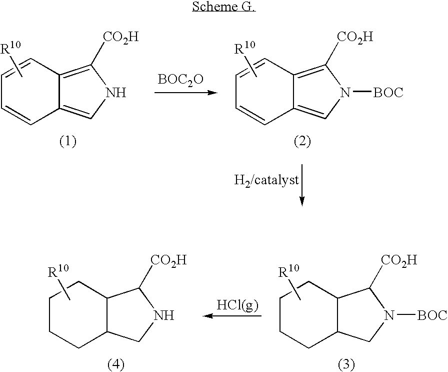

Alternatively, compounds of formula (4) may be prepared as illustrated below in Scheme N.

A compound of Formula I wherein n is 1 and R³ is not H may be prepared by conventional means as illustrated below in Scheme H.

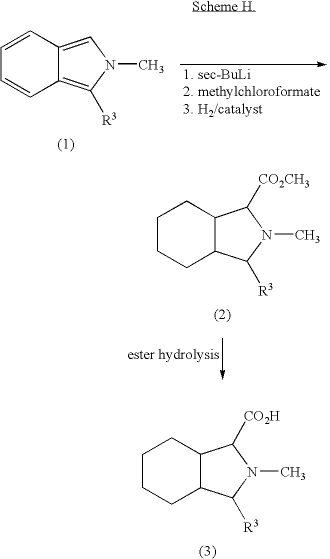

Compounds of Formula I wherein n is 1 and at least one of Y⁴ is a heteroatom may be prepared by conventional means as illustrated below in Scheme I.

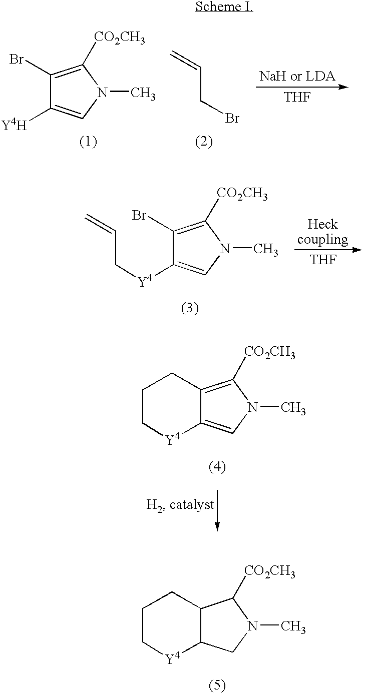

Alternatively, a compound of Formula I wherein Y⁴ is O may be prepared as illustrated below in Scheme Q.

Compounds of Formula I wherein n is I and at least one of Y⁵ is a heteroatom may be prepared by conventional means as illustrated below in Scheme J.

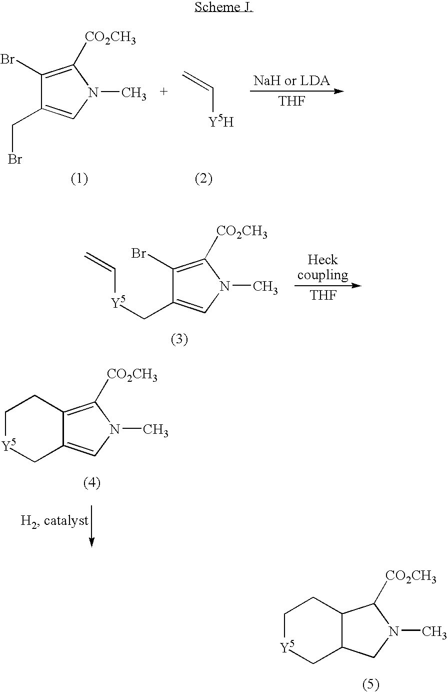

Compounds of Formula I wherein n is 1 and at least one of Y⁶ is a heteroatom may be prepared by conventional means as illustrated below in Scheme K.

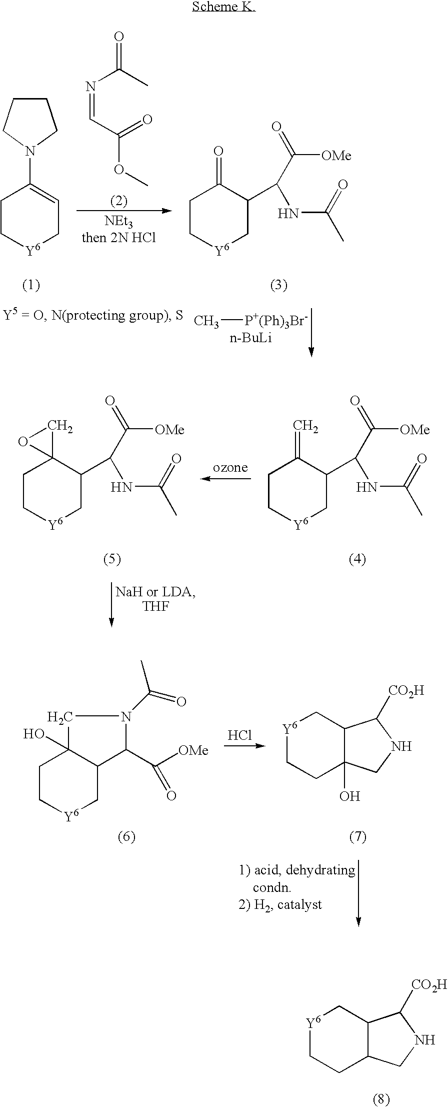

Compounds of Formula I wherein n is 1 and at least one of Y⁷ is a heteroatom may be prepared by conventional means as illustrated below in Scheme L.

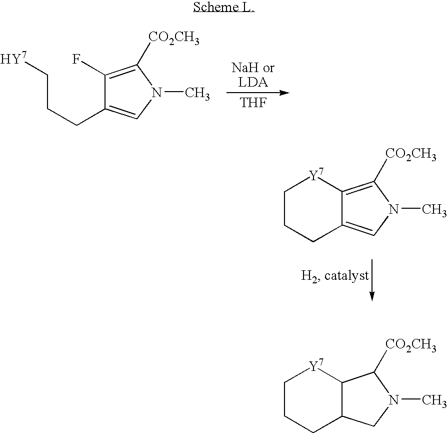

It should be appreciated that while Schemes A, B, and D to F, I, J, K, and L illustrate preparations of compounds of Formula I wherein n is 1, a person of ordinary skill in the art will know how to adapt the teachings of these schemes to prepare compounds of Formula I wherein n is 0 or 2.

Compounds of Formula I wherein any two groups as identified above for Formula I are taken together with the ring atoms of Formula I to which they are attached to form a fused ring may be prepared by conventional means as illustrated below in Scheme M.

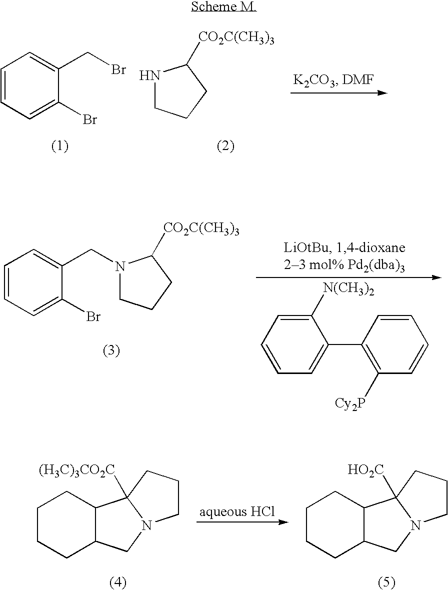

Alternatively, the method illustrated below in Scheme Q may be used to prepare ring-fused compounds.

A compound of Formula I was prepared according to the method illustrated above in Scheme M as described below in Compound Example M1.

### COMPOUND EXAMPLE M1

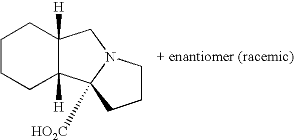

### Synthesis of a Racemic Mixture of (5aS,9aR,9bS)-octahydro-pyrrolo[2,1-a]isoindole-9b-carboxylic Acid Hydrochloride and (5aR,9aS,9bR)-octahydro-pyrrolo[2,1-a]isoindole-9b-carboxylic Acid Hydrochloride

Step (1): Preparation of octahydro-pyrrolo[2,1-a]isoindole-9b-carboxylic Acid tert-butyl Ester

To a Parr bottle containing 5% Rh/C (0.4 g) was added ethanol (50 mL) under an atmosphere of nitrogen. The mixture was shaken with hydrogen (40 psi) for 20 minutes to pre-reduce the catalyst. A solution of 2,3-dihydro-1H, 5H-pyrrolo[2,1-a]isoindole-9b-carboxylic acid tert-butyl ester (Buchwald, S. L. et al. J. Org. Chem. 2002, 67, 465-475.2.0 g, 7.72 mmol) in ethanol (130 mL) was added to the pre-reduced catalyst and the reaction mixture shaken at 60° C. for 3 days under hydrogen (60 psi). The mixture was filtered through a pad of celite and concentrated. Silica gel chromatography using hexanes/ethyl acetate (1:1) as eluant afforded the title compound (0.6 g, 30%) as a colorless oil: ¹H NMR (300 MHz, CD₃OD) δ 1.20-1.46 (m, 4H), 1.50 (s, 9H), 1.63-1.75 (m, 5H), 1.99-2.04 (m, 2H), 2.27-2.29 (m, 1H), 2.36-2.38 (m, 1H), 2.68-2.70 (m, 1H), 2.88 (dd, J=11.7, 8.0 Hz, 1H), 3.06-3.12 (m, 1H), 3.45-3.58 (m, 2H); MS (ESI) m/z 266 [M+H]⁺.

Step (2): Synthesis of a Racemic Mixture of (5aS,9aR,9bS)-octahydro-pyrrolo[2,1-a]isoindole-9b-carboxylic Acid Hydrochloride and (5aR,9aS,9bR)-octahydro-pyrrolo[2,1-a]isoindole-9b-carboxylic Acid Hydrochloride

A mixture of the compound from Step (1) (0.6 g, 2.26 mmol) and 20% aqueous HCl (20 mL) was heated to reflux for 2 hours. The reaction mixture was allowed to cool to room temperature, and concentrated to dryness. The white solid was stirred in ether (20 mL) and acetonitrile (2 mL) for 3 hours, and the product was collected by filtration and dried (0.4 g, 72%): mp: 250-255° C.; ¹H NMR (300 MHz, CD₃OD) δ 1.24-1.34 (m, 3H), 1.54-1.55 (m, 1H), 1.69-1.78 (m, 4H), 1.93-1.94 (m, 1H), 2.15-2.26 (m, 2H), 2.44-2.45 (m, 1H), 2.61-2.66 (m, 1H), 2.90-2.91 (m, 1H), 3.22 (dd, J=18.6, 11.0 Hz, 1H), 3.34-3.36 (m, 1H), 3.70 (t, J=12.1 Hz, 1H), 3.76-3.77 (m, 1H); ¹³C NMR (75 MHz, CD₃OD) δ 21.1, 24.3, 24.5, 25.0, 25.3, 36.5, 36.8, 46.1, 57.3, 58.7, 87.9, 172.4; MS (ESI) m/z 210 [M+H]⁺.

Anal. Calcd. for C₁₂H₁₉NO₂.1.05HCl-0.3H₂O: C, 56.97; H, 8.23; N, 5.54; Cl, 14.72. Found: C, 57.12; H, 8.12; N, 5.50; Cl, 14.52; relative stereochemistry was determined by Nuclear Overhauser Effect nuclear magnetic resonance experiment.

In addition to the method outlined above for Scheme G, compounds of Formula I wherein n is 1, each of Y⁴ to Y⁷ is C(R°)R^{10w}, and at least one R¹⁰ is not H may be prepared by conventional means according to the method illustrated below in Scheme N.

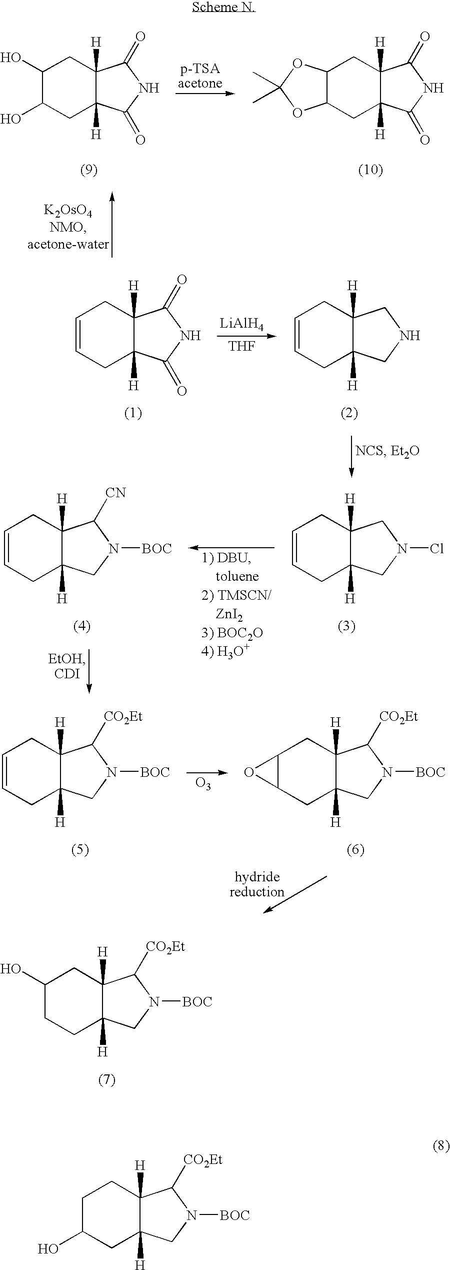

Compounds of Formula I wherein n is 1, each of Y⁴ to Y⁷ is C(R¹⁰)R^{10w}, and at least one R¹⁰ is not H may be prepared by conventional means starting with a compound of formulas (7) or (8) from Scheme N. For example, the hydroxy in a compound of formulas (7) or (8) may be displaced with a nucleophile using Mitsunobu conditions or via a corresponding bromo intermediate, for example. Alternatively, the hydroxy can be oxidized to the corresponding ketones, which are useful for reaction under Wittig conditions or with aryl lithium agents in the presence of titanium tetrachloride at temperatures of from about −100° C. to about 0° C. in a suitable solvent such as dichloromethane to give the corresponding phenyl-hydroxy geminally disubstituted compound. The aryl- or heteroaryl-hydroxy geminally substituted compound is a compound of Formula I wherein R¹⁰ is aryl or heteroaryl and R^{10w} is HO. Compounds such as the phenyl-hydroxy geminally substituted compound is a benzylic-type alcohol which may readily be eliminated to give a mixture of two regioisomeric phenyl-substituted cycloalkenes. The regioisomeric aryl- or heteroaryl-substituted cycloalkenes such as the phenyl-substituted cycloalkenes may be reduced to give the phenyl-substituted compounds, which are compounds of Formula I wherein R¹⁰ is phenyl (aryl or heteroaryl).

Alternatively, the corresponding ketones of a compound of formulas (7) or (8) discussed above may be deprotonated to form an enolate on either side, and the enolate allowed to react with an electrophile to provide each to two regioisomeric alpha substituted ketones. The alpha substituted ketones can be reduced under conventional conditions to convert the carbonyl of the ketone to a CH₂ group.

Compounds of Formula I have been prepared in the manner illustrated above in Scheme N as described below in Compound Examples N1 to N4.

### COMPOUND EXAMPLE N1

Synthesis of 5,6-Dimethoxy-octahydro-isoindole-1-carboxylic Acid

Step (1): Preparation of 1-cyano-5,6-Dihydroxy-octahydro-isoindole-2-carboxylic Acid tert-butyl Ester

The 2,2-dimethyl-hexahydro-[1,3]dioxolo[4,5-f]isoindole-5,7-dione (prepared as described below for Compound Example N 4, 2.05 g, 6.3 mmol) was suspended in THF (30 mL) and 1 M HCl (30 mL) was added to it. The mixture was stirred at ambient temperature overnight. The reaction was basified to pH 9-10 with 1 M NaOH and dioxane (50 mL) was added followed by BOC₂O (1.5 g, 7 mmol). After stirring overnight, the reaction mixture was extracted with EtOAc (3×100 mL). The organic layers were combined, dried over anhydrous Na₂SO₄, filtered and concentrated to afford the desired product (1.7 g, 90%) as a white solid: ¹H NMR (300 MHz, CD₃OD) δ 1.5 (2s, 9H), 1.6-1.9 (m, 4H), 2.5 (m, 2H), 3.45 (m, 1H), 3.92 (m, 2H), 4.1-4.3 (m, 1H), 4.8 (m, 1H); MS (ESI) m/z 283 [M+H]⁺.

Step (2): Preparation of 1-cyano-5,6-Dimethoxy-octahydro-isoindole-2-carboxylic Acid tert-butyl Ester

To a suspension of diol the product of Step (1) (1.8 g, 6.4 mmol) in DMF (50 mL) and CH₃I (3.6 g, 25.5 mmol), cooled to 0° C. was added NaH (60% in mineral oil; 0.64 g, 16.0 mmol) in one portion. The reaction mixture was stirred at 5° C. for 2 hours, diluted with ethyl acetate (200 mL), and washed with water (5×40 mL) and brine (2×50 mL). The organic layer was dried over anhydrous Na₂SO₄, filtered and concentrated on a rotary evaporator. The crude residue was purified by column chromatography to afford the desired product (1.3 g, 65%) as a thick oil: ¹H NMR (300 MHz, CDCl₃) δ 1.4-1.8 (m, 1H), 2.05 (m, 1H), 2.2-2.7 (m, 4H), 3.2-3.6 (m, 8H), 3.9 (m, 1H), 4.8 (m, 1H); MS (ESI) m/z 311 [M+H]⁺.

Step (3): Synthesis of 5,6-Dimethoxy-octahydro-isoindole-1-carboxylic Acid

In a manner similar to that described below for Compound Example N 4, the product of Step (2) was converted to the title compound, a tan solid: mp 235-241 C (dec); ¹H NMR (300 MHz, CD₃OD) δ 1.2-1.5 (m, 2H), 1.7-2.1 (m, 3H), 2.5-2.7 (m, 2H), 3.2 (m, 1H), 3.2-3.38 (4s, 6H), 3.6 (m, 1H), 4.07 (d, J=6.1 Hz, 1H); ¹³C NMR (75 MHz, CD₃OD) δ 172.0, 77.7, 76.9, 76.0, 67.3, 65.5, 57.5, 57.2, 56.8, 41.8, 39.2, 36.5, 36.3, 28.0, 27.0, 25.6, 24.9, MS (ESI) m/z 230 [M+H]⁺.

### COMPOUND EXAMPLE N2

### Synthesis of cis-2,3,3a,4,7,7a-Hexahydro-1H-isoindole-1-carboxylic Acid Hydrochloride

The cis-1,3,3a,4,7,7a-hexahydro-isoindole (Keinan, E. et al. J. Am. Chem. Soc. 2000; 122:10743-10753, 10.0 g, 81.1 mmol) was converted to the title compound in a manner similar to that described below in Compound Example N4, Steps (4) to (6) to afford the desired product (0.53 g, 84%) as a white solid: mp 217-220° C.; ¹H NMR (300 MHz, D₂O) δ 1.76-1.85 (m, 1H), 2.04-2.38 (m, 3H), 2.46-2.59 (m, 2H), 3.07 (dd, J=11.5, 6.2 Hz, 1H), 3.46 (dd, J=11.4, 6.3 Hz, 1H), 3.95 (d, J=7.1 Hz, 1H), 5.66 (s, 2H); ¹³C NMR (75 MHz, D₂O) δ 172.5, 124.8, 124.2, 63.8, 50.7, 39.4, 33.9, 23.8, 23.4, MS (ESI) m/z 168 [M+H]⁺.

Anal. Calcd. for C₉H₁₃NO₂—HCl: C, 53.08; H, 6.93; N, 6.88; Cl, 17.41. Found: C, 52.83; H, 7.06; N, 6.75; Cl, 17.19.

### COMPOUND EXAMPLE N3

### Synthesis of Diastereomer 1 of 6-chloro-2,2-dimethyl-octahydro-[1,3]dioxolo[4,5-f]isoindole-5-carboxylic Acid

In a manner similar to that described below for Compound Example N4, Step (6), diastereomer 1 of Compound Example N4, Step (5) was converted to the title compound in 74% yield: mp 257-262° C. (dec); ¹H NMR (300 MHz, D₂O) δ 1.32 (dt, J=12, 2 Hz, 1H), 1.63-1.86 (m, 3H), 2.69-2.84 (m, 2H), 3.21 (t, J=12 Hz, 1H), 3.40 (m, 1H), 3.73 (m, 1H), 3.89 (t, J=2.1 Hz, 1H), 4.34 (d, J=6 Hz, 1H); ¹³C NMR (75 MHz, D₂O) δ 170.5, 68.2, 66.3, 64.2, 46.7, 37.3, 34.5, 26.7, 24.9, MS (ESI) m/z 202 [M+H]⁺. Anal. Calcd. for C₉H₁₅NO₄.1.05HCl-0.3H₂O: C, 44.14, H, 6.74, N, 5.72, Cl, 15.20. Found: C, 44.16, H, 6.74, N, 5.59, Cl, 15.39.

### COMPOUND EXAMPLE N4

### Synthesis of Diastereomer 2 of 6-chloro-2,2-dimethyl-octahydro-[1,3]dioxolo[4,5-f]isoindole-5-carboxylic Acid

Step (1): Preparation of 5,6-dihydroxy-hexahydro-isoindole-1,3-dione

To a solution of tetrahydrophthalimide (20.0 g, 132 mmol) in acetone (200 mL) and water (40 mL) was added N-methylmorpholine-N-oxide (16.3 g, 139 mmol). The mixture was cooled to 0° C. and K₂OsO₄-2H₂O (0.15 g, 0.4 mmol) was added. The reaction mixture was allowed to warm to ambient temperature and stirred overnight. The precipitated solids were filtered, washed with acetone (100 mL) and dried in a vacuum oven to afford the desired product (19.2 g, 79%) as a white solid: ¹H NMR (300 MHz, DMSO-d₆) δ 1.64 (m, 2H), 1.84 (m, 2H), 2.91 (s, 2H), 3.4 (s, 2H), 4.6 (s, 2H), 10.8 (s, 1H); MS (APCI) m/z 184 [M−H]⁻.
Step (2): Preparation of 2,2-dimethyl-hexahydro-[1,3]dioxolo[4,5-f]isoindole-5,7-dione

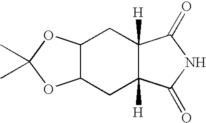

To a suspension of the diol the product from Step (1) (20.0 g, 108 mmol) in acetone (400 mL) was added p-toluenesulfonic acid (“p-TSA”), 2.0 g; mmol). The suspension was stirred at ambient temperature overnight. The reaction was then concentrated in vacuo. The residue was diluted with ethyl acetate (400 mL) and washed with water (125 mL) and brine (200 mL), dried over anhydrous Na₂SO₄, filtered and concentrated in vacuo to afford the title compound (21.0 g, 86%) as a white solid: ¹H NMR (300 MHz, CD₃OD) δ 1.32 (s, 3H), 1.42 (s, 3H), 1.57 (t, J=12.3 Hz, 2H), 2.15 (dd, J=14.3, 3.3 Hz, 2H), 3.04 (m, 2H), 4.45 (s, 2H); MS (APCI) m/z 226 [M+H]⁺.

Step (3): Preparation of 2,2-dimethyl-octahydro-[1,3]dioxolo[4,5-f]isoindole

Lithium aluminum hydride (11.8 g, 310.8 mmol) was suspended in THF (310 mL) and cooled to 0° C. A solution of the product of Step (2) (20.0 g, 88.80 mmol) in THF (190 mL) was added dropwise to this suspension and the reaction mixture heated to reflux. After refluxing overnight, the reaction was cooled to ambient temperature and then to 0° C. Water (12 mL) was added carefully and the mixture was stirred for 5 minutes. To this mixture was added a 15% aqueous solution of NaOH (12 mL). After stirring for 5 minutes another portion of water (36 mL) was added. The mixture was stirred for 30 minutes, dried over anhydrous Na₂SO₄, filtered and concentrated in vacuo to afford the title compound (17.0 g, 97%) as a white solid: ¹H NMR (300 MHz, DMSO-d₆) δ 1.15 (m, 2H), 1.23 (s, 3H), 1.34 (s, 3H), 1.69 (d, J=3.2 Hz, 1H), 1.74 (d, J=3.2 Hz, 2H), 2.1 (m, 2H), 2.09 (dd, J=12, 5.6 Hz, 2H), 2.25 (dd, J=10.8, 5.0 Hz, 2H), 2.90 (dd, J=10.9, 7.3 Hz, 2H), 4.30 (s, 2H); MS (APCI) m/z 198 [M−H]⁻.

Step (4): Preparation of 6-chloro-2,2-dimethyl-octahydro-[1,3]dioxolo[4,5-f]isoindole

To a solution of the product of Step (3) (10.0 g, 50.7 mmol) in ether (300 mL) was added N-chlorosuccinimide (7.11 g, 53.23 mmol). The reaction mixture was stirred for 3 hours at ambient temperature, diluted with ether (100 mL), washed with 1 M HCl (75 mL) and brine (75 mL). The organic layer was dried over anhydrous Na₂SO₄, filtered and concentrated in vacuo to afford the title compound (11.2 g, 95%) as a white solid: ¹H NMR (300 MHz, CDCl₃) δ 1.35 (m, 2H), 1.39 (s, 3H), 1.45 (s, 3H), 1.91 (d, J=3.0 Hz, 2H), 1.96 (d, J=3 Hz, 2H), 2.52 (m, 2H), 2.82 (dd, J=9.7, 5.3 Hz, 2H), 3.36 (dd, J=9.6, 7.3 Hz, 2H), 4.40 (s, 2H); MS (ESI) m/z 232 [M+H]⁺.

Step (5): Preparation of 6-chloro-2,2-dimethyl-octahydro-[1,3]dioxolo[4,5-f]isoindole-5-carbonitrile

The product of Step (4) (11.1 g, 47.91 mmol) was dissolved in toluene (300 mL) and to it 1,8-diazabicyclo[5.4.0]undec-7-ene (“DBU”) (10.94 g, 71.86 mmol) was added. After stirring at ambient temperature for 66 hours, the reaction mixture was washed with water (4×75 mL). The organic layer was dried over anhydrous Na₂SO₄ and filtered. Trimethylsilyl cyanide (“TMSCN”) (15.2 g, 153.31 mmol) and ZnI₂ (0.92 g, 2.88 mmol) were added to this filtrate and the mixture was stirred at ambient temperature for 2 hours. After 2 hours, the reaction mixture was poured into a 1:1 solution of water and dioxane (400 mL) and stirred for 1 hour. The layers were separated and the aqueous layer was washed with ethyl acetate (2×250 mL). The organic layers were combined, dried over anhydrous Na₂SO₄, filtered and concentrated in vacuo. The residue was dissolved in CH₂Cl₂ (300 mL) and Boc₂O (7.0 g, 32.1 mmol) was added. The mixture was stirred at ambient temperature overnight. The reaction mixture was washed with 1 M HCl (100 mL) followed by water (100 mL) and brine (100 mL). The organic layer was dried over anhydrous Na₂SO₄, filtered and concentrated in vacuo. The crude residue was purified by column chromatography using a gradient of ethyl acetate/hexanes (5-40%) to afford the title compound as diastereomer 1 (top spot; 1.12 g, 7.3%): ¹H NMR (300 MHz, CDCl₃) δ 1.2-1.6 (m, 2H), 1.45 (s, 6H), 1.49 (s, 9H), 1.70-1.78 (m, 1H), 1.95 (m, 1H), 2.04-2.18.(m, 1H), 2.60-2.79 (m, 2H), 3.0 (dd, J=11.1, 7.1 Hz, 1H), 3.94 (t, J=9.6 Hz, 1H), 4.41-4.54 (m 2H); MS (ESI) m/z 323 [M+H]⁺; and diastereomer 2 (lower spot; 2.10 g, 13.6%): ¹H NMR (300 MHz, CDCl₃) δ 1.23-1.38 (m, 2H), 1.26 (s, 3H), 1.33 (s, 12H), 2.05 (m, 3H), 2.71-2.85 (m, 2H), 3.23-3.25 (m, 1H), 3.67 (t, J=9.5 Hz, 1H), 4.37 (br s, 2H); MS (ESI) m/z 323 [M+H]⁺.

Step (6): Synthesis of Diastereomer 2 of 6-chloro-2,2-dimethyl-octahydro-[1,3]dioxolo[4,5-f]isoindole-5-carboxylic Acid

The diastereomer 2 from Step (5) was suspended in 6 M HCl and heated to reflux for 16 hours. The reaction mixture was cooled to ambient temperature and concentrated in vacuo. The residue was then triturated in Et₂O for 30 minutes, filtered and dried in a vacuum oven at 50° C. overnight to afford the title compound in 52% yield: mp: 236-244° C. (dec); ¹H NMR (300 MHz, D₂O) δ 1.47-1.55 (m, 1H), 1.70-1.97 (m, 3H), 2.51-2.65 (m, 2H), 3.12-3.48 (m, 2H), 3.82 (br s, 2H), 4.05 (d, J=6.8 Hz, 1H); ³C NMR (75 MHz, D₂O) δ 172.2, 67.6, 67.2, 62.2, 49.4, 40.1, 33.9, 28.1, 27.8, MS (ESI) m/z 202 [M+H]⁺. Anal. Calcd. for C₉H₁₅NO₄-1.1HCl-0.15H₂O: C, 44.30; H, 6.77; N, 5.74; Cl, 15.98. Found: C, 44.54; H, 6.88, N, 5.74; Cl, 15.79.

Compounds of Formula I wherein any two groups as identified above for Formula I are taken together with the ring atoms of Formula I to which they are attached to form a fused ring may be prepared by conventional means as illustrated below in Scheme O.

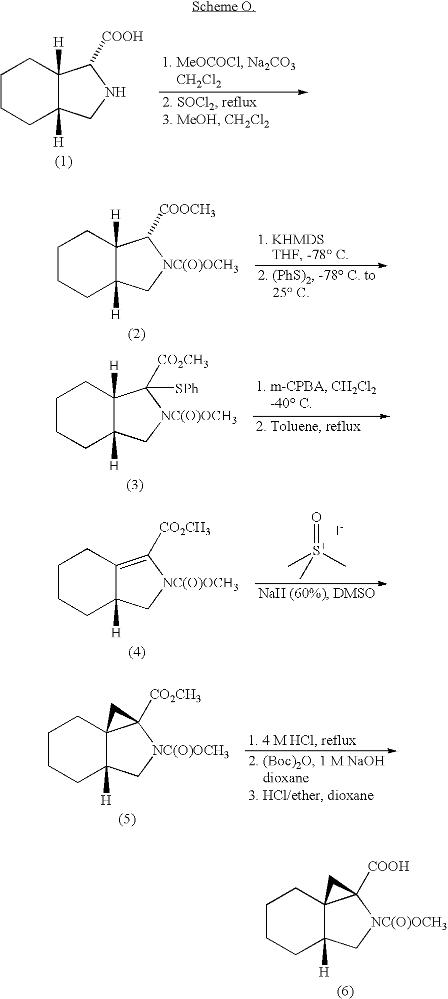

Alternatively, compounds of Formula I wherein R^{3a} or R^{7a} is not H may be prepared by conventional means as illustrated below in Scheme P.

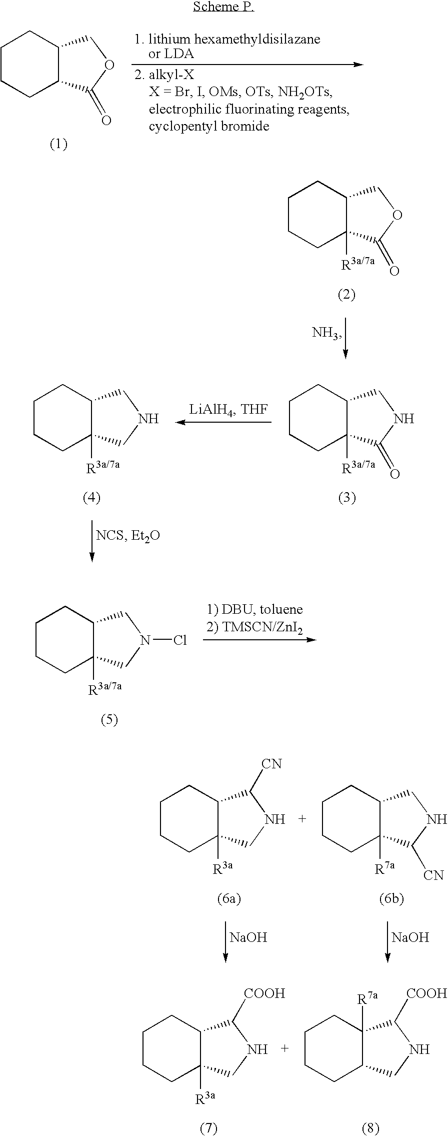

A compound of Formula I wherein Y⁴ is O may be prepared as illustrated below in Scheme Q.

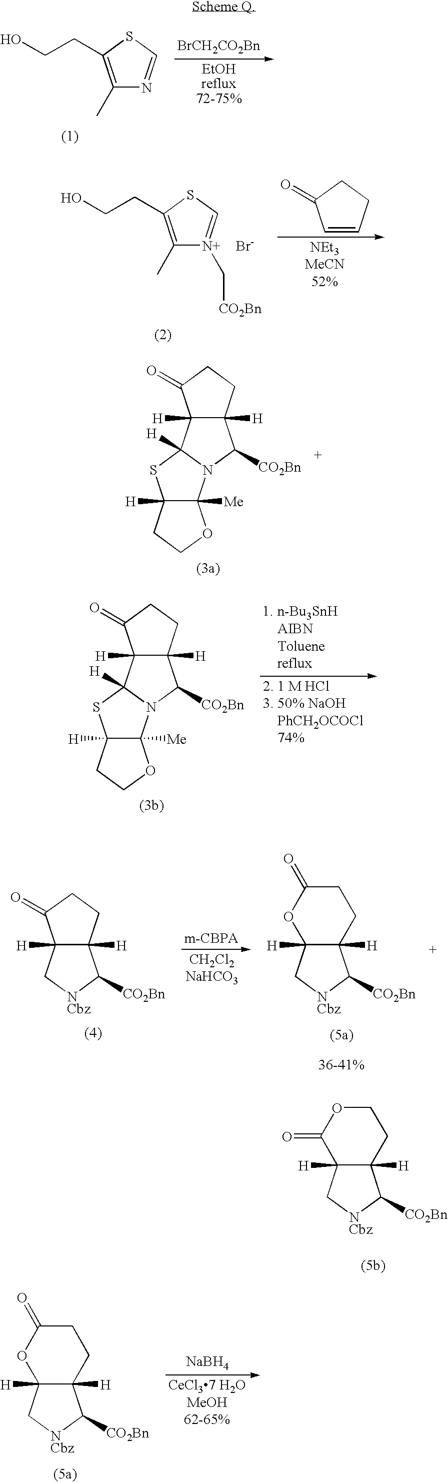

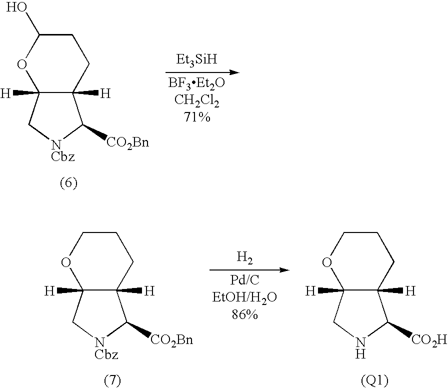

Compounds of Formula I have been prepared in the manner illustrated above in Scheme Q as described below in Compound Examples Q1 to Q6.

### COMPOUND EXAMPLE Q1

### Synthesis of 3-aza-6-oxabicyclo[4.3.0]nonane-2-carboxylic Acid

Step (1): Preparation of 3-benzyloxycarbonylmethyl-5-(2-hydroxyethyl)-4-methylthiazolium Bromide.

A solution of 4-methyl-5-thiazole ethanol (27.7 g, 194 mmol) and benzyl bromoacetate (44.3 g, 193 mmol) in absolute ethanol (110 mL) was heated to reflux for 8 hours. The solution was cooled, and the solvent was removed in vacuo. Trituration with 2-propanol followed by collection of the resulting solid afforded 3-benzyloxycarbonylmethyl-5-(2-hydroxyethyl)-1 methylthiazolium bromide (54.3 g, 75%) as a white solid: ¹H NMR (300 MHz, DMSO-d₆) δ 10.08 (s, 1H), 7.40-7.42 (m, 5H), 5.70 (s, 2H), 5.27 (s, 2H), 3.65 (t, J=5.6 Hz, 2H), 3.05 (t, J=5.6 Hz, 2H), 2.37 (s, 3H); MS (ESI) m/z 292 [C₁₅H₁₈NO₃S+H]⁺.
Step (2): Preparation of Benzyl 3a-methyl-7-oxodecahydro-3-oxa-8-thia-3b-azadicyclopenta[a,e]pentalene-4-carboxylate

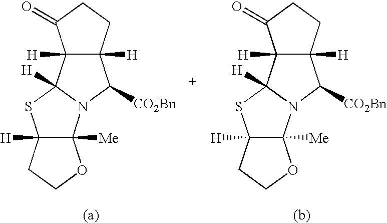

To a stirred suspension of 3-benzyloxycarbonylmethyl-5-(2-hydroxyethyl)-4-methylthiazolium bromide (50 g, 134 mmol) and 2-cyclopentenone (50 g, 609 mmol) in acetonitrile (250 mL) was added triethylamine (14.9 g, 147 mmol) drop-wise over 15 minutes. After the suspension was stirred for 24 hours, brine (100 mL) and isopropyl acetate (100 mL) were added. The layers were separated, and the aqueous layer was extracted with isopropyl acetate (2×75 mL). The organic layers were combined, dried over sodium sulfate, filtered and concentrated in vacuo. The residual solid was recrystallized twice from hexanes/ethyl acetate to afford two crops of benzyl 3a-methyl-7-oxodecahydro-3-oxa-8-thia-3b-azadicyclopenta[a,e]pentalene-4-carboxylate (20.9 g, 42%). The mother liquors were combined, concentrated and chromatographed (silica gel, 75:25 hexanes/ethyl acetate) to afford an 80:20 mixture of isomers (a) and (b) (total yield 26.3 g, 52%) as a white solid; isomer (a): ¹H NMR (300 MHz, CDCl₃) δ 7.34-7.39 (m, 5H), 5.17-5.28 (m, 3H), 4.02-4.10 (m, 2H), 3.84-3.87 (m, 2H), 3.13-3.17 (m, 2H), 2.55-2.62 (m, 1H), 1.84-2.35 (m, 5H), 1.50 (s, 3H); MS (ESI) m/z 374 [C₂₀H₂₃NO₄S+H]⁺; 80:20 mixture of isomers (a) and (b): ¹H NMR (300 MHz, CDCl₃) δ 7.34-7.39 (m, 5H), 5.43 (d, J=7.6 Hz, 0.2H), 5.17-5.28 (m, 2.8H), 4.00-4.13 (m, 2H), 3.96 (td, J=8.1, 2.0 Hz, 0.2H), 3.82-3.87 (m, 1.6H), 3.71 (d, J=5.1 Hz, 0.2H), 3.52 (d, J=7.3 Hz, 0.2H), 3.33 (dd, J=9.5, 8.6 Hz, 0.2H), 3.13-3.17 (m, 1.6H), 2.55-2.62 (m, 0.8H), 1.84-2.35 (m, 5.2H), 1.50 (s, 2.4H), 1.36 (s, 0.6H).

Step (3): Preparation of benzyl N-(benzyloxycarbonyl)-3-aza-6-oxobicyclo[3.3.0]octane-2-carboxylate

A stirred suspension of benzyl 3a-methyl-7-oxodecahydro-3-oxa-8-thia-3b-azadicyclopenta[a,e]pentalene-4-carboxylate isomers (a) and (b) from Step (2) (11.0 g, 29.5 mmol), 2,2′-azobisisobutyronitrile (“AIBN”) (0.72 g, 4.4 mmol) and tri-n-butyltin hydride (11.3 g, 39.0 mmol) in toluene (90 mL) was heated to reflux. After 7.5 hours, thin layer chromatography (“TLC”) analysis indicated that the starting material was consumed. The solution was cooled and concentrated in vacuo, and the residual viscous orange oil was dissolved in ether (90 mL). A solution of 1 M HCl (36 mL) was added with vigorous stirring. After 16 hours, the layers were separated, and the aqueous layer mixed with isopropyl acetate (100 mL). The mixture was cooled to −5° C., and benzyl chloroformate (5.9 g, 34.8 mmol) was added in a single portion followed by the dropwise addition of 50% NaOH (5.3 mL). After complete addition (exotherm to 7° C.), the mixture was stirred at 0-5° C. for 3 hours. The layers were separated, and the aqueous layer was extracted with iso-propyl acetate (4×75 mL). The organic layers were combined, washed with brine (50 mL), dried over sodium sulfate, filtered, and concentrated in vacuo. The crude product was chromatographed (silica gel, 80:20 to 70:30 hexanes/ethyl acetate) to afford benzyl N-(benzyloxycarbonyl)-3-aza-6-oxobicyclo[3.3.0]octane-2-carboxylate (8.5 g, 74%) as a colorless oil: ¹H NMR (300 MHz, CDCl₃) δ 7.32-7.35 (m, 10H), 5.21 and 5.13 (s, AB q, J=12.3 Hz, 2H), 5.05 and 5.03 (s, AB q, J=12.2 Hz, 2H), 4.32 and 4.24 (2 d, J=3.8, 3.6 Hz, 1H), 3.78-3.84 (m, 2H), 2.99-3.07 (m, 1H), 2.79-2.84 (m, 1H), 2.25-2.40 (m, 3H), 1.90-2.02 (m, 1H); MS (ESI) m/z 394 [C₂₃H₂₃NO₅+H]⁺.

Step (4): Preparation of Benzyl N-benzyloxycarbonyl)-3-aza-7-oxo-6-oxabicyclo[4.3.0]nonane-2-carboxylate

To a stirred suspension of benzyl N-benzyloxycarbonyl)-3-aza-6-oxobicyclo[3.3.0]octane-2-carboxylate (7.3 g, 18.6 mmol) and sodium bicarbonate (2.3 g, 27.4 mmol) in dichloromethane (200 mL) was added meta-chloroperbenzoic acid (“mCPBA”) (77%, 7.1 g, 31.5 mmol) in a single portion. The mixture was stirred for 4 days, at which time TLC analysis indicated that most of the starting material was consumed. A 5% solution of sodium bisulfite (100 mL) was added. After stirring for 3 hours, a saturated sodium bicarbonate solution (100 mL) was added, and the layers were separated. The organic layer was washed with sodium bicarbonate (2×75 mL) and brine (75 mL), dried over sodium sulfate, and filtered through celite. The filtrate was concentrated, and the residue chromatographed (silica gel, 80:20 to 67:33 hexanes/ethyl acetate) to afford unreacted starting material (1.7 g, 23%), benzyl N-(benzyloxycarbonyl)-3-aza-6-oxo-7-oxabicyclo[4.3.0]nonane-2-carboxylate (0.99 g, 13%) as a white solid. Also obtained was desired benzyl N-(benzyloxycarbonyl)₃-aza-7-oxo oxabicyclo[4.3.0]nonane-2-carboxylate (2.7 g, 36%) as a colorless oil: ¹H NMR (300 MHz, CDCl₃) δ 7.24-7.35 (m, 10H), 5.20 and 5.15 (AB q, s, J=12.4 Hz, 2H), 5.04 (s, 2H), 4.44 and 4.40 (2 t, J=3.9, 3.9 Hz, 1H), 4.20-4.31 (m, 2H), 3.89-4.03 (m, 2H), 3.22-3.30 (m, 1H), 2.75-2.90 (m, 1H), 2.08-2.13 (m, 1H), 1.80-1.93 (m, 1H); MS (ESI) m/z 410 [C₂₃H₂₃NO₆+H]⁺.

Step (5): Preparation of Benzyl N-(benzyloxycarbonyl)₃-aza-7-hydroxy-6-oxabicyclo[4.3.0]nonane-2-carboxylate

To an ice-cold solution of benzyl N-(benzyloxycarbonyl)₃-aza-7-oxo-6-oxabicyclo[4.3.0]nonane-2-carboxylate (5.0 g, 12.3 mmol) and cerium (if) chloride heptahydrate (6.5 g, 17.3 mmol) in methanol (110 mL) was added sodium borohydride (0.25 g, 6.6 mmol) as a solid in small portions over 15 minutes. At complete addition the ice bath was removed, and the solution was allowed to warm to room temperature. After one hour, TLC analysis indicated that the starting material was consumed. The solution was transferred to a separatory funnel with water (100 mL) and ethyl acetate (100 mL). The layers were separated, and the aqueous layer was extracted with ethyl acetate (50 mL). The organic layers were combined, washed with brine (50 mL), dried over sodium sulfate, filtered and concentrated in vacuo. Column chromatography (silica gel, 98:2 dichloromethane/methanol) afforded benzyl N-benzyloxycarbonyl)-3-aza-7-hydroxy-6-oxabicyclo[4.3.0]nonane-2-carboxylate (3.1 g, 62%) as a colorless oil: ¹H NMR (300 MHz, CDCl₃) δ 7.18-7.36 (m, 10H), 4.92-5.30 (m, 5H), 4.67-4.74 and 4.43-4.49 (2m, 1H), 4.25-4.38 (m, 1H), 4.17 and 3.59-3.87 (t and m, J=3.4 Hz, 3H), 2.75-2.85 and 2.44-2.51 (2m, 1H), 1.49-2.32 (m, 4H); MS (ESI) m/z 412 [C₂₃H₂₅NO₆+H]⁺.

Step (6): Preparation of benzyl N-(benzyloxycarbonyl)-3-aza-6-oxabicyclo[4.3.0]nonane-2-carboxylate

To a −78° C. solution of benzyl N-(benzyloxycarbonyl)-3-aza-7-hydroxy-6-oxabicyclo[4.3.0]nonane-2-carboxylate (3.1 g, 7.6 mmol) and triethylsilane (1.4 g, 11.7 mmol) in dichloromethane (150 mL) was added boron trifluoride etherate (1.2 g, 8.4 mmol) dropwise over 5 minutes. The solution was allowed to warm to 0° C. After 3 hours, TLC indicated that most of the starting material was consumed. A saturated sodium bicarbonate solution (3 mL) was added, and the mixture was stirred as it warmed to room temperature. Water (50 mL) was added, the layers were separated, and the organic layer was washed with a saturated sodium bicarbonate solution (50 mL) and brine (50 mL), dried over sodium sulfate, filtered, and concentrated in vacuo. Column chromatography (silica gel, 67:33 hexanes/ethyl acetate) afforded benzyl N-(benzyloxycarbonyl)-3-aza-6-oxabicyclo[4.3.0]nonane-2-carboxylate (2.1 g, 71%) as a colorless oil: ¹H NMR (300 MHz, CDCl₃) δ 7.18-7.36 (m, 10H), 5.22 and 5.14 (AB q, s, J=12.4 Hz, 2H), 4.92-5.08 (m, 2H), 4.37 and 4.30 (2 d, J=10.2, 10.1 Hz, 1H), 3.90-3.97 (m, 2H), 3.56-3.77 (m, 3H), 3.30-3.41 (m, 1H), 2.23-2.29 (m, 1H), 1.64-1.96 (m, 2H), 1.40-1.50 (m, 1H); MS (ESI) m/z 396 [C₂₃H₂₅NO₅+H]⁺.

Step (7): Synthesis of 3-aza-6-oxabicyclo[4.3.0]nonane-2-carboxylic Acid

To a solution of benzyl N-(benzyloxycarbonyl)-3-aza-6-oxabicyclo[4.3.0]nonane-2-carboxylate (2.1 g, 5.4 mmol) in ethanol (150 mL) and water (14 mL) was added palladium on carbon (10%, wet, 0.58 g). After hydrogenation for 2 hours at 35 pounds per square inch (“psi”), TLC analysis indicated that the reaction was complete. The mixture was filtered through celite, and the filter cake was rinsed with ethanol (3×20 mL). The filtrate was concentrated in vacuo, and the residue was redissolved in water, and refiltered through celite. Concentration of the filtrate afforded 3-aza-6-oxabicyclo[4.3.0]nonane-2-carboxylic acid (0.8 g, 86%) as a white solid: mp 239-242° C. (dec); ¹H NMR (300 MHz, D₂O) δ 4.14 (t, J=3.1 Hz, 1H), 3.96 (d, J=10.9 Hz, 1H), 3.81-3.87 (m, 1H), 3.20-3.42 (m, 1H), 3.35 (dd, J=12.9, 3.1 Hz, 1H), 3.20 (d, J=12.9 Hz, 1H), 2.21-2.28 (m, 1H), 1.93-1.99 (m, 1H), 1.79-1.91 (m, 1H), 1.58-1.74 (m, 1H), 1.36-1.43 (m, 1H); ¹³C NMR (75 MHz, D₂O) δ 174.67, 77.24, 67.49, 60.86, 51.81, 42.49, 21.46, 19.38; MS (ESI) m/z 172 [C₈H₁₃NO₃+H]⁺. Anal. Calcd. for C₈H₁₃NO₃: C, 56.13; H, 7.65; N, 8.18. Found: C, 55.89; H, 7.87; N, 7.90.

### COMPOUND EXAMPLE Q2

### Synthesis of 3-Aza-6,6-difluorobicyclo[3.3.0]octane-2-carboxylic acid

Step (1): Preparation of ethyl N-(Benzyloxycarbonyl)₃-aza-6,6-difluorobicyclo[3.3.0]octane-2-carboxylate

To a stirred solution of ethyl N-(benzyloxycarbonyl)₃-aza-6-oxobicyclo[3.3.0] octane-2-carboxylate (prepared according to the procedure described below in Compound Example Q6, 5.0 g, 15.1 mmol) in dichloromethane (125 mL) was added [bis(methoxyethyl)amino]sulfur trifluoride (5.7 g, 25.8 mmol) dropwise over 10 minutes. After the solution had stirred at room temperature for 72 hours, TLC analysis indicated that the starting material was nearly consumed. Saturated sodium bicarbonate (10 mL) and solid sodium bicarbonate were added until no more off-gassing was observed. The layers were separated, and the aqueous layer was extracted with dichloromethane (2×50 mL). The organic layers were combined, washed with saturated sodium bicarbonate (50 mL) and brine (50 mL), dried over sodium sulfate, filtered and concentrated in vacuo. Chromatography of the residue (silica gel, 85:15 to 70:30 hexanes/ethyl acetate) afforded ethyl N-(benzyloxycarbonyl)₃-aza-6,6-difluorobicyclo[3.3.0]octane-2-carboxylate (3.54 g, 66%) as an oil: ¹H NMR (300 MHz, CDCl₃) δ 7.31-7.36 (m, 5H), 5.16 and 5.12 (s, AB q, J=12.5 Hz, 2H), 4.03-4.29 (2 m, 3H), 3.80-3.90 (m, 1H), 3.64-3.76 (m, 1H), 2.85-2.95 (m, 2H), 2.05-2.27 (m, 3H), 1.70-1.82 (m, 1H), 1.27 and 1.16 (2t, J=7.1, 7.1 Hz, 3H); MS (ESI) m/z 354 [C₁₈H₂₁F₂NO₄+H]⁺. The oil was contaminated with about 10% impurity, assigned by mass spectral data as the corresponding vinyl mono fluoride.

Step (2): Preparation of N-(benzyloxycarbonyl)-3-aza-6,6-difluoro[3.3.0]octane-2-carboxylic Acid

A solution of ethyl N-(benzyloxycarbonyl)-3-aza-6,6-difluorobicyclo[3.3.0]octane-2-carboxylate (2.0 g, 5.7 mmol) and lithium hydroxide monohydrate (0.56 g, 13.3 mmol) in THF (35 mL) and water (17 mL) was stirred at room temperature. After 18 hours, TLC analysis indicated that the starting material was consumed. Ether (20 mL) was added, and the layers were separated. The aqueous layer was extracted with ether (2×10 mL), and acidified to pH 4 with 1 M HCl. The product was extracted into ether (3×20 mL). The combined organic extracts were washed with brine (20 mL), dried over sodium sulfate, filtered and concentrated in vacuo to afford N-(benzyloxycarbonyl)₃-aza-6,6-difluorobicyclo[3.3.0]octane-2-carboxylic acid as a crystallizing oil. To a solution of the oil (1.8 g, 5.1 mmol) in acetone (15 mL) and water (1 mL) cooled to 0° C. was added N-methyl morpholine-N-oxide (0.14 g, 1.2 mmol) and potassium osmate (0.01 g, 0.03 mmol). The solution was allowed to warm to room temperature and stir overnight. After cooling to 0° C., a 3% solution of sodium bisulfite (50 mL) was added. The mixture was allowed to warm to room temperature. Ethyl acetate (50 mL) was added, and the layers were separated. The aqueous layer was extracted with ethyl acetate (50 mL), and the organic layers were combined, dried over sodium sulfate, filtered and concentrated in vacuo. Chromatography (silica gel, dichloromethane to 90:10 dichloromethane/methanol) afforded ethyl N-(benzyloxycarbonyl)₃-aza-6,6-difluoro[3.3.0]octane-2-carboxylic acid (1.45 g, 79%) as a white solid: ¹H NMR (300 MHz, CDCl₃) δ 7.31-7.61 (m, 5H), 5.13-5.19 (s and m, 2H), 4.26-4.32 (m, 1H), 3.88 (dd, J=11.4, 4.6 Hz, 1H), 3.62-3.75 (m, 1H), 2.85-3.21 (m, 2H), 2.11-2.29 (m, 3H), 1.68-1.85 (m, 1H); MS (ESI) m/z 326 [C₁₆H₁₇F₂NO₄+H]⁺.

Step (3): Synthesis of 3-aza-6,6-difluorobicyclo[3.3.0]octane-2-carboxylic Acid

To a solution of N-(benzyloxycarbonyl)-3-aza-6,6-difluorobicyclo[3.3.0]octane-2-carboxylic acid (1.45 g, 4.7 mmol) in THF (120 mL) and water (12 mL) was added palladium on carbon (10%, wet, 0.48 g). The suspension was hydrogenated at 35 psi for 2.5 hours, at which time TLC analysis indicated that the starting material was consumed. The mixture was filtered through celite, and the filter cake was rinsed with THF (3×20 mL) and water (3×20 mL). The filtrate was concentrated in vacuo, and the residue was redissolved in water and filtered through celite. Concentration in vacuo afforded 3-aza-6,6-difluorobicyclo[3.3.0]octane-2-carboxylic acid (0.61 g, 78%) as a white solid: mp 200-205° C. (dec); ¹H NMR (300 MHz, CD₃OD) δ 3.68 (d, J=5.7, 1H), 3.49 (dd, J=12.6, 8.8 Hz, 1H), 3.34 (dd, J=12.8, 5.1 Hz, 1H), 3.03-3.18 (m, 2H), 2.12-2.33 (m, 3H), 1.84-1.93 (m, 1H); ¹³C NMR (75 MHz, CD₃OD) δ 174.24, 132.52 (t, J=252 Hz), 68.88, 47.16, 46.00, 35.11 (t, J=24.7 Hz), 28.04; ¹⁹F NMR (282 MHz, CD₃OD) δ-97.28 (d, J=234 Hz), −109.15 (d, J=234 Hz); MS (ESI) m/z 192 [C₈H₁₁F₂NO₂+H]⁺. Anal. Calcd. for C₈H₁₁F₂NO₂.0.4H₂O: C, 48.44; H, 6.00; N, 7.06. Found: C, 48.56; H, 5.74; N, 6.94.

### COMPOUND EXAMPLE Q3

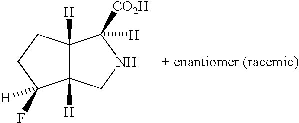

### Synthesis of a Racemic Mixture of (1S,2S,5S,6S)-3-aza-6-fluorobicyclo[3.3.0]octane-2-carboxylic Acid and (1R,2R,5R,6R)-3-aza-6-fluorobicyclo[3.3.0]octane-2-carboxylic Acid

Step (1): Preparation of ethyl N-(Benzyloxycarbonyl)-3-aza-6-fluorobicyclo[3.3.0]octane-2-carboxylate

To a stirred solution of diethylaminosulfur trifluoride (3.01 g, 18.7 mmol) in dichloromethane (400 mL) at −5° C. was added a solution of ethyl N-(benzyloxycarbonyl)-3-aza-6-hydroxybicyclo[3.3.0]octane-2-carboxylate (prepared as described below for Compound Example Q5, 5.9 g, 17.7 mmol) in dichloromethane (100 mL) dropwise over 30 minutes. After stirring for 30 minutes at 0° C., the solution was transferred to a separatory funnel and washed with water (75 mL), saturated sodium bicarbonate (75 mL) and brine (75 mL). The organic layer was dried over sodium sulfate, filtered and concentrated in vacuo. Chromatography (silica gel, 85:15 hexanes/ethyl acetate) afforded a 60:40 mixture of ethyl N-(benzyloxycarbonyl)₃-aza-6-fluorobicyclo[3.3.0]octane-2-carboxylate (MS (ESI) m/z 336 [C₁₈H₂₂FNO₄+H]⁺) and N-(benzyloxycarbonyl)-3-azabicyclo[3.3.0]octene-2-carboxylate (4.47 g) (MS (ESI) m/z 316 [C₁₈H₂₁NO₄+H]⁺) as a yellow oil. To a solution of this mixture (4.47 g) in acetone (85 mL) and water (15 mL) at 0° C. was added N-methyl morpholine-N-oxide (0.87 g, 7.41 mmol) and potassium osmate (0.008 g, 0.02 mmol). The mixture was allowed to warm to room temperature and stir overnight. The solution was cooled to 0° C., and a 3% solution of sodium bisulfite (50 mL) was added. After warming to room temperature, ethyl acetate (50 mL) was added, and the layers were separated. The aqueous layer was extracted with ethyl acetate (50 mL), and the organic layers were combined, dried over sodium sulfate, filtered and concentrated in vacuo. Chromatography (silica gel, dichloromethane to 93:7 dichloromethane/methanol) afforded pure ethyl N-(benzyloxycarbonyl)-3-aza-6-fluorobicyclo[3.3.0]octane-2-carboxylate (2.94 g, 50%) as a yellow oil: ¹H NMR (300 MHz, CDCl₃) δ 7.28-7.35 (m, 5H), 5.15 and 5.11 (s, AB q, J=12.4 Hz, 2H), 4.96-5.01 and 4.78-4.85 (2m, 1H), 4.03-4.26 (m, 3H), 3.82 (t, J=10.3 Hz, 1H), 3.26-3.38 (m, 1H), 2.83-3.01 (m, 2H), 2.19-2.32 (m, 1H), 1.60-2.05 (3m, 3H), 1.27 and 1.17 (2t, J=7.1, 7.1 Hz, 3H); MS (ESI) m/z 336 [C₁₈H₂₂FNO₄+H]⁺; and ethyl N-(benzyloxycarbonyl)-3-aza-6,7-dihydroxybicyclo[3.3.0]octane-2-carboxylate: ¹H NMR (300 MHz, CDCl₃) δ 7.30-7.36 (m, 5H), 5.00-5.35 (m, 2H), 4.04-4.32 (m, 5H), 3.72-3.91 (2 m, 1H), 3.48-3.65 (m, 1H), 2.95-3.04 and 2.56-2.71 (2 m, 2H), 2.19-2.46 (m, 2H), 1.62-1.85 (2 m, 2H), 1.13-1.33 (m, 3H); (ESI) m/z 350 [C₁₈H₂₃NO₆+H]⁺.

Step (2): Synthesis of a Racemic Mixture of (1S,2S,5S,6S)-3aza-6-fluorobicyclo[3.3.0]octane-2-carboxylic acid and (1R,2R,5R,6R)-3-aza-6-fluorobicyclo[3.3.0]octane-2-carboxylic Acid

In a manner similar to that described above for Compound Example Q2, ethyl N-(benzyloxycarbonyl)-3-aza-fluorobicyclo[3.3.0]octane-2-carboxylate was converted to the title compound: white solid; mp 197-225° C. (dec); ¹H NMR (300 MHz, D₂O) δ 4.94 (dd, J=7.8, 2.9 Hz, 1H), 3.56-3.67 (m, 2H), 2.93-3.10 (m, 2H), 2.77-2.91 (m, 1H), 1.73-2.14 (2m, 4H); ¹³C NMR (75 MHz, D₂O) δ 174.02, 99.49 (d, J=171 Hz), 49.39, 49.06, 47.20, 47.02, 45.92, 30.68 (d, J=21 Hz), 28.49; ¹⁹F NMR (282 MHz, D₂O) 8-173.20; MS (ESI) t/z 174 [C₈H₁₂FNO₂+H]⁺. Anal. Calcd. for C₈H₁₂FNO₂.0.4H₂O: C, 53.27; H, 7.15; N, 7.76; F, 10.53. Found: C, 53.49; H, 6.99; N, 7.61; F, 10.21; relative stereochemistry was determined by Nuclear Overhauser Effect nuclear magnetic resonance experiments.

### COMPOUND EXAMPLE Q4

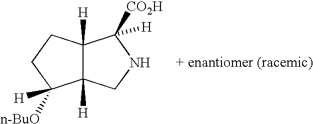

### Synthesis of a Racemic Mixture of (1S,2S,5R,6R)-3-aza-6n-butoxybicyclo[3.3.0]octane-2-carboxylic acid and (1R,2R,5S,6S)-3-aza-6n-butoxybicyclo[3.3.0]octane-2-carboxylic Acid

Step (1): Preparation of ethyl N-(Benzyloxycarbonyl)-3-aza-6n-butoxybicyclo[3.3.0]octane-2-carboxylate

To a stirred solution of ethyl N-(benzyloxycarbonyl)-3-aza-6-hydroxybicyclo[3.3.0]octane-2-carboxylate (prepared as described below for Compound Example Q5, 4.1 g, 12.3 mmol) and 1-iodobutane (22.2 g, 120 mmol) in DMF (75 mL) cooled to 0° C. was added sodium hydride (60% in mineral oil, 485 mg, 12.1 mmol) in small portions over 15 minutes. After stirring for 1.5 hours, a 0.5 M KHSO₄ solution (15 mL) was added in portions, and the mixture was stirred for 5 minutes. The solvent was removed in vacuo, and the residue was partitioned between ether and water. The aqueous layer was extracted with ether (3×100 mL), and the combined organic extracts were dried over sodium sulfate, filtered and concentrated in vacuo. Chromatography (silica gel, 85:15 hexanes/ethyl acetate) afforded ethyl N-(benzyloxycarbonyl)-3-aza-6n-butoxybicyclo[3.3.0]octane-2-carboxylate as two separate C6 epimers as colorless oils; epimer 1: (0.92 g, 19%); ¹H NMR (300 MHz, CDCl₃) δ 7.26-7.61 (m, 5H), 5.15 and 5.11 (s, AB q, J=12.4 Hz, 2H), 4.00-4.22 (2 m, 3H), 3.74-3.88 (m, 2H), 3.58 (td, J=14.2, 9.2 Hz, 1H), 3.27-3.43 (m, 2H), 2.74-2.86 (m, 1H), 2.68-2.72 (m, 1H), 1.59-1.98 (m, 4H), 1.46-1.55 (m, 2H), 1.12-1.37 (m, 2 t, J=7.1 Hz, 5H), 0.89 and 0.88 (2 t, J=7.2 Hz and J=7.3 Hz, 3H); MS (ESI) nz/z 390 [C₂₂H₃₁NO₅+H]⁺; epimer 2: (0.25 g, 5%) ¹H NMR (300 MHz, CDCl₃) δ 7.28-7.37 (m, 5H), 5.14 and 5.10 (s, AB q, J=12.5 Hz, 2H), 4.01-4.23 (2 m, 3H), 3.81 (td, J=10.1, 1.9 Hz, 1H), 3.64-3.71 (m, 1H), 3.32-3.44 (m, 3H), 2.76-2.84 (m, 1H), 2.68-2.77 (m, 1H), 2.11-2.20 (m, 1H), 1.86-1.98 (m, 1H), 1.64-1.75 (m, 1H), 1.47-1.61 (m, 3H), 1.34 (sext, J=7.4 Hz, 2H), 1.26 and 1.15 (2 t, J=7.1 Hz, 3H), 0.90 (t, J=7.2 Hz, 3H); MS (ESI) m/z 390 [C₂₂H₃₁NO₅+H]⁺.

Step (2): Synthesis of a Racemic Mixture of (1S,2S,5R,6R)-3-aza-6n-butoxybicyclo[3.3.0]octane-2-carboxylic acid and (1R,2R,5S,6S)-3-aza-6-n-butoxybicyclo[3.3.0]octane-2-carboxylic Acid

In a manner similar to that described above for Compound Example Q2, epimer 1 was converted to the title compound, which was obtained as a white solid: mp 180-184° C. (dec); ¹H NMR (300 MHz, D₂O) δ 3.91 (dt, J=7.1, 5.6 Hz, 1H), 3.63 (d, J=7.0 Hz, 1H), 3.26-3.46 (m, 3H), 3.21 (dd, J=12.1, 7.2 Hz, 1H), 2.96 (tt, J=8.7, 7.3 Hz, 1H), 2.76 (qd, J=8.2, 2.6 Hz, 1H), 1.56-1.90 (2m, 4H), 1.36-1.55 (m, 2H), 1.16-1.35 (m, 2H), 0.77 (t, J=7.3 Hz, 3H); ¹³C NMR (75 MHz, D₂O) δ 174.33, 81.09, 70.03, 68.37, 45.75, 45.36, 44.07, 31.40, 29.39, 27.59, 19.14, 13.49; MS (ESI) m/z 228 [C₁₂H₂₁NO₃+H]⁺; Anal. Calcd. for C₁₂H₂₁NO₃: C, 63.41; H, 9.31; N, 6.16. Found: C, 63.21; H, 9.36; N, 6.08; relative stereochemistry was determined by Nuclear Overhauser Effect nuclear magnetic resonance experiments.

### COMPOUND EXAMPLE Q5

### Synthesis of 3-aza-6-hydroxybicyclo[3.3.0]octane-2-carboxylic Acid Hydrochloride

Step (1): Preparation of Ethyl N-(Benzyloxycarbonyl)-3-aza-6-hydroxybicyclo[3.3.0]octane-2-carboxylate

To a solution of ethyl N-(benzyloxycarbonyl)₃-aza-6-oxobicyclo[3.3.0]octane-2-carboxylate (prepared in a manner similar to that described above for the preparation of the intermediate of Compound Example Q1, Step 3, 8.7 g, 26.3 mmol) in THF (157 mL) and water (7 mL) at 0° C. was added sodium borohydride (689 mg, 18.2 mmol). The rate of addition was adjusted to maintain a reaction temperature of 5-10° C. After stirring at 0-5° C. for 2 hours past complete addition, TLC analysis indicated that the starting material was consumed. Saturated ammonium chloride (100 mL) was added over 30 minutes at a rate to maintain the temperature at 5-10° C. After an additional 30 minutes, the layers were separated, and the aqueous layer was extracted with ethyl acetate (2×100 mL). The organic layers were combined, dried over sodium sulfate, filtered and concentrated in vacuo. The residue was chromatographed (silica gel, 50:50 hexanes/EtOAc) to afford ethyl N-(benzyloxycarbonyl)₃-aza-6-hydroxybicyclo[3.3.0]octane-2-carboxylate (3:1 ratio of C6 epimers, 7.6 g, 87%) as a colorless oil: ¹H NMR (300 MHz, CDCl₃) δ 7.28-7.37 (m, 5H), 5.16, 5.11 (s and AB q, J=12.4 Hz, 2H), 3.97-4.29 (m, 4H), 3.86-3.95 (m, 0.75H), 3.74-3.82 (m, 0.25H), 3.56-3.65 (m, 0.75H), 3.32-3.43 (m, 0.25H), 2.84-2.90 (m, 0.25H), 2.59-2.81 (m, 2.75H), 2.21-2.31 (m, 0.25H), 1.52-2.03 (m, 3.75H), 1.26 and 1.14 (2t, J=7.1 Hz, 3H).

Step (2): Synthesis of 3-aza-6-hydroxybicyclo[3.3.0]octane-2-carboxylic Acid Hydrochloride

In a manner similar to that described above for Compound Example Q2, ethyl N-(benzyloxycarbonyl)₃-aza-6-hydroxybicyclo[3.3.0]octane-2-carboxylate was converted to 3-aza-6-hydroxybicyclo[3.3.0]octane-2-carboxylic acid hydrochloride (3:1 ratio of C6 epimers, 0.61 g, 77%) as a white solid: mp (DSC) 152.4166.3° C. (endotherm), 194.6-200.5° C. (endotherm), 241.5-268.2 (endotherm), 347.4-357.1 (endotherm); ¹H NMR (300 MHz, D₂O) δ 4.18 (q, J=5.3 Hz, 0.75H), 4.03-4.05 (m, 0.25H), 3.89 (d, J=6.3 Hz, 0.75H), 3.82 (d, J=7.6 Hz, 0.25H), 3.62 (dd, J=12.1, 9.0 Hz, 0.25H), 3.36 (d, J=6.6 Hz, 1.5H), 2.82-3.04 (m, 2H), 2.67-2.76 (m, 0.25H), 1.63-2.17 (m, 4H); ¹³C NMR (75 MHz, D₂O) δ 171.94, 171.80, 76.58, 72.93, 66.33, 65.29, 50.50, 48.50, 46.63, 45.78, 45.51, 33.30, 32.48, 28.34, 27.94; IR (ATR) 3362, 2946, 2776, 1743, 1553, 1403, 1192, 959, 816 cm⁻¹; MS (ESI) m/z 172 [C₈H₁₃NO₃+H]⁺. Anal. Calcd. for C₈H₁₃NO₃.HCl.0.2H₂O.0.4NH₄Cl: C, 41.30; H, 6.93; Cl, 21.33; N, 8.43. Found: C, 41.03; H, 6.66; Cl, 21.66; N, 8.24.

### COMPOUND EXAMPLE Q6

### Synthesis of 3-aza-6-oxobicyclo[3.3.0]octane-2-carboxylic Acid Hydrochloride

In a manner similar to that described above for Compound Example Q2, ethyl N-(benzyloxycarbonyl)₃-aza-6-oxobicyclo[3.3.0]octane-2-carboxylate (prepared in a manner similar to that described above for the preparation of the intermediate of Compound Example Q1, Step (3)) was converted to 3-aza-6-oxobicyclo[3.3.0]octane-2-carboxylic acid hydrochloride (0.6 g, 64%): DSC 108.3-111.2° C. (endotherm), 331.6-342.6° C. (endotherm); ¹H NMR (300 MHz, D₂O) δ 4.09 (d, J=7.8 Hz, 1H), 3.61 (dd, J=12.4, 9.5 Hz, 1H), 3.43 (dd, J=12.4, 4.1 Hz, 1H), 3.25-3.34 (m, 1H), 3.17 (td, J=9.3, 4.1 Hz, 1H), 2.42-2.49 (m, 2H), 2.25-2.38 (m, 1H), 2.02-2.13 (m, 1H); ¹³C NMR (75 MHz, D₂O) δ 222.18, 171.29, 64.32, 50.89, 46.64, 44.56, 36.44, 23.73; IR (ATR) 3485, 3400, 2999, 2758, 2517, 1732, 1606, 1574, 1431, 1390, 1224, 872 cm⁻¹; MS (ESI) m/z 170 [C₈H₁₁NO₃+H]⁺. Anal. Calcd. for C₈H₁₁NO₃—HCl-1.25H₂O: C, 42.11; H, 6.41; Cl, 15.54; N, 6.14. Found: C, 42.32; H, 6.28; Cl, 15.75; N, 5.95.

Alternatively to Scheme O, compounds of Formula I wherein any two groups as identified above for Formula I are taken together with the ring atoms of Formula I to which they are attached to form a bridged ring may be prepared by conventional means as illustrated below in Scheme R.

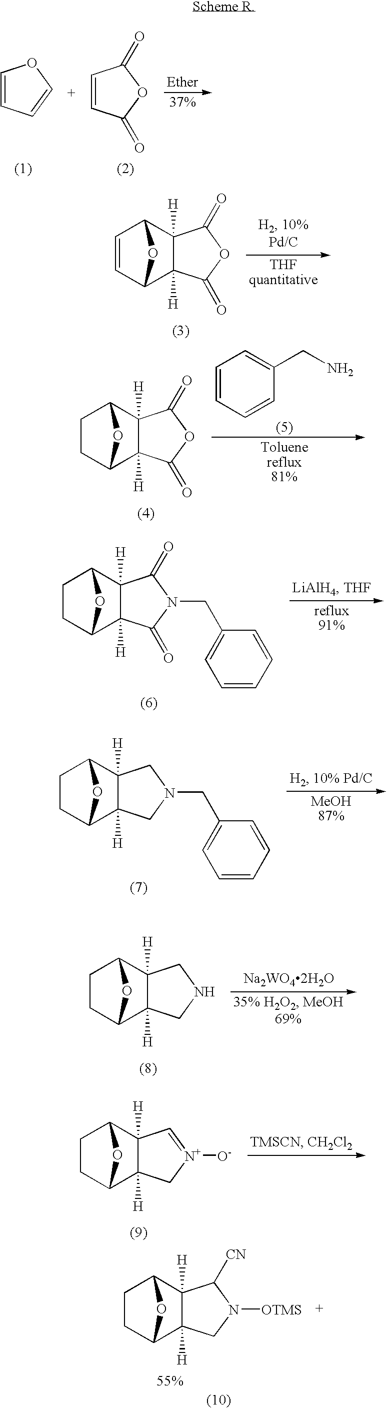

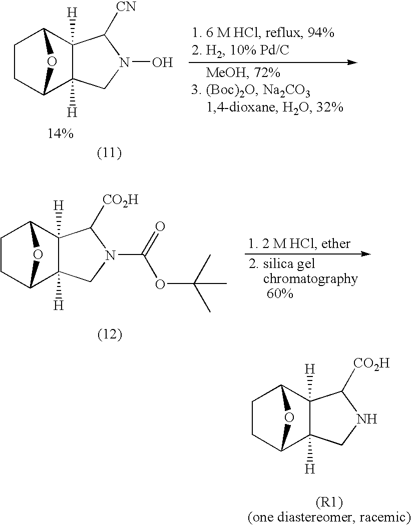

Compounds of Formula I were prepared according to the method illustrated in Scheme R as described below for Compound Examples R1 to R3.

### COMPOUND EXAMPLE R1

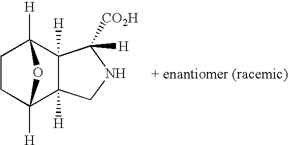

### Synthesis of a Racemic Mixture of (1R,2R,3R,6S,7S)-10-oxa-4aza-tricyclo[5.2.1.0^{2,6}]decane-3-carboxylic Acid and (1S,2S,3S,6R,7R)-10-oxa-4-aza-tricyclo[5.2.1.0^{2,6}]decane-3-carboxylic Acid

Step (1): Preparation of 4-hydroxy-10-oxa-4-azonia-tricyclo[5.2.1.0^{2,6}]dec-3-ene

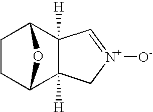

To a solution of 10-oxa-4-aza-tricyclo[5.2.1.0^{2,6}]decane (Beilstein Registry Number 1209516 and CAS Registry Number 73611-42-4, 4.0 g, 28.8 mmol) in methanol (60 mL) at 0° C. was added Na₂WO₄-2H₂O (0.47 g, 1.44 mmol) followed by a 35% aqueous hydrogen peroxide solution (6.0 mL, 64.7 mmol) added dropwise over a 15 minute period. The reaction mixture was stirred at 0° C. for 6 hours and diluted with water (50 mL) and dichloromethane (150 mL). The organic layer was separated and the aqueous phase was extracted with dichloromethane (3×150 mL). The combined organic layers were dried over sodium sulfate, filtered and concentrated in vacuo to give a yellow oil. Silica gel chromatography (eluant 9:1 dichloromethane/methanol) afforded the title compound (3.0 g, 69%) as a low melting point solid: ¹H NMR (300 MHz, CDCl₃) δ 1.53-1.58 (m, 2H), 1.75-1.79 (m, 2H), 2.72-2.76 (m, 1H), 3.13-3.16 (m, 1H), 3.78-3.83 (m, 1H), 4.064.09 (m, 1H), 4.44 (d, J=4.7 Hz, 1H), 4.50 (d, J=4.7 Hz, 1H), 6.82 (d, J=1.9 Hz, 1H); ¹³C NMR (75 MHz, CDCl₃) δ 27.6, 28.3, 41.5, 52.3, 66.6, 77.7, 80.8, 135.0; MS (ESI) m/z 154 [M+H]⁺.

Step (2): Preparation of 4-trimethylsilanyloxy-10-oxa-4-aza-tricyclo[5.2.1.0^{2,6}]decane-3-carbonitrile and 4-hydroxy-10-oxa-4-aza-tricyclo[5.2.1.0^{2,6}]decane-3-carbonitrile

To a solution of the product of Step (1) (3.0 g, 19.6 mmol) in dichloromethane (40 mL) was added trimethylsilyl cyanide (7.7 mL, 57.6 mmol). The reaction mixture was stirred at room temperature overnight and diluted with water (50 mL) and dichloromethane (100 mL). The organic layer was separated and the aqueous phase was extracted with dichloromethane (3×100 mL). The combined organic layers were dried over sodium sulfate, filtered and concentrated in vacuo to give a mixture of two title products. These products were separated by silica gel chromatography (eluant 9:1 hexanes/ethyl acetate) to give 4-trimethylsilanyloxy-10-oxa aza-tricyclo[5.2.1.0^{2,6}]decane-3-carbonitrile (2.7 g, 55%) as a low melting point solid: ¹H NMR (300 MHz, CDCl₃) δ-0.04 (s, 9H), 1.20-1.24 (m, 2H), 1.50-1.58 (m, 2H), 2.10-2.28 (m, 2H), 2.50-2.70 (m, 1H), 3.05-3.18 (m, 2H), 4.04-4.05 (m, 1H), 4.20-4.21 (m, 1H); MS (ESI) m/z 253 [M+H]⁺; and 4-hydroxy-10-oxa-4aza-tricyclo[5.2.1.0^{2,6}]decane-3-carbonitrile (0.5 g, 14%) as a white solid: ¹H NMR (300 MHz, CDCl₃) δ 1.45-1.51 (m, 2H), 1.71-1.83 (m, 2H), 2.62-2.63 (m, 3H), 3.50 (dd, J=10.1, 7.8 Hz, 2H), 4.31 (d, J=4.7 Hz, 1H), 4.45 (d, J=4.9 Hz, 1H), 7.14 (br s, 1H); MS (ESI) m/z 181 [M+H]⁺.

Step (3): Preparation of 10-Oxa-4-aza-tricyclo[5.2.1.0^{2,6}]decane-3-carboxylic Acid 4-tert-butyl Ester

A mixture of 4-trimethylsilanyloxy-10-oxa aza-tricyclo[5.2.1.0^{2,6}]decane-3-carbonitrile (2.7 g, 10.7 mmol) and 6 M HCl (100 mL) was heated to reflux for 6 hours. The reaction mixture was allowed to cool to room temperature and ether (40 mL) was added. The organic layer was separated and the aqueous phase was concentrated in vacuo to give the crude acid (1.5 g, 94%) as a foam: MS (ESI) m/z 200 [M+H]⁺. To a Parr bottle containing 10% Pd/C (0.4 g) was added methanol (80 mL) under an atmosphere of nitrogen. The mixture was shaken with hydrogen (40 psi) for 15 minutes to pre-reduce the catalyst. A solution of the above acid (1.5 g) in methanol (80 mL) was added to the pre-reduced catalyst and the reaction mixture shaken for 24 hours under hydrogen (50 psi). The mixture was filtered through a pad of celite, and the filtrate concentrated to give crude amino acid (1.0 g, 72%) as a colorless foam: MS (ESI) m/z 184 [M+H]⁺. To this amino acid (1.0 g, 5.46 mmol) in 1,4-dioxane (10 mL) was added a solution of sodium carbonate (2.3 g, 4.0 mmol) in water (50 mL). A solution of di-tert-butyldicarbonate (2.4 g, 10.9 mmol) in 1,4-dioxane (10 mL) was added to the reaction mixture and stirred at room temperature overnight. The reaction mixture was diluted with ethyl acetate (150 mL) and aqueous 1 M HCl (20 mL). The organic layer was separated and the aqueous phase was extracted with ethyl acetate (2×100 mL). The combined organic layers were washed with brine and dried over sodium sulfate. Solvent removal and purification of the residue by silica gel chromatography (eluant 9:1 dichloromethane/methanol) afforded the title compound (0.9 g, 32%) as a colorless foam: ¹H NMR (300 MHz, CDCl₃) δ 1.43 (s, 9H), 1.44-1.46 (m, 2H), 1.62-1.69 (m, 2H), 2.52-2.62 (m, 2H), 3.53-3.55 (m, 1H), 3.67-3.69 (m, 1H), 4.18-4.26 (m, 1H), 4.40-4.42 (m, 1H), 4.56 (d, J=4.1 Hz, 1H), 7.84 (br s, 1H); MS (ESI) m/z 284 [M+H]⁺.

Step (4): Synthesis of a Racemic Mixture of (1R,2R,3R,6S,7S)-10-oxa-4aza-tricyclo[5.2.1.0^{2,6}]decane-3-carboxylic Acid and (1S,2S,3S,6R,7R)-10-oxa-4-aza-tricyclo[5.2.1.0^{2,6}]decane-3-carboxylic acid

A solution of the product of Step (3) (0.9 g, 3.18 mmol) in 2 M HCl (10 ml, in ether) was stirred at room temperature overnight. The precipitate was collected and washed with ether and dried to give the product (0.65 g) as a hygroscopic solid. The product was further purified by silica gel chromatography (eluant 60:30:10 dichloromethane/methanol/ammonium hydroxide) to give the amino acid (0.35 g, 60%) as a pale yellow solid: mp 188-190° C.; ¹H NMR (300 MHz, CD₃OD) δ 1.52-1.55 (m, 2H), 1.66-1.70 (m, 2H), 2.64-2.69 (m, 2H), 3.03 (dd, J=12.1, 5.4 Hz, 1H), 3.52 (dd, J=12.1, 8.0 Hz, 1H), 3.73 (d, J=5.2 Hz, 1H), 4.42 (d, J=4.5 Hz, 1H), 4.58 (d, J=4.7 Hz, 1H); ¹³C NMR (75 MHz, CD₃OD) δ 28.8, 48.4, 50.6, 53.6, 67.9, 81.8, 82.2, 173.0; MS (ESI) m/z 184 [M+H]⁺; relative stereochemistry was determined by Nuclear Overhauser Effect nuclear magnetic resonance experiments.

### COMPOUND EXAMPLE R2

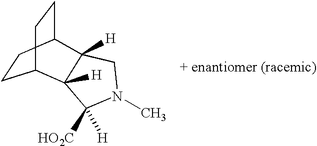

### Synthesis of a Racemic Mixture of (2R,3R,6S)-4-methyl-4-aza-tricyclo[5.2.2.0^{2,6}]undecane-3-carboxylic Acid Hydrochloride and (2S,3S,6R)-4-methyl-4-aza-tricyclo[5.2.2.0^{2,6}]undecane-3-carboxylic Acid Hydrochloride

To a suspension of the compound of Compound Example R3 below (1.6 g, 6.91 mmol) in water (16 mL), was added sodium carbonate (0.75 g, 6.91 mmol) followed by formalin (2.5 mL, 37% solution, 34.6 mmol). After stirring for 10 minutes, the mixture was added dropwise to a stirred solution of sodium borohydride (0.65 g, 17.3 mmol) in tetrahydrofuran (30 mL). The reaction mixture was stirred overnight at room temperature and then concentrated to dryness. The crude residue was dissolved in methanol, reduced in volume to approximately 5 mL and applied directly to a silica gel column (eluant dichloromethane/methanol/ammonium hydroxide 90:10:2) to afford the free base of the title compound (0.8 g, 55%) as a white solid. This white solid was stirred in 2 M HCl (20 mL, ether) for 3 hours and the product was collected by filtration and dried to give the title compound (0.9 g): mp 262-263° C.; ¹H NMR (300 MHz, CD₃OD) δ 1.53-1.61 (m, 5H), 1.74-1.86 (m, 5H), 2.46-2.54 (m, 2H), 3.06 (s, 3H), 3.16 (t, J=9.9 Hz, 1H), 3.70-3.72 (m, 1H), 4.11 (d, J=9.7 Hz, 1H); ¹³C NMR (75 MHz, CD₃OD) δ 20.0, 20.2, 26.2, 26.3, 26.5, 26.6, 39.0, 39.8, 46.2, 58.9, 70.5, 170.4; MS (ESI) m/z 210 [M+H]⁺; relative stereochemistry was determined by Nuclear Overhauser Effect nuclear magnetic resonance experiments.

### COMPOUND EXAMPLE R3

### Synthesis of 4-aza-tricyclo[5.2.2.0^{2,6}]undecane-3-carboxylic Acid Hydrochloride

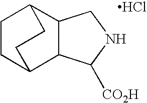 Step (1): Preparation of 4-aza-tricyclo[5.2.2.0^{2,6}]undec-3-ene 4-oxide

To a solution of 4-aza-tricyclo[5.2.2.0^{2,6}]undecane (Beilstein Registry Number 1634123; CAS Registry Number 4764-23-2, 2.9 g, 19.2 mmol) in methanol (30 mL) at 0° C. was added Na₂WO₄.2H₂O (0.26 g, 0.77 mmol) followed by 35% aqueous hydrogen peroxide solution (5.0 mL, 57.6 mmol) added dropwise addition over 15 minutes. The reaction mixture was stirred at 0° C. for 4 hours and diluted with water (20 mL) and dichloromethane (50 mL). The organic layer was separated, and the aqueous phase was extracted with dichloromethane (3×50 mL). The combined organic layers were dried over sodium sulfate, filtered and concentrated in vacuo to give a yellow oil. Silica gel chromatography using methylene chloride/methanol (9:1) as eluant afforded the title compound (2.5 g, 80%) as a white solid: ¹H NMR (300 MHz, CDCl₃) δ 1.1441.47 (m, 1H), 1.59-1.74 (m, 8H), 1.83 (s, 1H), 2.68-2.69 (m, 1H), 3.13 (d, J=10.4 Hz, 1H), 3.83-3.89 (m, 1H), 4.18 (dd, J=14.1, 11.8 Hz, 1H), 6.87 (d, J=1.2 Hz, 1H); MS (ESI) m/z 166 [M+H]⁺.

Step (2): Preparation of 4-hydroxy-4-aza-tricyclo[5.2.2.0^{2,6}]undecane-3-carbonitrile

To a solution of the compound of Step (1) (2.9 g, 17.6 mmol) in methanol (40 mL) was added potassium cyanide (2.2 g, 35.2 mmol) and 4 M HCl (7 mL). The reaction mixture was stirred at 0° C. for 4 hours and was quenched with 10% aqueous sodium hydrogen sulfite (10 mL). The aqueous phase was made alkaline to pH 10 with 2 M NaOH and extracted with dichloromethane (3×100 mL). The combined organic layers were dried over sodium sulfate and concentrated in vacuo. Purification of residue by silica gel chromatography using hexanes/ethyl acetate (1:1) as eluant afforded the desired nitrile 67 (2.0 g, 59%) as a thick oil: ¹H NMR (300 MHz, CDCl₃) δ 1.44-1.51 (m, 5H), 1.64-1.70 (m, 5H), 2.36-2.38 (m, 1H), 2.50-2.53 (m, 1H), 2.91-2.92 (m, 1H), 3.41 (t, J=8.3 Hz, 1H), 3.72-3.74 (m 1H), 7.02 (s, 1H); ¹³C NMR (75 MHz, CDCl₃) δ 19.6, 20.0, 25.0, 25.6, 25.7, 25.8, 36.5, 43.5, 59.6, 60.7, 119.1; MS (ES) nm/z 193 [M+H]⁺.

Step (3): 4-hydroxy-4-aza-tricyclo[5.2.2.0^{2,6}]undecane-3-carboxylic Acid

A solution of the product of Step (2) (1.0 g, 5.18 mmol) in concentrated hydrochloric acid (10 mL) was heated at 50° C. overnight. The reaction mixture was allowed to cool to room temperature and the solvent was removed in vacuo to afford the acid 68 (1.0 g, 91%) as a white solid: ¹H NMR (300 MHz, CD₃OD) δ 1.54-1.82 (m, 10H), 2.44-2.57 (m, 2H), 3.53 (t, J=10.7 Hz, 1H), 3.98 (dd, J=11.3, 7.9 Hz, 1H), 4.40 (d, J=10.6 Hz, 1H); MS (ESI) m/z 212 [M+H]⁺.

Step (4): Synthesis of 4-aza-tricyclo[5.2.2.0^{2,6}]undecane-3-carboxylic Acid Hydrochloride

To a Parr bottle containing 10% Pd/C (0.1 g) was added methanol (30 mL) under an atmosphere of nitrogen. The mixture was shaken with hydrogen (40 psi) for 20 minutes to pre-reduce the catalyst. A solution of the compound of Step (3) (1.0 g, 4.74 mmol) in methanol (30 mL) was added to the pre-reduced catalyst and the reaction mixture shaken for 3 hours under hydrogen (60 psi). The mixture was filtered through a pad of celite, and the filtrate concentrated to afford the crude amino acid (1.0 g, 99%) as a colorless oil: MS (ESI) m/z 196 [M+H]⁺. To a solution of the above amino acid (1.0 g, 5.1 mmol) in 1,4-dioxane (10 mL) was added a solution of sodium carbonate (0.54 g, 5.1 mmol) in water (10 mL). A solution of di-tert-butyldicarbonate (1.68 g, 7.7 mmol) in 1,4-dioxane (5 mL) was added to the reaction mixture and stirred at room temperature for 4 hours. The reaction mixture was diluted with ethyl acetate (50 mL) and aqueous 1 M HCl (10 mL). The organic layer was separated, and the aqueous phase was extracted with ethyl acetate (2×50 mL). The combined organic layers were washed with brine and dried over sodium sulfate. Solvent removal followed by purification of the residue by silica gel chromatography using hexanes/ethyl acetate (1:9) as eluant afforded the desired acid (1.2 g, 79%) as a colorless foam: ¹H NMR (300 MHz, CDCl₃) δ 1.44 (s, 9H), 1.47-1.63 (m, 10H), 2.37-2.50 (m, 2H), 3.51-3.71 (m, 2H), 4.22-4.37 (m, 1H), 11.3 (br s, 1H); MS (ESI) m/z 296 [M+H]⁺, 196 [M+H−100(Boc)]+. A solution of the acid (1.2 g, 4.1 mmol) in 2 M HCl (20 ml, in ether) was stirred at room temperature overnight. The precipitate was collected and washed with ether and dried to give the title compound (0.72 g, 77%) as a white solid: mp 260-265° C.; ¹H NMR (300 MHz, CD₃OD) δ 1.51-1.75 (m, 9H), 1.87-1.88 (m, 1H), 2.38-2.45 (m, 1H), 2.53-2.56 (m, 1H), 3.15 (t, J=9.9 Hz, 1H), 3.53 (dd, J=11.8, 8.4 Hz, 1H), 4.19 (d, J=9.9 Hz, 1H); ¹³C NMR (75 MHz, CD₃OD) δ 20.0, 20.3, 26.3, 26.5, 40.1, 44.9, 48.3, 62.9, 170.9; MS (ESI) m/z 196 [M+H]⁺. Anal. Calcd. for C₁₁H₁₇NO₂—HCl: C, 57.02; H, 7.83; N, 6.04; Cl, 15.30. Found: C, 56.70; H, 7.79; N, 6.00; Cl, 15.40.

Alternatively to Scheme R, compounds of Formula I wherein R¹⁰ and/or R^{10w} are not H in Formula I may be prepared by conventional means as illustrated below in Scheme S. 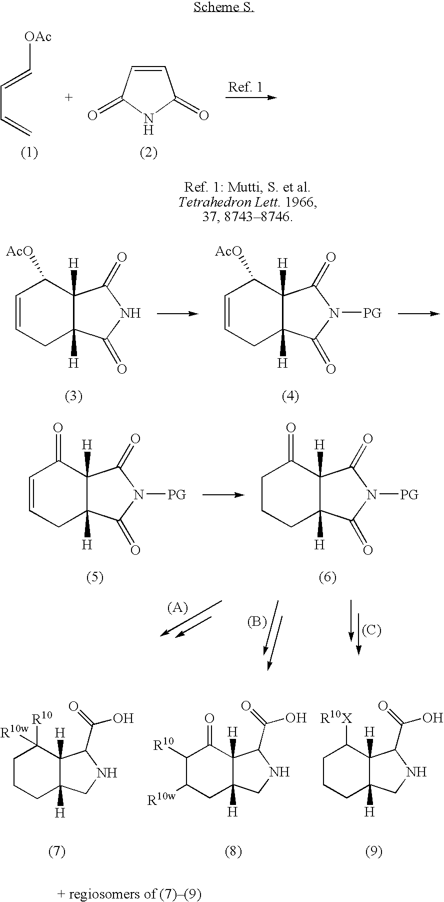
wherein (A) (i) via a Grignard or Suzuki/triflate (R¹⁰ aryl, R^{10w} is H) or (ii) fluorination (R¹⁰ and R^{10w} are each F); (B) via (i) alpha oxidation, (ii) elimination to 3,4-enone, and (iii) reductive alkylation (R¹⁰ is not H, R^{10w} is H) or (iii) 1,4-addition (R¹⁰ is H, R^{10w} is not H); (C) reductive amination (X is NH); reduction followed by (i) alkylation (X is O); or (ii) sulfanation (X is S) (ii) fluorination (R¹⁰ is F, R^{10w} is H), or (iii) coupling with R¹⁰SH.

Corresponding derivatives wherein the oxo intermediate is in the 5- or 6-position of Formula I may be prepared according to the methods described above for the conversion of the compound of formula (6) in Scheme S to the compounds of formulas (7)-(9) plus regioisomers thereof, wherein the oxo intermediate is prepared as illustrated below in Scheme T. 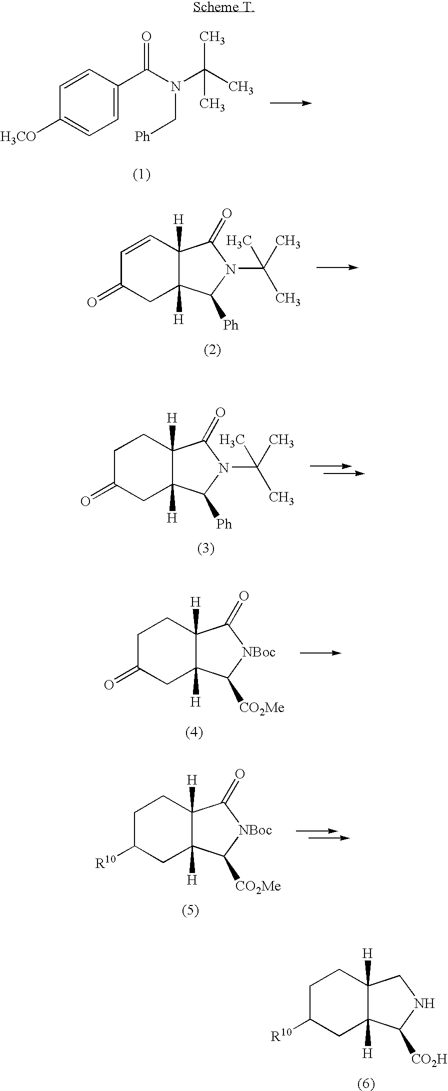
For the preparation of a compound of formula (4) in Scheme T, see (i) Clayden, J. et al., Chem. Commun., 1999; 231-232, (ii) Clayden, J. et al., Chem. Commun., 2000; 317-318, and (iii) Clayden, J. et al., Synlett, 2001; 302-304.

An artisan of ordinary skill will appreciate that the synthetic routes to the compounds of Formula I illustrated in the above schemes may be adapted for the preparation of invention compounds other than what is directly shown. For example, preparations of compounds of Formula I wherein Y⁴ to Y⁷ are carbon-based may be adapted to prepare compounds of Formula I wherein one or two non-adjacent Y⁴ to Y⁷ groups are O, S, S(O), S(O)₂, or NR⁵. Compounds of Formula I wherein one or two non-adjacent Y⁴ to Y⁷ groups are S(O) or S(O)₂ may be prepared by oxidation of the corresponding compound wherein the one or two non-adjacent Y⁴ to Y⁷ groups are S. The artisan will also appreciate that the above methods are not the only routes by which compounds of Formula I may be prepared. Further, the artisan will appreciate that the reagents used to illustrate the above methods are not the only reagents that may be used. For example, esters may be saponified under basic or acidic conditions and amides may be prepared by coupling a carboxylic acid with a primary or secondary amine using coupling agents such as dicyclohexylcarbodiimide (“DCC”), a water soluble carbodiimide, P(Ph)₃ and diethylazodicarboxylate, bis(2-oxo-3-oxazolidinyl)phosphinic chloride (BOP-Cl), POCl₃, Ti(Cl)₄, and others.

The ability of the invention compounds to inhibit joint cartilage damage, alleviate joint pain, and treat osteoarthritis has been established in animal models as described below.

### Biological Method 1

Monosodium Iodoacetate-Induced Osteoarthritis in Rat Model of Joint Cartilage Damage (“MIA Rat”):

One end result of the induction of osteoarthritis in this model, as determined by histologic analysis, is the development of an osteoarthritic condition within the affected joint, as characterized by the loss of Toluidine blue staining and formation of osteophytes. Associated with the histologic changes is a concentration-dependent degradation of joint cartilage, as evidenced by affects on hind-paw weight distribution of the limb containing the affected joint, the presence of increased amounts of proteoglycan or hydroxyproline in the joint upon biochemical analysis, or histopathological analysis of the osteoarthritic lesions.

The invention compounds typically are not effective for relieving joint pain when administered in an acute model, such as the instant MIA Rat model, which has a duration of just 14 or 28 days. The hind-paw weight distribution effects observed below, or the effects that would be expected to be observed, for an invention compound results from the invention compound's ability to directly inhibit damage to cartilage.

Generally, In the MIA Rat model on Day 0, the hind-paw weight differential between the right arthritic joint and the left healthy joint of male Wistar rats (150 g) are determined with an incapacitance tester, model 2KG (Linton Instrumentation, Norfolk, United Kingdom). The incapacitance tester has a chamber on top with an outwardly sloping front wall that supports a rat's front limbs, and two weight sensing pads, one for each hind paw, that facilitates this determination. Then the rats are anesthetized with isofluorine, and the right, hind leg knee joint is injected with 1.0 mg of mono-iodoacetate (“MIA”) through the infrapatellar ligament. Injection of MIA into the joint results in the inhibition of glycolysis and eventual death of surrounding chondrocytes. The rats are further administered either an invention compound or vehicle (in the instant case, water) daily for 14 days or 28 days.

The invention compound is typically administered at a dose of 30 mg of per kilogram of rat per day (30 mg/kg/day), but may be administered at other doses such as, for example, 10 mg/kg/day, 60 mg/kg/day, 90-mg/kg/day, or 100 mg/kg/day according to the requirements of the invention compound being studied. It is well within the level of ordinary skill in the pharmaceutical arts to determine a proper dosage of an invention compound in this model.

Administration of an invention compound in this model is optionally by oral administration or by intravenous administration via an osmotic pump. After 7 and 14 days for a two week study, or 7, 14, and 28 days for a four week study, the hind-paw weight distribution is again determined. Typically, the animals administered vehicle alone place greater weight on their unaffected left hind paw than on their right hind paw, while animals administered an invention compound show a more normal (i.e., more like a healthy animal) weight distribution between their hind paws. This change in weight distribution was proportional to the degree of joint cartilage damage. Percent inhibition of a change in hind paw joint function is calculated as the percent change in hind-paw weight distribution for treated animals versus control animals. For example, for a two week study,

$$\begin{matrix}\text{Percent inhibition of a change} \\ \text{in hind paw joint function}\end{matrix} = \{ 1 - [ \frac{( Δ {W}_{G} )}{( Δ {W}_{C} )} ] \} \times 100$$

wherein: ΔW_{C} is the hind-paw weight differential between the healthy left limb and the arthritic limb of the control animal administered vehicle alone, as measured on Day 14; and
ΔW_{G} is the hind-paw weight differential between the healthy left limb and the arthritic limb of the animal administered an invention compound as measured on Day 14.

In order to measure biochemical or histopathological end points in the MIA Rat model, some of the animals in the above study may be sacrificed, and the amounts of free proteoglycan in both the osteoarthritic right knee joint and the contralateral left knee joint may be determined by biochemical analysis. The amount of free proteoglycan in the contralateral left knee joint provides a baseline value for the amount of free proteoglycan in a healthy joint. The amount of proteoglycan in the osteoarthritic right knee joint in animals administered an invention compound and the amount of proteoglycan in the osteoarthritic right knee joint in animals administered vehicle alone, are independently compared to the amount of proteoglycan in the contralateral left knee joint. The amounts of proteoglycan lost in the osteoarthritic right knee joints are expressed as percent loss of proteoglycan compared to the contralateral left knee joint control. The percent inhibition of proteoglycan loss, may be calculated as {[(proteoglycan loss from joint (%) with vehicle)−(proteoglycan loss from joint with 1-substituted 1,3,3a,4,5,6,7,7a-octahydroisoindole-1-carboxylic acid)]÷(proteoglycan loss from joint (%) with vehicle)}×100.

### Biological Method 2

The Compound of Compound Example B1 in MIA:

Administration via osmotic pump is preferable for screening compounds. In a particular experiment, monosodium iodoacetate (“MIA”) (1 mg/joint) was injected through the infrapatellar ligament of the right knee of anesthetized male, Wistar rats. The contralateral control knee was injected with 50 μL of physiologic saline. The change in hind paw weight distribution, as determined by use of an incapacitance tester, between the right (arthritic) and left (contralateral control) knees was utilized as an index of functional limitation in the arthritic knee. Limitations in joint function were determined on days 7 and 14 following induction of arthritis. (Optionally, the animals could be sacrificed and erosion severity determined on the tibial plateaus from the arthritic joint. Histological analysis could also conducted on these samples.)

The basis of the invention is derived from the ability of the invention compounds, evidenced by, among others, the compound of Compound Example B1 to inhibit loss of joint function after administration subcutaneously via osmotic pumps at 10-mg/kg/day (dosing may also be carried out at, for example, 100-mg/kg/day, 90-mg/kg/day, or 30-mg/kg/day). The results of this and other studies with dosing by osmotic pump is shown below in Table 1 in columns labelled either “% I @ 10 mg/kg/day (%±SEM).” The phrase “% I @ 10 mg/kg/day (%) (±SEM)” means percent inhibition of loss of hind limb joint function at 10 milligrams per kilogram per day osmotic pump dosing, expressed as a percentage inhibition, optionally plus or minus a standard error measure.

TABLE 1
Inhibition of loss of hind limb joint function in MIA at Day 14

| Compound | % I @ 10 |
|---|---|
| Example | mg/kg/day |
| No. | (%) (± SEM) |
| B1 | 32 |
| B2 | −5 |
| B3 | n/a¹ |
| B4 | 13 |
| B5 | 32 |
| F1 | n/a |
| F2 | n/a |
| M1 | n/a |
| N1 | n/a |
| N2 | n/a |
| N3 | 27 |
| N4 | 30 |
| Q1 | n/a |
| Q2 | n/a |
| Q3 | n/a |
| Q4 | n/a |
| Q5 | n/a |
| Q6 | n/a |
| R1 | n/a |
| R2 | −20 |
| R3 | 20 |

¹n/a means datum not available

The MIA Rat data reported above in Table 1 establishes that the invention compounds are effective at preventing or treating joint cartilage damage and treating osteoarthritis.

Results of MIA studies with oral dosing may be shown in a table in columns labelled “IJFL (%+/−SEM)”, wherein IJFL means Inhibition of Joint Function Limitation, % means percent, +/− means plus or minus, and SEM means standard error measure; “SDCES”, wherein SDCES means Significant Decrease In Cartilage Erosion Severity, and “SIJWHLE”, wherein SIJWHLE means Significant Increase in Joints Without Hind Limb Erosion.

The proportion of subjects without hind limb erosions may be analyzed via an Exact Sequential Cochran-Armitage Trend test (SAS® Institute, 1999). The Cochran-Armitage Trend test is employed when one wishes to determine whether the proportion of positive or “Yes” responders increases or decreases with increasing levels of treatment. For the particular study, it is typically found that the number of animals without joint erosions increases with increasing dose.

The ridit analysis may be used to determine differences in overall erosion severity. This parameter takes into account both the erosion grade (0=no erosion, I=erosion extending into the superficial or middle layers, or II=deep layer erosion), and area (small, medium and large, quantified by dividing the area of the largest erosion in each score into thirds) simultaneously. The analysis recognizes that each unit of severity is different, but does not assume a mathematical relationship between units.

### Biological Method 3

Induction of Experimental Osteoarthritis in Rabbit (“EOA in Rabbit”):

Normal rabbits are anaesthetized and anteromedial incisions of the right knees performed. The anterior cruciate ligaments are visualized and sectioned. The wounds are closed and the animals are housed in individual cages, exercised, and fed ad libitum. Rabbits are given either vehicle (water) or an invention compound (10 rabbits per group). Each group was dosed three times per day with the invention compound group receiving 30-mg/kg/dose or 10-mg/kg/dose. The rabbits are euthanized 8 weeks after surgery and the proximal end of the tibia and the distal end of the femur are removed from each animal.

Macroscopic Grading

The cartilage changes on the femoral condyles and tibial plateaus are graded separately under a dissecting microscope (Stereozoom, Bausch & Lomb, Rochester, N.Y.). The depth of erosion is graded on a scale of 0 to 4 as follows: grade 0=normal surface; Grade 1=minimal fibrillation or a slight yellowish discoloration of the surface; Grade 2=erosion extending into superficial or middle layers only; Grade 3=erosion extending into deep layers; Grade 4=erosion extending to subchondral bone. The surface area changes are measured and expressed in mm². Representative specimens may also be used for histologic grading (see below).

Histologic Grading

Histologic evaluation is performed on sagittal sections of cartilage from the lesional areas of the femoral condyle and tibial plateau. Serial sections (5 um) are prepared and stained with safranin-O. The severity of OA lesions is graded on a scale of 0-14 by two independent observers using the histologic-histochemical scale of Mankin et al. This scale evaluates the severity of OA lesions based on the loss of safranin-O staining (scale 0-4), cellular changes (scale 0-3), invasion of tidemark by blood vessels (scale 0-1) and structural changes (scale 0-6). On this latter scale, 0 indicates normal cartilage structure and 6 indicates erosion of the cartilage down to the subchondral bone. The scoring system is based on the most severe histologic changes in the multiple sections.

Representative specimens of synovial membrane from the medial and lateral knee compartments are dissected from underlying tissues. The specimens are fixed, embedded, and sectioned (5 um) as above, and stained with hematoxylin-eosin. For each compartment, two synovial membrane specimens are examined for scoring purposes and the highest score from each compartment is retained. The average score is calculated and considered as a unit for the whole knee. The severity of synovitis is graded on a scale of 0 to 10 by two independent observers, adding the scores of 3 histologic criteria: synovial lining cell hyperplasia (scale 0-2); villous hyperplasia (scale 0-3); and degree of cellular infiltration by mononuclear and polymorphonuclear cells (scale 0-5): 0 indicates normal structure.

Statistical Analysis

Mean values and SEM is calculated and statistical analysis was done using the Mann-Whitney U-test.

The results of these studies would be expected to show that an invention compound, or pharmaceutically acceptable salt thereof, would reduce the size of the lesion on the tibial plateaus, and perhaps the damage in the tibia or on the femoral condyles. In conclusion, these results would show that an invention compound, or pharmaceutically acceptable salt thereof, would have significant inhibition effects on the damage to cartilage.

The foregoing studies would establish that an invention compound, or pharmaceutically acceptable salt thereof, is effective for the inhibition of joint cartilage damage and treatment of osteoarthritis in human, and other mammalian disorders. Such a treatment offers a distinct advantage over existing treatments that only modify joint pain and other secondary symptoms. The effectiveness of an invention compound in this model would indicate that the invention compounds will have clinically useful effects in preventing and/or treating joint cartilage damage.

The invention compounds may be tested for binding to an alpha-2-delta receptor, particularly an alpha-2-delta receptor 1 (“A2DR1”) and an alpha-2-delta receptor 2 (“A2DR2”), according to any one of Biological Methods 4 to 6 described below.

### Biological Method 4

[³H]Gabapentin A2DR1 and A2DR2 Binding Assays (“A2DR1” and “A2DR2”, Respectively)

Step (1): Preparation of A2DR1 or A2DR2 Protein

HEK 293 recombinant cells expressing A2DR1 or A2DR2 protein are harvested and washed in phosphate buffered saline (“PBS”). The cells are centrifuged and resuspended in tris(hydroxymethyl)aminomethane-ethylenediaminetetraacetic acid (“Tris-EDTA” or “TE”) buffer containing Roche Complete Protease Inhibitor Cocktail. The cells are homogenized, centrifuged at 3000×g, and the supernatant centrifuged again at 50,000×g. The resulting pellet is resuspended and homogenized in TE. Following determination of A2DR1 or A2DR2 protein concentration, aliquots are stored at −70° C. until the day of testing (membrane preparation may be used for at least 6 months).

Step (2): A [³H]Gabapentin A2DR1 and A2DR2 Binding Assay

In one version, the binding assay is set up in a 96-well format using deep-well polypropylene plates. In a total volume of 500 μL, the following additions are made: 250 μL buffer [10 mM N-2-hydroxyethylpiperazine-N′-2-ethanesulfonic acid (“HEPES”), pH 7.4], 25 μL [3H]gabapentin (10 nM final concentration), 200 μL thawed tissue membrane preparation from Step (1) (˜40 μg protein), and 25 μL of test compound at 7 test dilutions (for example, 0.001, 0.01, 0.03, 0.1, 0.3, 1.0, and 10 μM concentrations). Non-specific binding for each plate is determined by the addition of 10 μM of pregabalin instead of test compound in a few of the 96 wells. Following incubation for 45 minutes at 21° C., the contents of the wells are filtered under vacuum onto glass filter/B (“GF/B”) filter mats and then washed 4×1 mL with chilled 50 mM tris(hydroxymethyl)aminomethane (“Tris”) HCl, pH 6.9. The mats are placed into plastic pouches, scintillation cocktail is added, the pouches are sealed, and radioactivity for each sample is counted.

Step (3): Determination of IC₅₀ Values

Specific binding values for each concentration are transformed and then analyzed by a 4-parameter nonlinear regression equation provided by Prism graphics software by Graph Pad to determine IC₅₀ values by conventional means.

### Biological Method 5

[³H]Gabapentin Scintillant Proximity A2DR1 and A2DR2 Binding Assays (“A2DR1 SPA” and “A2DR2 SPA”, Respectively)

Step (1): Preparation of A2DR1 or A2DR2 Protein

Recombinant HEK 293 cells expressing pig A2DR1 and A2DR2 subunits were grown under normal cell culture conditions (RPMI-1640 media with 10% FBS, 200 μg G418, and 1% penicillin/streptomycin It 37° C. with 5% CO₂) until reaching confluency in T-75 flasks, at which time they were harvested. The harvested cells were suspended in ice-cold 5 mM Tris/5 mM ethylenediaminetetraacetic acid (“EDTA”) buffer, pH 7.4 (“TE buffer”) containing phenylmethylsulfonyl fluoride (“PMSF”) (0.1 mM) and Roche Complete Protease Inhibitor Cocktail, and allowed to sit on ice for 30 minutes. The cells were broken by sonication using 20 bursts, 40-50 cycles, and then centrifuged at 3000×g for 10 minutes. The resulting supernatant was transferred to a new tube and centrifuged at 50,000×g for 30 minutes. The resulting pellet was resuspended in 10 mM HEPES buffer, pH 7.4, homogenized, and stored at −80° C. The A2DR1 or A2DR2 membrane protein concentration was determined by the Pierce BCA method using bovine serum albumin (“BSA”) as the standard.

Step (2): Scintillant Proximity Assay (SPA).

The [³H]gabapentin SPA binding assay was performed in Costar 3632 96-well, clear bottom assay plates using Wheatgerm agglutinin beads (Amersham Pharmacia Biotech). Pig A2DR1 or A2DR2 membranes (10-20 μg protein per well) prepared above in Step (1) and SPA beads (0.5 mg per well) were mixed with 30 nM [³H]gabapentin (52 Ci/mmol; Amersham Pharmacia Biotech) in 10 mM HEPES/10 mM MgSO₄ assay buffer, pH 7.4 using KOH. The final well volume was 200 μL and non-specific binding was determined in the presence of 10 μM unlabeled pregabalin. The final mixture containing A2DR1 or A2DR2 membrane protein incubated with SPA beads, test compounds, and [³H]gabapentin was incubated at room temperature for 15-24 hours, and the plates were then counted on a Wallace Trilux 1450 Microbeta scintillation counter. Step (3): Determination of IC₅₀ values.

Curve fitting and IC₅₀ values were calculated using a four-parameter, non-linear regression equation from GraphPad Prism 3.0 software, while Kᵢ values were determined using the equation of Chang and Prussoff.

Alternatively in Biological Methods 4 or 5, test compound may be assayed at a single concentration, for example 10 μM, to preliminarily determine the presence or absence of a predetermined threshold level of binding activity.

### Biological Method 6

[³H]Gabapentin A2DR Binding Assay (“GABAPAA” or “GABAP”)

The GABAPAA assay, also known as the GABAP assay, is carried out according to the method of Biological Method 4 except that the source of alpha-2-delta receptors is membrane from pig cortex.

It should be appreciated that the A2DR1 and A2DR2 assays of Biological Method 4 may be carried out using test compounds synthesized by conventional combinatorial chemistry methods. Such assays are termed “A2DR1CCP” and “A2DR2CCP”, respectively.

It should be appreciated that the assays of Biological Methods 4 and 6 are conventional filter receptor binding assays and the assay of Biological Method 5 is a conventional SPA receptor binding assay.

Displacement of [3H]-gabapentin from an alpha-2-delta receptor-1 or an alpha-2-delta receptor-2 by certain invention compounds is evidenced below in Table 2. In Table 2, the displacement was determined according to one of the above Biological Methods 4 or 6, and reported as an IC₅₀ in micromolar concentration of invention compound in the columns labeled “A2DR1 IC₅₀ (μM),” “A2DR2 IC₅₀ (EM),” and “GABAPAA IC₅₀ (μM),” respectively, for a reference compound, racemic octahydroisoindole-2-carboxylic acid (“OHIII2CA”) and certain invention compounds, which are referenced by Compound Example number in the column labeled “Compound Example No.”.

TABLE 2
Displacement of [3H]-Gabapentin from alpha-2-delta receptors 1 or 2

| Compound | A2DR1 | A2DR2 | |
|---|---|---|---|
| Example | IC₅₀ | IC₅₀ | GABAPAA |
| No. | (μM) | (μM) | IC₅₀ (μM) |
| OHII2CA | n/a¹ | n/a | 0.03 |
| B1 | n/a | n/a | n/a |
| B2 | ≧10 | ≧10 | ≧10 |
| B3 | n/a | n/a | n/a |
| B4 | n/a | n/a | n/a |
| B5 | ≧10 | ≧10 | ≧10 |
| F1 | n/a | n/a | n/a |
| F2 | n/a | n/a | n/a |
| M1 | ≧10 | n/a | n/a |
| N1 | n/a | n/a | n/a |
| N2 | n/a | n/a | n/a |
| N3 | n/a | n/a | n/a |
| N4 | n/a | n/a | n/a |
| Q1 | n/a | n/a | n/a |
| Q2 | n/a | n/a | n/a |
| Q3 | n/a | n/a | n/a |
| Q4 | n/a | n/a | n/a |
| Q5 | n/a | n/a | n/a |
| Q6 | n/a | n/a | n/a |
| R1 | n/a | n/a | n/a |
| R2 | n/a | n/a | n/a |
| R3 | n/a | n/a | n/a |

n/a means datum not available.

The foregoing studies establish that the invention compounds, or pharmaceutically acceptable salt thereof, do not displace gabapentin from an alpha-2-delta receptor.

### Biological Method 7

Leucine Transport System Binding Assay (“LTSBA”)

The sodium-containing buffer for transport assay was phosphate buffered saline (“PBS”) consisting of 137 mM NaCl, 2.7 mM KCl, 10.6 mM Na2HPO4, and 1.5 mM KH2PO4. The sodium-free buffer had equimolar amounts of choline chloride and choline phosphate in place of NaCl and Na2BPO4, respectively. This buffer is referred to as PBC. Prior to use, both PBS and PBC buffers (pH 7.4) were supplemented with 5.6 mM D-glucose, 0.49 mM MgCl2, and 0.9 mM CaCl₂ (GMC). For cultured monolayer cells (in general, CHO K1 cells), the cluster tray transport assay was used as described previously (Su et al, J. Neurochem., 1995; 64:2125-2131, and references therein).

An appropriate amount of choline chloride was added to each reaction mixture to keep all solutions at equal osmolarity. The initial transport rates were determined by measuring uptake of tracer [3H] leucine (0.5 μCi/ml) at 37° C. for 30 seconds in the presence or absence of inhibitors at 10 concentrations from 0.3 μM to 10 mM. Secondary analysis of the initial velocity kinetic curves were determined by non linear regression analysis, using the equation: log V=log {[Vmax.S/(Km+S)]+P.[S]} where V, S, Vmax, and Km have their conventional meanings, and P represents the first order rate constant describing the nonsaturable uptake.

An IC₅₀ was determined as percentage inhibition of saturable leucine uptake.

All the transport rates were referred to as saturable uptake rates. They were calculated by subtracting the corresponding substrate uptake rates in the presence of 10 mM excess unlabeled leucine from the total uptake rates. For efflux assays, the cells were pre-incubated with 50 μM corresponding substrates for 10 minutes, then subjected to warm wash with PBC. GMC buffer twice and incubation in the same buffer in the presence or absence of substrates. The efflux was calculated as percentage of the intracellular labeled substrate at zero time. The assay protocol for 96-well plates:

Day 1: Prepare Cho-K1 cells using 96-well plates. Trypsinize cells and dilute cells to a concentration of 3×105 cells/mL and then add 100 mL of this cell suspension to each well. Culture media for Cho cells: Minimum essential medium alpha medium (Gibco #32571-036), 5% FBS—heat inactive (Gibco# 10082-139), 1% Penicillin/streptomycin (Gibco #15140-122). Use Trypsin-EDTA (Gib #25300-054) for cell passage.

Day 2: Run the assay as follows.

Step (1): Make stock: 32 μL 3H-L-Leu (1 μCi/μL) in 1L. 4 mL H2O, 1.6 mL 10×PBC, 1.6 mL 10×GMC (makes enough stock for one 96-well plate)

Step (2): Put the cold washing buffer PBS (Gibco #10010-023) on ice.

Step (3): Incubate cells with 1×GMC-PBC 2×20 minutes at 37° C. (175 μL/well).

Step (4): Prepare 96-well plates for the assay as follows: 14 μL compounds (100 mM, 30 mM, 10 mM, 3 mM, 1 mM, 300 μM, 100 μM, 30 μM, 10 μM, 3 μM)/well. Use 100 mM choline chloride and 100 mM “cold” L-leucine as controls. See Figures below. Then add 125 μL of “hot” stock/well. When calculating IC₅₀'s in Sigmaplot use Weber Hill formula, f=a/(1+abs(x/xo)\hat{ }b, and use concentrations of: (in mM) 8300, 2490, 830, 249, 83, 24.9, 8.3, 2.49, 0.83, 0.249. This is done because of dilution factors of 1/10 here and 100/120 (−17%) from residual 20 mL after aspiration of GMC-PBC left in wells when 100 mL of drug solution is added.

Step (5): Put cell culture plate and drug plate in Beckman Multimek robot (see set plate locations below). The assay automatically goes as follows: Start the reaction by adding 100 mL of the reaction media from plates prepared above. Incubate for 120 seconds at room temperature. Wash 3× with cold PBS.

Step (6): Shake out remaining PBS from plate and add 200 mL of scintillation cocktail to each well with the Brandel 96-well auto addition machine.

Step (7): Count plate using the Wallac Beta plate reader (protocol Mark 3H).

Multimek Plate Arrangement:

| | |
|---|---|
| Tip Holder | Tip wash |
| station | |
| Drug Plate | Waste |
| reservoir | |

Na⁺-Free Buffers:
10×GMC (per Liter):
Glucose—10 g
MgCl₂—1 g
CaCl₂ (anhydrous)—1 g
filter
   10×PBC (per Liter):
Choline Chloride—194 g
KCl—2 g
Choline₂HPO₄—430 ml (see how to make below)
KH₂PO₄—2 g
PH to 7.4 and filter
   To Make Choline₂HPO₄ (per Liter):
H₃PO₄ (85%, 14.7M)—17 ml
Chol₂HCO₃ (80%, 4.84M)—103 ml

Boil and stir for ˜2 hrs to remove CO₂ then adjust to pH 8.2 with HCl.

Plate Layout:

| | 1 | 2 | 3 | 4 | 5 | 6 |
|---|---|---|---|---|---|---|
| | (mM) | (mM) | (mM) | (mM) | (mM) | (μM) |
| A | 100 | 30 | 10 | 3 | 1 | 300 |
| B | 100 | 30 | 10 | 3 | 1 | 300 |
| C | 100 | 30 | 10 | 3 | 1 | 300 |
| D | 100 | 30 | 10 | 3 | 1 | 300 |
| E | 100 | 30 | 10 | 3 | 1 | 300 |
| F | 100 | 30 | 10 | 3 | 1 | 300 |
| G | 100 | 30 | 10 | 3 | 1 | 300 |
| H | 100 | 30 | 10 | 3 | 1 | 300 |

Plate layout continued:

| | 7 | 8 | 9 | 10 | | |
|---|---|---|---|---|---|---|
| | (μM) | (μM) | (μM) | (μM) | 11 | 12 |
| A | 100 | 30 | 10 | 3 | ChoCl | Leu |
| B | 100 | 30 | 10 | 3 | ChoCl | Leu |
| C | 100 | 30 | 10 | 3 | ChoCl | Leu |
| D | 100 | 30 | 10 | 3 | ChoCl | Leu |
| E | 100 | 30 | 10 | 3 | ChoCl | Leu |
| F | 100 | 30 | 10 | 3 | ChoCl | Leu |
| G | 100 | 30 | 10 | 3 | ChoCl | Leu |
| H | 100 | 30 | 10 | 3 | ChoCl | Leu |

Compound Preparation (Serial):

| 50 μL × 100 mM 6.7 μL 100 mM in | | | 6.7-60 | 6.7-60 | 6.7-60 |
|---|---|---|---|---|---|
| 60 μL ChoCl | | | | | |
| 21 μL 100 mM in 49 μL ChoCl | | 6.7-60 | 6.7-60 | 6.7-60 | 6.7-60 |
| | | ↓ | | | |
| 100 mM | 10 mM | | 1 mM | 100 uM | 10 uM |
| 30 mM | 3 mM | | 300 μM | 30 μM | 3 μM |

Leucine transport system binding data for the invention compounds and reference compound octahydroisoindole-2-carboxylic acid hydrochloride (“OHII2CA HCl”) expressed as an IC₅₀ in micromolar are shown below in Table 3 in the column labelled “LTS IC₅₀ (μM).”

TABLE 3
Leucine transport system binding

| | LTS |
|---|---|
| Compound | IC₅₀ |
| Example No. | (μM) |
| OHII2CA HCl | 8300 |
| B1 | 8300 |
| B2 | 8300 |
| B3 | 1618 |
| B4 | 8300 |
| B5 | 8300 |
| F1 | n/a¹ |
| F2 | 8300 |
| M1 | 8300 |
| N1 | 8300 |
| N2 | n/a |
| N3 | 8300 |
| N4 | 8300 |
| Q1 | n/a |
| Q2 | n/a |
| Q3 | n/a |
| Q4 | n/a |
| Q5 | n/a |
| Q6 | n/a |
| R1 | 8300 |
| R2 | n/a |
| R3 | 990 |

¹n/a means datum not available

As the data evidences, both the invention compounds and the reference OHII2CA HCl weakly bind or do not bind to the leucine transport system. Accordingly, both the invention compounds are expected to exhibit low to no penetration of a blood-brain barrier.

### Biological Method 8

Drug Plasma Half-Life Assay (“DPH-LA”)

Intravenous Infusion Protocol:

Three fasted male Sprague-Dawley rats are dosed by intravenous infusion via cannula over 5 minutes of 3 mg/kg of invention compound in 1.0 mL saline solution, and the cannulas were each rinsed with 1.0 mL saline solution immediately following infusion. Animals are fed at 4 hours post dose. Blood samples are collected by conventional means in tubes containing ethylenediaminetetraacetic acid (“EDTA”) at Times 0 (predose), 0.083 (end of infusion), 0.25, 0.5, 1, 2, 4, 6, 8, 12, and 24 hours post dose. Plasma is separated from the blood and stored frozen until analyzed by conventional high performance liquid chromatography, whereby 5.0 ng/mL was the lower limit of quantitation.

Peroral Protocol:

Three fasted male Wistar rats are dosed by oral gavage with optionally 5 mg/kg, 30 mg/kg, or 300 mg/kg of invention compound. Animals are fed at 4 hours post dose. Blood samples are collected by conventional means in tubes containing EDTA at Times 0 (predose), 0.5, 1, 2, 4, 6, 8, 12, and 24 hours post dose. Plasma is separated from the blood and stored frozen until analyzed.

Plasma Half-Life Analysis:

Mean plasma half-life was determined by thawing and assaying the plasma samples using high performance liquid chromatography by conventional means. A concentration of test compound of 5.0 ng/mL was the lower limit of quantitation.

Plasma half-life was calculated as the time in hours by conventional means. Drug quantitation is done by comparison to standard samples containing known amounts of test compound.

Bioavailability:

Mean bioavailability for test compound was determined by calculating area under the concentration-time curve (“AUC”) Extrap using conventional methods.

Administration according to the invention method of an invention compound to a mammal to treat the diseases listed above is preferably, although not necessarily, accomplished by administering the compound, or a salt thereof, in a pharmaceutical dosage form.

The invention compounds can be prepared and administered according to the invention method in a wide variety of oral and parenteral pharmaceutical dosage forms. Thus, the invention compounds can be administered by injection, that is, intravenously, intramuscularly, intracutaneously, subcutaneously, intraduodenally, or intraperitoneally. Also, the invention compounds can be administered by inhalation, for example, intranasally. Additionally, the invention compounds can be administered transdermally. It will be obvious to those skilled in the art that the following dosage forms may comprise as the active components either an invention compounds. The active compounds generally are present in a concentration of about 5% to about 95% by weight of the formulation.

For preparing pharmaceutical compositions from the invention compounds (i.e., the active components) pharmaceutically acceptable carriers can be either solid or liquid. Solid form preparations are preferred. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid which is in a mixture with the finely divided active component. Powders suitable for intravenous administration or administration by injection may be lyophilized.

In tablets, the active component is mixed with the carrier having the necessary binding properties in suitable proportions and compacted in the shape and size desired.

The powders and tablets preferably contain from about 5% to about 70%, total, of the active component. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active component with encapsulating material as a carrier providing a capsule in which the active component, with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogenous mixture is then poured into convenient sized molds, allowed to cool, and thereby to solidify.

Liquid form preparations include solutions, suspensions, and emulsions, for example, water or water propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizing, and thickening agents as desired.

Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Pharmaceutically acceptable carriers are determined in part by the particular composition being administered, as well as by the particular method used to administer the composition. Accordingly, there is a wide variety of suitable formulations of pharmaceutical compositions of the present invention (see, for example, Remington: The Science and Practice of Pharmacy, 20th ed., Gennaro et al. Eds., Lippincott Williams and Wilkins, 2000).

A compound of the present invention, alone or in combination with other suitable component(s), can be made into aerosol formulations (i.e., they can be “nebulized”) to be administered via inhalation. Aerosol formulations can be placed into pressurized acceptable propellants, such as dichlorodifluoromethane, propane nitrogen, and the like.

Formulations suitable for parenteral administration, such as, for example, by intravenous, intramuscular, intradermal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. The formulations of compounds can be presented in unit-dose or multi-dose sealed containers, such as ampules and vials. Injection solutions and suspensions can be prepared from sterile powders, granules, and tablets of the kind previously described.

The pharmaceutical preparation is preferably in unit dosage form. In such form, the preparation is subdivided into unit doses containing an appropriate quantity of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.01 to 1000 mg, preferably 1 to 500 mg according to the particular application and the potency of the active components. The composition can, if desired, also contain other compatible therapeutic agents.

In therapeutic use as agents to treat the above-listed diseases, the invention compounds or a combination of the same with valdecoxib, are administered at a dose that is effective for treating at least one symptom of the disease or disorder being treated. The initial dosage of about 1 mg/kg to about 100 mg/kg daily of the active component will be effective. A daily dose range of about 25 mg/kg to about 75 mg/kg of the active component is preferred. The dosages, however, may be varied depending upon the requirements of the patient, the severity of the condition being treated, and the invention compound or combination being employed. Determination of the proper dosage for a particular situation is within the skill of the art as described above. Typical dosages will be from about 0.1 mg/kg to about 500 mg/kg, and ideally about 25 mg/kg to about 250 mg/kg, such that it will be an amount that is effective to treat the particular disease or disorder being treated.

A preferred composition for dogs comprises an ingestible liquid peroral dosage form selected from the group consisting of a solution, suspension, emulsion, inverse emulsion, elixir, extract, tincture and concentrate, optionally to be added to the drinking water of the dog being treated. Any of these liquid dosage forms, when formulated in accordance with methods well known in the art, can either be administered directly to the dog being treated, or may be added to the drinking water of the dog being treated. The concentrate liquid form, on the other hand, is formulated to be added first to a given amount of water, from which an aliquot amount may be withdrawn for administration directly to the dog or addition to the drinking water of the dog.

A preferred composition provides delayed-, sustained- and/or controlled-release of the invention compound. Such preferred compositions include all such dosage forms which produce ≧40% inhibition of cartilage degradation, and result in a plasma concentration of the active component of at least 3 fold the active component's ED₄₀ for at least 2 hours; preferably for at least 4 hours; preferably for at least 8 hours; more preferably for at least 12 hours; more preferably still for at least 16 hours; even more preferably still for at least 20 hours; and most preferably for at least 24 hours. Preferably, there is included within the above-described dosage forms those which produce >40% inhibition of cartilage degradation, and result in a plasma concentration of the active component of at least 5 fold the active component's ED₄₀ for at least 2 hours, preferably for at least 2 hours, preferably for at least 8 hours, more preferably for at least 12 hours, still more preferably for at least 20 hours and most preferably for at least 24 hours. More preferably, there is included the above-described dosage forms which produce ≧50% inhibition of cartilage degradation, and result in a plasma concentration of the active component of at least 5 fold the active component's ED₄₀ for at least 2 hours, preferably for at least 4 hours, preferably for at least 8 hours, more preferably for at least 12 hours, still more preferably for at least 20 hours and most preferably for at least 24 hours.

The above formulation embodiments illustrate the invention pharmaceutical compositions containing a joint cartilage damage treating effective amount or an anti-osteoarthritic effective amount of an invention compound, and a pharmaceutically acceptable carrier, diluent, or excipient. The formulation embodiments are representative only, and are not to be construed as limiting the invention in any respect.

While it may be desirable to formulate an invention compound and another drug together in one capsule, tablet, ampoule, solution, and the like, for simultaneous administration, it is not necessary for the purposes of practicing the invention methods with respect to combinations.

Still further, it should be appreciated that the invention methods comprising administering an invention combination to a mammal to treat diseases or disorders listed above may be used to treat different diseases simultaneously. For example, administration of valdecoxib in accordance with the invention combination may be carried out as described above to treat joint inflammation, arthritic joint pain, pain associated with menstrual cramping, and migraines, while an invention compound may be administered to treat OA or inhibit joint cartilage damage.

As shown above, the invention method offers a distinct advantage over existing treatments for diseases such as OA that comprise joint cartilage damage, wherein the existing treatments modify joint pain or secondary symptoms, but do not show a disease modifying effect.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. It is intended, therefore, that the invention be defined by the scope of the claims that follow and that such claims be interpreted as broadly as is reasonable.

All of the references cited above are hereby incorporated by reference herein in their entireties and for all purposes.

Having described the invention, various embodiments of the invention are hereupon claimed.

1. A compound of Formula I 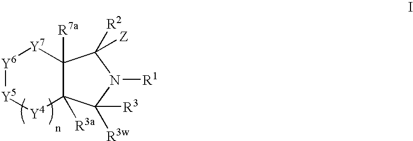
or a pharmaceutically acceptable salt thereof,
wherein:
Z is selected from COOH, C(O)N(H)R⁹, and Z¹;
Z¹ is selected from: 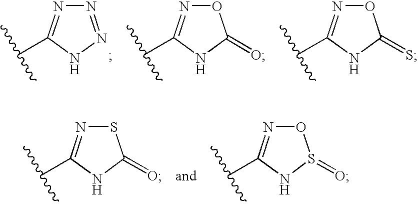
Each Y⁴, Y⁵, Y⁶, and Y⁷ is C(R¹⁰)R^{10w}; or
One of Y⁴, Y⁵, Y⁶, and Y⁷ is selected from O, S, S(O), S(O)₂, and NR⁵, and the other three of Y⁴, Y⁵ Y⁶, and Y⁷ are each C(R¹⁰)R^{10w}; or
Two nonadjacent Y⁴, Y⁵, Y⁶ and Y⁷ are independently selected from O, S, S(O), S(O)₂, and NR⁵, and the other two of Y⁴, Y⁵, Y⁶, and Y⁷ are each C(R¹⁰)R^{10w};
Each R², R³, R^{3w}, R^{3a}, R^{7a}, R¹⁰, and R^{10w} is independently selected from: H, HO, H₂N, H₂NS(O)₂-(G)ₘ, HS, Halo, CN, CF₃, FC(H)₂O, F₂C(H)O, CF₃O, and
a group, which may be unsubstituted or substituted, independently selected from:
C₁-C₆ alkyl-(G)ₘ-, C₂-C₆ alkenyl-(G)ₘ-, C₂-C₆ alkynyl-(G)ₘ-, 2- to 6-membered heteroalkyl-(G)ₘ-, 2- to 6-membered heteroalkenyl-(G)ₘ-, C₃-C₇ cycloalkyl-(G)ₘ-, C₃-C₇ cycloalkenyl-(G)ₘ-, C₇-C₁₀ bicycloalkyl-(G)ₘ-, 3- to 7-membered heterocycloalkyl-(G)ₘ-, 7- to 10-membered heterobicycloalkyl-(G)ₘ-, Phenyl-(G)ₘ-, Naphthyl-(G)ₘ-, 5- and 6-membered heteroaryl-(G)ₘ-, 8- to 10-membered heterobiaryl-(G)ₘ-, and
any of the above R², R³, R^{3w}, R^{3a}, R^{7a}, R¹⁰, and R^{10w} groups each independently substituted on carbon or nitrogen atoms with from 1 to 6 substituents R^{X};
wherein R³ and R^{3w}, and any geminal pair of R¹⁰ and R^{10w}, and any two R^{X} substituents geminally substituted on a carbon atom in substituted R², R³, R^{3w}, R^{3a}, R^{7a}, R¹⁰ and R^{10w} groups further may independently be taken together with a carbon atom to which they are both bonded to form the group C(═O);
R¹ is HO or a group that may be unsubstituted or substituted, independently selected from:
C₁-C₆ alkyl-(T)ₘ-, C₂-C₆ alkenyl-(T)ₘ-, C₂-C₆ alkynyl-(T)ₘ-, 2- to 6-membered heteroalkyl-(T)ₘ-, 2- to 6-membered heteroalkenyl-(T)ₘ—, C₃-C₇ cycloalkyl-(T)ₘ-, C₃-C₇ cycloalkenyl-(T)ₘ-, C₇-C₁₀ bicycloalkyl-(T)ₘ-, 3- to 7-membered heterocycloalkyl-(T)ₘ-, 7-to 10-membered heterobicycloalkyl-(T)ₘ-, Phenyl-(T)ₘ-, Naphthyl-(T)ₘ-, 5- and 6-membered heteroaryl-(T)ₘ-, 8- to 10-membered heterobiaryl-(T)ₘ-, and
any of the above R¹ groups independently substituted on a carbon or nitrogen atom, with from 1 to 6 substituents R^{X};
R¹ may further be H when: (i) at least one of R², R³, R^{3w}, R^{3a}, R^{7a}, R¹⁰, and R^{10w} is not H, or (ii) Z is C(O)N(H)R⁹ wherein R⁹ is as defined above wherein m is 1 and L is S(O)₂, or (iv) Z is Z¹;
Each R⁵ and R⁹ is independently H, HO, or a group, which may be unsubstituted or substituted, independently selected from: C₁-C₆ alkyl-(L)ₘ-, C₂-C₆ alkenyl-(L)ₘ-, C₂-C₆ alkynyl-(L)ₘ-, 2- to 6-membered heteroalkyl-(L)ₘ-, 2- to 6-membered heteroalkenyl-(L)ₘ-, C₃-C₇ cycloalkyl-(L)ₘ-, C₃-C₇ cycloalkenyl-(L)ₘ, C₇-C₁₀ bicycloalkyl-(L)ₘ-, 3- to 7-membered heterocycloalkyl-(L)ₘ-, 7- to 10-membered heterobicycloalkyl-(L)ₘ-, Phenyl-(L)ₘ-, Naphthyl-(L)ₘ-, 5- and 6-membered heteroaryl-(L)ₘ-, 8- to 10-membered heterobiaryl-(L)ₘ-, and
any of the above R⁵ and R⁹ groups independently substituted, on carbon or nitrogen atoms, with from 1 to 6 substituents R^{X};
wherein any 2 groups each selected from R⁵, R¹⁰, and R^{10w} that are bonded to contiguous carbon or nitrogen atoms in Formula I may be taken together with the contiguous atoms in Formula I to which they are bonded to form C═C or C═N;
wherein any 2 groups selected from R¹, R², R³, R^{3w}, R^{3a}, R⁵, R^{7a}, R¹⁰, and R^{10w} that are bonded to contiguous carbon or nitrogen atoms in Formula I may be taken together to form (i) a diradical selected from CH₂ and CH₂CH₂CH₂, (ii) a 3-membered diradical selected from: 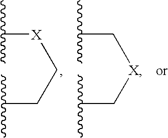
(iii) a 4-membered diradical selected from: 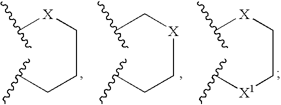
wherein any two groups R³ and R^{3w}, and R¹⁰ and R^{10w}, that are geminally bonded to a single carbon atom in Formula I may be taken together to form a 4-membered diradical as defined above or a 5-membered diradical selected from: 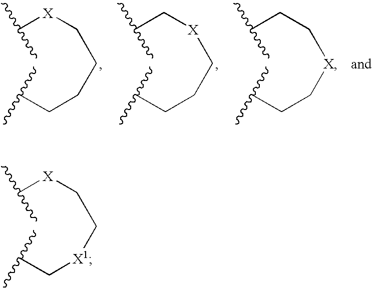
wherein any 2 groups selected from R¹, R², R³, R^{3w}, R^{3a}, R⁵, R^{7a}, R¹⁰, and R^{10w} that are bonded to noncontiguous carbon or nitrogen atoms in Formula I may be taken together to form (i) a CH₂CH₂ diradical or (ii) —O— diradical;
X is O, S, S(O), S(O)₂, or N—R;
X¹ is O or N—R;
Each G is independently selected from C(═O), S(O), S(O)₂, OC(O), N(R⁴)C(O), (C₁-C₈ alkylenyl)ₘ, (2- to 8-membered heteroalkylenyl)ₘ, and (C₁-C₈ alkylenyl)ₘ and (2- to 8-membered heteroalkylenyl)ₘ independently substituted on carbon or nitrogen atoms with from 1 to 4 substituents R^{X};
Each T is independently selected from S(O), S(O)₂, N(R⁴)C(O), (C₁-C₈ alkylenyl)ₘ, (2- to 8-membered heteroalkylenyl)ₘ, and (C₁-C₈ alkylenyl)ₘ and (2- to 8-membered heteroalkylenyl)ₘ independently substituted on carbon or nitrogen atoms with from 1 to 4 substituents R^{X};
Each L is independently selected from O, N(R⁴), S(O), S(O)₂, C(═O), C(O)O, OC(O), C(O)N(R⁴), N(R⁴)C(O), OC(O)N(R⁴), N(R⁴)C(O)o, N(R⁴)C(O)N(R^{4w}), (C₁-C₈ alkylenyl)ₘ, (2- to 8-membered heteroalkylenyl)ₘ, and (C₁-C₈ alkylenyl)ₘ and (2- to 8-membered heteroalkylenyl)ₘ independently substituted on carbon or nitrogen atoms with from 1 to 4 substituents R^{X};
Each R, R⁴, and R^{4w} is independently H or C₁-C₆ alkyl, which C₁-C₆ alkyl may be unsubstituted or substituted with from 1 to 3 substituents R^{X};
Each R^{X} is independently selected from: HO, H₂N, H₂NS(O)₂, CN, CF₃, FCH₂O, F₂C(H)O, CF₃O, O₂N, C₁-C₆ alkyl-(Q)ₘ-, 2- to 6-membered heteroalkyl-(Q)ₘ-, C₃-C₇ cycloalkyl-(Q)ₘ-, 3- to 7-membered heterocycloalkyl-(Q)ₘ-, Phenyl-(Q)ₘ, and 5-membered heteroaryl-(Q)ₘ,
wherein phenyl and 5-membered heteroaryl-(Q)ₘ each is unsubstituted or independently substituted with from 1 to 3 substituents selected from halo, HO, HOC(O), CH₃OC(O), CH₃C(O), H₂N, CF₃, CN, and C₁-C₆ alkyl;
wherein each R^{X} substituent on a carbon atom may further be independently selected from: HS, (C₁-C₆ alkyl)-S, halo, and HO₂C; and
Each Q independently is O, N(R⁶), S(O), S(O)₂, C(═O), C(O)O, OC(O), C(O)N(R⁶), N(R⁶)C(O), OC(O)N(R⁶), N(R⁶)C(O)O, or N(R⁶)C(O)N(R⁶);
Each R⁶ and R^{6w} independently is H or unsubstituted C₁-C₆ alkyl;
Each m independently is an integer of 0 or 1; and
Each n independently is an integer of from 0 to 2.

2. The compound according to claim 1 of Formula II 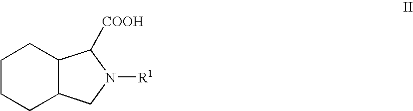
or a pharmaceutically acceptable salt thereof,
wherein R¹ is HO or a group that may be unsubstituted or substituted, independently selected from:
C₁-C₆ alkyl-(T)ₘ-, C₂-C₆ alkenyl-(T)ₘ-, C₂-C₆ alkynyl-(T)ₘ-, 2- to 6-membered heteroalkyl-(T)ₘ-, 2- to 6-membered heteroalkenyl-(T)ₘ—, C₃-C₇ cycloalkyl-(T)ₘ-, C₃-C₇ cycloalkenyl-(T)ₘ-, C₇-C₁₀ bicycloalkyl-(T)ₘ-, 3- to 7-membered heterocycloalkyl-(T)ₘ-, 7-to 10-membered heterobicycloalkyl-(T)ₘ-, Phenyl-(T)ₘ-, Naphthyl-(T)ₘ-, 5- and 6-membered heteroaryl-(T)ₘ-, 8- to 10-membered heterobiaryl-(T)ₘ-, and
any of the above R¹ groups independently substituted on a carbon or nitrogen atom, with from 1 to 6 substituents R^{X};
Each T is independently selected from S(O), S(O)₂, N(R⁴)C(O), (C₁-C₈ alkylenyl)ₘ, (2- to 8-membered heteroalkylenyl)ₘ, and (C₁-C₈ alkylenyl)ₘ and (2- to 8-membered heteroalkylenyl)ₘ independently substituted on carbon or nitrogen atoms with from 1 to 4 substituents R^{X};
Each R⁴ is independently H or C₁-C₆ alkyl, which C₁-C₆ alkyl may be unsubstituted or substituted with from 1 to 3 substituents R^{X};
Each R^{X} is independently selected from: HO, H₂N, H₂NS(O)₂, CN, CF₃, FCH₂O, F₂C(H)O, CF₃O, O₂N, C₁-C₆ alkyl-(Q)ₘ-, 2- to 6-membered heteroalkyl-(Q)ₘ-, C₃-C₇ cycloalkyl-(Q)ₘ-, 3- to 7-membered heterocycloalkyl-(Q)ₘ-, Phenyl-(Q)ₘ, and 5-membered heteroaryl-(Q)ₘ,
wherein phenyl and 5-membered heteroaryl-(Q)ₘ each is unsubstituted or independently substituted with from 1 to 3 substituents selected from halo, HO, HOC(O), CH₃OC(O), CH₃C(O), H₂N, CF₃, CN, and C₁-C₆ alkyl;
wherein each R^{X} substituent on a carbon atom may further be independently selected from: HS, (C₁-C₆ alkyl)-S, halo, and HO₂C; and
Each Q independently is O, N(R⁶), S(O), S(O)₂, C(═O), C(O)O, OC(O), C(O)N(R⁶), N(R⁶)C(O), OC(O)N(R⁶), N(R⁶)C(O)O, or N(R⁶)C(O)N(R^{6w});
Each R⁶ and R^{6w} independently is H or unsubstituted C₁-C₆ alkyl; and
Each m independently is an integer of 0 or 1.

3. The compound according to claim 2, wherein R¹ is unsubstituted or substituted C₁-C₆ alkyl-(L)ₘ.

4. The compound according to claim 1, selected from:
1,2-dimethyl-octahydro-isoindole-1-carboxylic acid hydrochloride;
1-methyl-octahydro-isoindole-1-carboxylic acid hydrochloride;
5,6-Dimethoxy-octahydro-isoindole-1-carboxylic acid;
cis-2,3,3a,4,7,7a-Hexahydro-1H-isoindole-1-carboxylic acid hydrochloride;
diastereomer 1 of 6-chloro-2,2-dimethyl-octahydro-[1,3]dioxolo[4,5-f]isoindole-5-carboxylic acid; and
diastereomer 2 of 6-chloro-2,2-dimethyl-octahydro-[1,3]dioxolo[4,5-f]isoindole-5-carboxylic acid; or
a pharmaceutically acceptable salt thereof.

5. The compound according to claim 1 selected from:
3-aza-6,6-oxabicyclo[4.3.0]nonane-2-arboxylic acid;
10-oxa-4-aza-tricyclo[5.2.1.0^{2,6}]decane-3-carboxylic acid;
4-methyl aza-tricyclo[5.2.2.0^{2,6}]undecane-3-carboxylic acid hydrochloride; and
4-aza-tricyclo[5.2.2.0^{2,6}]undecane-3-carboxylic acid hydrochloride; or
a pharmaceutically acceptable salt thereof.

6. The compound according to claim 1, selected from:
3-aza-6,6-difluorobicyclo[3.3.0]octane-2-carboxylic acid;
3-aza-6-fluorobicyclo[3.3.0]octane-2-carboxylic acid;
3-aza-6-n-butoxybicyclo[3.3.0]octane-2-carboxylic acid;
3-aza-6-hydroxybicyclo[3.3.0]octane-2-carboxylic acid hydrochloride;
3-azaoxobicyclo[3.3.0]octane-2-carboxylic acid hydrochloride;
octahydro-pyrrolo[2,1-a]isoindole-9b-carboxylic acid hydrochloride; or
a pharmaceutically acceptable salt thereof.

7. The compound according to claim 1, selected from:
7a-benzyl-octahydro-isoindole-1-carboxylic acid hydrochloride;
7a-methyl-octahydro-isoindole-1-carboxylic acid;
3,3-dimethyloctahydro-isoindole-1-carboxylic acid hydrochloride;
3-(octahydro-isoindol-1-yl)-4H-[1,2,4]oxadiazol-5-one; and
3-(2-methyl-octahydro-isoindol-1-yl)₄H-[1,2,4]oxadiazol-5-one; or
a pharmaceutically acceptable salt thereof.

8. A pharmaceutical composition, comprising a compound according to claim 1, or a pharmaceutically acceptable salt thereof, and a pharmaceutically acceptable carrier, diluent, or excipient.

9. A pharmaceutical composition, comprising a compound according to claim 2, or a pharmaceutically acceptable salt thereof, and a pharmaceutically acceptable carrier, diluent, or excipient.

10. A method of treating joint cartilage damage, osteoarthritis, rheumatoid arthritis, or joint inflammation, or alleviating joint pain, in a mammal suffering from joint cartilage damage, osteoarthritis, rheumatoid arthritis, joint inflammation, or joint pain, respectively, comprising administering to the mammal a compound according to claim 1, or a pharmaceutically acceptable salt thereof.

11. A method of treating joint cartilage damage, osteoarthritis, rheumatoid arthritis, or joint inflammation, or alleviating joint pain, in a mammal suffering from joint cartilage damage, osteoarthritis, rheumatoid arthritis, joint inflammation, or joint pain, respectively, comprising administering to the mammal a compound according to claim 2, or a pharmaceutically acceptable salt thereof.
